SEQUENCE LISTING (59 sequences)

SEQ ID NO: 1 - DNA, 589 bp, Trichoderma koningiopsis
atgaagttctccagcgtcctctcttacctcgctctcggcgtctctgccgctgttgcagctcccgctgtcgaagtcgaagtcaatatggccaatcttcccggcctcaacgcattacaagccaaatacgccaacgccatcattgcacaagccaagaaggacggtgtcggcgcacagggctgccaggcggctatcgcagctgccatggtcgaggttcgtctagccaagatacgcttatcgctattattgttctagttttactaactataaaccgtctatgtagtccagcatcatcatgtacgccaacaggggggtccccgaatctctcaagttcccccacgatcgcgttggttccgactatgatcgcatcggtctcttccagcagcctgcttccatctacaggaacatcaagtgtgacatggacgccgcttgctctgccggtcagttctttgccgaaatgaagaaatttaaggactggcaaaccatgccggttggcacgctatgccagaagattcagcgttctacttaccccgaccggtatgctaagcaggtcggcttggcgaccaatgtttgcaaggctggcggtttgtga

SEQ ID NO: 2 - protein, 153 aa, Trichoderma koningiopsis
APAVEVEVNMANLPGLNALQAKYANAIIAQAKKDGVGAQGCQAAIAAAMVESSIIMYANRGVPESLKFPHDRVGSDYDRIGLFQQPASIYRNIKCDMDAACSAGQFFAEMKKFKDWQTMPVGTLCQKIQRSTYPDRYAKQVGLATNVCKAGGL

SEQ ID NO: 3 - protein, 153 aa, Trichoderma koningiopsis; identical to SEQ ID NO: 2

SEQ ID NO: 4 - DNA, 838 bp, Thielavia terrestris
atgcagctctccctcctcgtcctctccctcgtggccgctgtgcccatggccagcgcgtacccggtcaaggccgacactctcaactgccgctccggcccgggcaccagttacaaggtcatcaagacctacaagaagggcaccgatctcaagatcacctgccagacgcccggcacctcggtcaacggcgacaacctgtgggacaagacctcggacggctgctacgtggccgattactacgtcaagaccggcacctccggctacgtcacggcccattgcgatgccggcagcggcagcggcagcagcggcggcggcaacctgccaggactcaactcggtccagtcctcgcacgcccgggccatcatcggcgaggcgaagaaggagggcgtcggccgccacggctgcgaggccggcatcgcgaccgcgcttgtcgaggtacgttgcatcctaacatcaacacttacttgccttgaccccactgtcaccgccagaaaaaaccaaaactaacacatcacctcttcccctcacacagtccaacatcctgatctacgccaacaaggcggtcccggcctcgctcaagtacccgcacgacgcggtgggctcggaccacgacagcgtcggcatcttccagcagcgcgccaagtactaccccaacatcgcggccgacatggacccggcgcgctcggccgcccagttcttcgccaagatgaagggcatcaagggctggcagagcatggccgtcggcacgctctgccagaaggtccagggctccgcgtacccggaccgctatgccaagcgggtctcggaggcgaccaagatttgccaggctggtgggttgtaa

SEQ ID NO: 5 - protein, 227 aa, Thielavia terrestris
YPVKADTLNCRSGPGTSYKVIKTYKKGTDLKITCQTPGTSVNGDNLWDKTSDGCYVADYYVKTGTSGYVTAHCDAGSGSGSSGGGNLPGLNSVQSSHARAIIGEAKKEGVGRHGCEAGIATALVESNILIYANKAVPASLKYPHDAVGSDHDSVGIFQQRAKYYPNIAADMDPARSAAQFFAKMKGIKGWQSMAVGTLCQKVQGSAYPDRYAKRVSEATKICQAGGL

SEQ ID NO: 6 - protein, 227 aa, Thielavia terrestris; identical to SEQ ID NO: 5

SEQ ID NO: 7 - DNA, 735 bp, Tilletia indica
atgagggtcgccttgtcggtctcgctcttgttgtccctcgcatccttctccgcagcggtgcagacctgtgtcaacacaaacggaatcgtcggagagtgtatttcgacatcgtcctgttccggtcagggtggttcgtccgatcctgcgaacctctgtcctggcgatgactccatccagtgttgtacgtggtcgggctcgtcgtgtaacggccagtgtatgccctacttgggttgttcgggaacgtccgatcccgcaaacctctgtcctggcggtgacgaggtgcagtgttgtgagggaggccatggagccctctccggatacaacgcactccagtcggcacacgcctatcagattggagcagccgtgaaagatgcaggcttgaaccgccaggcctgtttggcagccattacaacgggtattaccgaggcaaacctcctcaactatgcgaactccgatgtggacgcatcctataactacttgtacgatgccgtctcgtcggactacgactccgtcggtgtcttccagcagagggtgtcctactaccccgatttggacgcagacatggaccccaaacaggcagcagcagccttcctcaagaaaatggtcaacgtcgacggctgggagacaatggatgtgggagagctctgtcagaccgtccagggttcggcgtaccctactcgctattccgataacgtctcctcggcacagaacatctgttcggccattggattctaa

SEQ ID NO: 8 - protein, 225 aa, Tilletia indica
VQTCVNTNGIVGECISTSSCSGQGGSSDPANLCPGDDSIQCCTWSGSSCNGQCMPYLGCSGTSDPANLCPGGDEVQCCEGGHGALSGYNALQSAHAYQIGAAVKDAGLNRQACLAAITTGITEANLLNYANSDVDASYNYLYDAVSSDYDSVGVFQQRVSYYPDLDADMDPKQAAAAFLKKMVNVDGWETMDVGELCQTVQGSAYPTRYSDNVSSAQNICSAIGF

SEQ ID NO: 9 - protein, 225 aa, Tilletia indica; identical to SEQ ID NO: 8

SEQ ID NO: 10 - DNA, 747 bp, Acremonium alcalophilum
  CDS (1)..(744)
  sig_peptide (1)..(69)
  mat_peptide (121)..(744)
atgaagttcttcaccaccatcctcagcaccgccagccttgttgctgctctccccgccgctgttgactcgaaccataccccggccgctcctgaacttgttgcccggagtcctattcgtcgacgcattcccggattcgatatctcgggatggcagccgacgacggacttcgcaagggcgtacgcaaacggagaccgattcgtgtacatcaaggcaacagagggaacaacattcaaatcgtcggcattctccaggcagtacaccggagcaacccagaacggcttcatccgaggagcctaccacttcgcccagcctgcagcctcctcgggagcagcccaggcaaggtacttcgcatcgaacggtggcggttggtccaaggacggtatcaccctccctggtgccttggatatcgagtacaaccccaacggagcaacatgttatggtctctcgcagtcggcgatggtgaactggattgaggacttcgtgacaacctaccacggcatcacctcgaggtggcctgtgatctacaccacaaccgactggtggacgcagtgtaccggcaactccaaccgattcgcgaacaggtgtccgctctggatcgcgaggtatgcctcctccgtcggcaccctcccgaacggatggggcttctataccttctggcagtacaacgataagtacccccagggaggagattccaactggttcaacggtgatgcatcgaggctcagggcattggcgaacggcgattag

SEQ ID NO: 11 - protein, 248 aa, Acremonium alcalophilum
MKFFTTILSTASLVAALPAAVDSNHTPAAPELVARSPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 12 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 13 - DNA, 690 bp, Cladorrhinum bulbillosum
atgaagctcctccccctttccaccaccttattgcccgtggccctcctcgccaccgaggcctcggcggcagtccaaggcttcgacatctcgcactaccaatcctcagtcaacttccaggcggcctacaactcgggcgcccgcttcgtcatcatcaaggcgacagagggcacgacctacatcgaccccaagttttcgtctcactacacgggagccaccaacgccgggctaatccggggcgggtaccactttgcccatccggactcgtcgaccggcgccgcgcaggcagattttttcctcgcccacggcggcggctggtccggcgacggcatcaccctgcccgggatgctcgacctcgaatccgtctccggaaaggcgacctgcttcgggctctcggcctcgtccatggtcgcctggatcaagtcgttctctgaccggtaccacacccggaccggacggtacccgatgctgtacaccaacccgtcttggtggaccacctgcaccggaaacagcaacgcgttcgtcaacacgaacccgctcgttctggctcggtacgccagcgcgcccgggaccatccccggtggatggccgtatcagaccatctggcagaattcggactcgtatacctacggaggggattcggatatttttaacggcgcgctgagtgggttgcaaaagttggccagcggttaa

SEQ ID NO: 14 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 15 - protein, 207 aa, Cladorrhinum bulbillosum; identical to SEQ ID NO: 14

SEQ ID NO: 16 - DNA, 734 bp, Onygena equina
atgttgaaaacaattatctataccacccttgccgtcgctagcctggcgtcagcagccgttcccggtatcgacgtgtcgggctaccaaggcaacgtgaactgggcgaacgtcgccaacgctggaaagaagtttgcctacgtcaaggtatgcgtctccgtaatgagttatgaattggaactaatcaaatcaatcgggcataggccacggaacataccaactacatcaacccttacttcgcccagcagtacaatggcgcctacaaccagggcattattcgaggtgcataccactacgcccaccccaacggcgcaagcggagcttctcaggccaactacttccttgctcacggtggcggctggtctgctgatgggaaaacccttcctggtgccgtcgacctcgagtacggacccaatggcagcacttgctggggtatcagtcaatcggcgatgatcgcttggatccgtgacttctccaacacctaccgtgccaagaccggccggcctccagtcatctacaccagcacctcttggtggaagacctgcaccggtaactatggcggtttcggaaacgataatcccctttggattgctcgttattcaagcactgtcggcgaacttcctgctggctggcctttccacagcatctggcagaacaacgataacagcggtgttggaggggacggtgatatctggaacggtgacctggctggattgcaacgatttgcaaagggctaa

SEQ ID NO: 17 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 18 - protein, 207 aa, Onygena equina; identical to SEQ ID NO: 17

SEQ ID NO: 19 - DNA, 946 bp, Trichophaea saccata
atgcacgctctcacccttctcaccgcaaccctcttcggtctcgcagcggcctacccagtgaagaccgaccttcactgccgctcctctcccagcacttccgccagcatcgtccgcacctactccagtggaacggaagtccagatccagtgccagaccacgggcacttcggtccaaggatccaatgtctgggacaagacccagcacggttgctacgtcgcagactactacgtcaagaccgggcattctgggattttcaccaccaagtgcggtagcagctcgggtggaggttcctgcaagcctcccccgatcaatgctgctactgtcgcattgatcaaggagtttgaggggtaagtgacagctctgagtgaggtggtatgaggattaagactgacgaggatagattcgttcctaagcccgccccggatcctattggattgccgaccgtgggatacgggcatctttgcaagactaagggctgcaaagaagtgccttacagcttccctctcacccaggagactgccaccaagttgcttcagagcgatatcaagactttcacctcttgcgttagcaactacgtcaaggactctgttaagctcaacgataaccagtacggaggtgagttccagtgtaacagtgaatttattgatgatattctaagtaattttagctctggcgtcttgggctttcaacgtcggctgcggaaacgtccagacttcttcgctgatcaagagattgaacgctggggagaaccctaacactgtcgctgctcagggtaagatatttatcccggatttgctcttgacacatggctgaaaaagttgcagaactccccaagtggaagtacgctggtggaaaggttatgcctggcttggtccgccgccgcaatgctgaggtcgcgctcttcaagaagcccagcagcgttcaggcccaccctcccaagtgctaa

SEQ ID NO: 20 - protein, 262 aa, Trichophaea saccata
MHALTLLTATLFGLAAAYPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 21 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 22 - DNA, 810 bp, Pleurotus ostreatus
atgaagtacttgctcgcgtccctcgtgtccctcgtcttctccgcctccctcactcatgcggcagtcaacggagcatgttccgtgaacggcactcccggtgtgtgtttggcgtcggcctcgtgtactggctccggaggcacaacacacgtcggcttctgtcccaacgatcctgcggatatcaagtgttgtactaagactgcatgtggctcgggaggaaactgtcgcttcacttcctcctgttcgactggcaacatcgcgtccggcttgtgtcccggtcccaccgatttcaagtgttgtttgcctgcctccactggaggaggtgcatgtcctcctgcgatcaactcggcaacagtgtcgctcatcaaggaattcgagggattcgtcgcacgacctgcacccgatccgatcggattgcctactgtcggttacggccacttgtgtcagactacctcctgttcggaagcaggagcgttccccttgactgaagcaagggcaaccacgttgctcttgtcggattccagggtggccacttcgtgtctcaacaccgccatctcccgcaacgtgcgattgaacgcgaaccagttcggagcgttgacttcgtggactttcaacgtcggttgtggaaacatgcgatcgtcctcgttgttgtccaggctcaacgcaggcgaagcacccaacactgtcgcagcccaggaactccctaagtggaacaaggcaggaggccaggtgttggcaggcttgacccgaaggagggcagcggaggtcgtcctcttccagaccgcgtcgtccgtgatcgcccatccttgttaa

SEQ ID NO: 23 - protein, 249 aa, Pleurotus ostreatus
AVNGACSVNGTPGVCLASASCTGSGGTTHVGFCPNDPADIKCCTKTACGSGGNCRFTSSCSTGNIASGLCPGPTDFKCCLPASTGGGACPPAINSATVSLIKEFEGFVARPAPDPIGLPTVGYGHLCQTTSCSEAGAFPLTEARATTLLLSDSRVATSCLNTAISRNVRLNANQFGALTSWTFNVGCGNMRSSSLLSRLNAGEAPNTVAAQELPKWNKAGGQVLAGLTRRRAAEVVLFQTASSVIAHPC

SEQ ID NO: 24 - protein, 249 aa, Pleurotus ostreatus; identical to SEQ ID NO: 23

SEQ ID NO: 25 - DNA, 640 bp, Cladosporium sp-9768
atgaagctttccgctgttactgcgttgcttgctctcagcctcactgccctcgctgctccagcgcccaacccggaggagcctgccatatcagacctcgaggcccaagccgtcaaatctccaaagatccctggctacgaccagaaacagtctaacaatgcctgggctatcatcaagcagatcaaagctgagaagtttgggaagaatgctctgacagcttgtaaaatcgccttcgccaccgccatcgtcgagtccaacatctacatctacgccaacgagaaggtccccgagtccttaaaatatccgcacgacgaggtcggcaaggactacgatagtgttggtgtcttccagcagcgcgtcaagtactaccctctcgccgaagccatcgatccggccaagtctgcacatttgtttttcaagaagctgaggaaagtggaagactgggaaaaggcgaagacagacaagcagatcggtgccgtctgtcagaagatccaggtctctggtaagagtgcagaatctcgctcaacttgtaattcgagacatgcggctaacctgaagcttgtgctttacagcttacccagcaaagtatccgaagcaagtcgacgaggctgctaaaatctgcaaggctggcaagatcagttga

SEQ ID NO: 26 - protein, 171 aa, Cladosporium sp-9768
APAPNPEEPAISDLEAQAVKSPKIPGYDQKQSNNAWAIIKQIKAEKFGKNALTACKIAFATAIVESNIYIYANEKVPESLKYPHDEVGKDYDSVGVFQQRVKYYPLAEAIDPAKSAHLFFKKLRKVEDWEKAKTDKQIGAVCQKIQVSAYPAKYPKQVDEAAKICKAGKIS

SEQ ID NO: 27 - protein, 171 aa, Cladosporium sp-9768; identical to SEQ ID NO: 26

SEQ ID NO: 28 - protein, 251 aa, Chaetomium thermophilum var. thermophilum
AINDACSVNGTPGVCLSTTTCSNGGGTSTAGYCPNDPSNVRCCTKKCGSGGTCRFESTCSTGNTATGLCPGPSNFKCCLPASGGGGNCTPKAINKKTLDLLKEFEGWAASPYKDVAGYPTVGYGHKCSKNDCSELGYKFPMTKAQGEELLAKDVKGFEKCISDYINDTIKLNDNQYGALVSWSFNVGCGAAKDSTLISRLNKGDSPNTVAGEELPRWNKAGGKVVDGLTNRRKKEVELFKTSSTVIAHPPC

SEQ ID NO: 29 - protein, 155 aa, Acremonium alcalophilum
VGPAINSAALNLIKEFEGWRPNIYRDPVGLPTVGYGHLCRDSSCSDVPYPIPLSVANGERLLRSDLATASSVVLNANQYGALVSWAFNVGCGATSTSTLIRRLNAGESPNTVAAQELPRWNKAGGQVLPGLVRRRAAEVELHRTSTSVRALPACS

SEQ ID NO: 30 - protein, 251 aa, Coprinopsis cinerea okayama; identical to SEQ ID NO: 28

SEQ ID NO: 31 - protein, 160 aa, Rasamsonia brevistipitata
APSFLEARNAAPINAGALQLIENLEGFRANYYYINGHQTIGYGHDCTEKKDCGSIHPPLTQAQGAALLKKDLTEYEDCVCAMANAKDLNANEYGALVSFAYNSGCGGVQEWWHGAMAEKNFKGICEALPNTNTLGGELKSRREKEGHFCSLPTNDKSGCA

SEQ ID NO: 32 - protein, 208 aa, Acremonium alcalophilum; identical to SEQ ID NO: 12

SEQ ID NO: 33 - protein, 201 aa, Poronia punctate
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 34 - protein, 201 aa, Aspergillus deflectus
ALQGFDISNWQSSVDYQGAYDSGARFVMIKATEGTSYVDTVFNTHYTGATKAGLIRGGYHFARPDASSGAEQAEYFLAHGGGWSSDGITLPGMLDLEAGCYGLSASEMVSWIQDFGEAYKKETTRYPMIYTSTSWWSSCTGNNADFGDYPLVVARWASSVGTLPASWDAQTSWQYSDSYEYGGDAQYWNGDEEDLIAFATN

SEQ ID NO: 35 - protein, 231 aa, Poronia punctate
AVNEDAEGACDPPQGGENACGPNGSEAWLNTGLTDSTGWNPPFLDINNLSHISLEEYYAGVGATCAQYDDAFKSSGEKYGIDPAILAFIAMQESSCNADAGGPTPGLMQCEPTNCPNGQGSCQYPVEDNTDCGAKVLADALEAAGGNAVRALGAYNGWFTAEDWGASMNDGNGLTVDYPCGKGQEHGAPQNLDYLHETLNGWFQGKDIYGGDAYLDGTYACAGSCNNGDVC

SEQ ID NO: 36 - protein, 307 aa, Paecilomyces sp.
YPVTADSLNCRESPSTSAAIKKTYARNANINISCQTSGPAVKGNSIWDKTQDGCYVSDYYVKTGSSGYVTTKCGGGSSATHFGGIPSVGVLYTSDVKKHFCSASVIQSRSGNVILTAGHCVSGNGKGLRFAPGYHDGFTPYGTYPIEAAYVHANWNKNHDINFDFAFLTLGKGTYDGKSVNVQQVTGGNQLITNAGYEQSIQVVGYNNNEQQPVHCQINTYEAKAGQLGFNCGPFKGGTSGSPWMAGYNANTKHGNVIGNIGGWHTGGCSDNTSYSAKYGAGTQEVFNRANTGSRGDNVKGGASSGC

SEQ ID NO: 37 - protein, 227 aa, Hamigera sp
YPITSSGVNCRSGPGTNYDVVKTYDLGEDISITCQATGTDIYGDELWDLTEDGCYVTDYYVSTGTSSYVTSPCDGGDSGSSGSDGDLPGLDSTQSAHARDIIAEAQAEGLGHQGCTAGIATALVESNILIYANSDVPESLNYPYDAVGSDYDSVGIFQQRATYYPDIAADMDAAQSAAQFFAQMQNIDGWETMDIGELCQKVQVSAYPDRYAEREAEASDICTAGGL

SEQ ID NO: 38 - protein, 165 aa, Penicillium citrinum
AGCPGVNQAALNLVEGFEGFVSSPKPDPIGLPTVGYGHLCKSKGCSEVPYKFPLTKPNAEALLGSDLRTFTGCLDNYINNKVKLNDNQWGALASWAFNVGCGNVHTSSLIKRLNAGQSPNTVAAQELPQWNKGGGKVLPGLVRRRAAEVKLFQTPSSREAHPSCQ

SEQ ID NO: 39 - protein, 248 aa, Pyronema domesticum
ALNGPCSVGGTPGTCISTSTCSAGGGTSYSGYCPNDPTDIKCCTKTACGSGGNCRFTSQCASGNTVSGLCPGPADFKCCLPATGCTPPPVNTATVNLIKEFEGFVPSPSPDPIGLPTVGYGHLCQQTNCAEVPYPFPLTTTTAAALLQTDLKTYTKCISDYIVDSVRLNDNQYGALSSWAFNVGCGNAKTSTLIARLNAGQNPNTVASEELPKWNKAGGSVLPGLTRRRAAEVTLFTTASTKIAHPAC

SEQ ID NO: 40 - protein, 241 aa, Thielavia sp
YLITGDTVNCRSGPGTNYAVKKTYSKGTDVKITCQTSGTNVEGNSIWDKTSDGCYVADFYVKTGSNGYVTTKCGDSQTCVAPRSNQATVDLIAEFEGFVPNIYNDPVGLPTVGYGHLCKDKKCSDVKYSIPLSKADGKKLLADDMKRFEKCITAMVDKTLNLNQYGALVSWSFNVGCGAAETSTLVKRLNKGESPNTVISEELPKWVHAGGKVLAGLVRRRNAEIALAKKATDDKALPAKC

SEQ ID NO: 41 - protein, 249 aa, Chaetomium sp.
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 42 - protein, 253 aa, Metarhizium iadini
FPVTTDSLNCRAEPNTSSAVKKTYKKSADVKISCQTEGPSINGNSIWDKTQDGCYVADYYVKTGSSGYVTGKCGGGGGGGSNPPPSGGFCKTLNKAGTDLITRWEGFVDRPKPDPIGLPTVGYGHLCQKKNCAEVKYKFPLTKATALQLLNDDLPNYTKCLGKALNTGKVKLNDNQWAALTSWVFNVGCGAAQSSTLAKRLNNGENANTVASEELPKWKMGGGKVLPGLVKRRADEVKLFKTASSKTAFPTCQ

SEQ ID NO: 43 - protein, 166 aa, Aspergillus deflectus
ACVGPPVNQATLDLLKGFEKFVGEVYDDGYGNPTIGYGHLCADWSCSDVSFPQPLTEDTASQLLAGDIVGYQDAVTNALADPVTLNDNQYGALVSWTYNVGNGAMSSSSLVQRLNEGQDVVTVVNEELPQWNLVDGVVSDGLVRRREAELAVFNTPSDVGALPVAC

SEQ ID NO: 44 - protein, 171 aa, Sporormia fimetaria
SPVEGRACTGPNVNTATVALIKEFEGFVASPEPDPIGLPTVGYGHLCQTSGCTEVPYSFPLSEAEATSLMQSDLKSPQQTITLRTNSGVTLNANQYGALVSWAFNVGPGNVESSTLLSRLNAGENEGTVIAEELPKWNKAGGQTLPGLVRRRNAEVALAQTATSVGALPAC

SEQ ID NO: 45 - protein, 166 aa, Lecanicillium psalliotae
ACIGPAVNTATVDLVSQFEGFRGSPYIDATGHPTVGYGHLCQNRGCTDVKYPKPLSQADGKKLLAQDLGSAQQCITLATAGPVRLNANQYGALVSWAFNVGCGNVRKSSLLRRLNKGENPKAVIESELPQWNKGNGKPIAGLTRRRKAEVALANTPTSQGALPAKC

SEQ ID NO: 46 - protein, 242 aa, Trichocladium asperum
YLITGTTVNCRSGPGTSYAVKKSYTKGQDVKISCQQAGTSVEGNNIWDKTQDGCYVADKYVKTGVDGYVTSKCGVTTCTAPKVNAATVDLIAEFEGFRANIYTDATGHPTVGYGHLCTQSKCAEVKYKIPLSQADGKKLLADDIKKYEKCVTSMAKSKAKLNANQYGAVVSFTFNLGCGAAEGSQMMKRLNNGENPNTVIANEFPKWVHGNGAVLPGLVRRRNAEIALAKKATSTAALPPKC

SEQ ID NO: 47 - protein, 219 aa, Clavicipitaceae sp-70249
APTPDSNTVKGFYLPHHDPFWPNVDFKKAYSKGARFVFIRGGYDGILTNARFNKLWTDAKQQGLVRGAVHDIHPHSKHNATTQAKTFAEHRGKWTNDGYTLPGSLHATEYMDKKECFGLSATTLVDWISDFVTTYHSATNRYPFIFTTYGWWKNCTGNTDMFKDTAPLFLVHYGDKVGSIPGGWANQTFWQFGKVGAWAALEDEFNGDEAALKNLASTS

SEQ ID NO: 48 - protein, 207 aa, Thielavia terrestris
TVQGFDISHYQPNVDFAAAYNAGARFVIIKATEGTSYIDPSFSSHYTGATKAGLIRGGYHFAHPGETTGAAQADYFIAHGGGWTPDGITLPGMLDLESESNGECWGLSASAMVAWIRDFSDRYHERVGVYPMLYTNPSWWQTCTGNSNAFVNTNPLVLAHYSSSVGTIPGGWPYQTIWQNSDSYKYGGDSDIFNGSLDRLQALAKGS

SEQ ID NO: 49 - protein, 198 aa, Westerdykella
QGTNYNFAGAYSSGARFVIIKQPKGPPTRIPNFLLTILRPPDAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 50 - protein, 207 aa, Onygena equina; identical to SEQ ID NO: 17

SEQ ID NO: 51 - protein, 205 aa, Ovatospora brasiliensis
FDISNWQPAGTVDFAAAYSSGARFVIIKATEGTSYIDPSFSSHYTKATNAGFIRGGYHFAHPGETTGAAEADYFIAHGGGWSNDGITLPGMLDLESENKGQCWGLSTSAMVSWIKSFSDRYKSRTGVYPLIYTNPSWWSACTGNSNAFVNTNPLVLARYASAPGTIPGGWPYQTIWQNSDSYAYGGDSDIFNGSLDRLKALAKGS

SEQ ID NO: 52 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 53 - protein, 230 aa, Ovatospora brasiliensis
YPVKADSLNCRSGPGTSYKVVKTYKKGADIKISCQTEGPSVNGDNLWIKTQDGCYVADYYVKTGTNGYVAKKCSSGGSTGGGSGGGKGNLPGLNAKQSSHARAIVAQAKKDGVGLHGCEAGIATALVESGIKVYANKKVPASLKYPHDAVGSDHDSIGIFQQRAVYYPNIAADMDPARSAHQFFAKMKGVSGWKTMAVGKLCQKVQVSAYPDRYAKRVSEATKICKAAGI

SEQ ID NO: 54 - protein, 230 aa, Penicillium wellingtonense
YPITGDVVNCRSGPGTTYDVVKSYKLNADVSITCQAPGTDVKGDSVWDKTADGCYVADYYVKTGSSSYVTTKCGGDDDGGDNDGGSSGNLPGLTSTQSKHAKDIIAEAKSEDLGRQGCLAGIATAIVESNILIYANSGVPESLKYPHDAVGSDHDSVGIFQQRAMFYKDIAADMDAGKSAGQFFGKMKAVSGWKSMDVGTLCQKVQGSAYPSRYAEQVSKAEKICKAGGL

SEQ ID NO: 55 - protein, 304 aa, Aspergillus sp.
YPVKADVHCRSGPGTSYSIVKTYSTGTQISVSCQAAGTDVDGDQLWDKTSDGCYVSDYYVSTGSSNYVTSHCPTEYAIKTDVNCRSGPGTNYGIVKTYNQGVMVSLNCQASGTDVDGDSLWDKTSDGCYVSDYYVATGSSSYVTSACSGSSSSGGGGSSSSGNLPSLDSTQSAHARAIIGEAKSQNVGRQGCLAGIATALVESSMLMYANSNVAASLSYPHDAVGSDYDSVGLFQQRVSIYTNLAADMDAAQSAGQFFDEMKKVSGWETMNVGDLCQEVQRSAYPDRYAGEVSTAESICSAGGL

SEQ ID NO: 56 - protein, 222 aa, Chaetomium sp
YHIKGSIVNCRAGPGTSHAVKKTYAKGQNVQISCQTTGNIIHGNNIWDLTQDGCYVADYYVKTGTDGFVTDKCSSSGNSGVSGTSTSSVGGAAGPMKDDYPYKSNCEGTDPWLYFKCQCTSFVAWRINERLGIKFHNQYKGVNWGDANTWDDAAKRTGVTVNKTPKPGCVAQTDKGTWGHVAWVAAVGDETVTIEEYNYGGKERYGTRTVPKGEFDYIHVKN

SEQ ID NO: 57 - protein, 216 aa, Zopfiella sp
YPIKGDTVNCRSGPGTSFAVKKTYAKGKDIKITCQTTGTSVNGNNIWDKTSDGCYVADYYVKTGKDGFVTDKCDASSVPKPPATKIPGPVTNDYPYKSSCGGVDPWNYYKCQCTSFVAWRVNKRLGKKFHNRYKGHAWGNANQWDEAAKQSGVKVNSTPKPGCIAQTNAGSYGHVAWVTAVGKDTVTIEEYNWNKKLGYGTRTVAKSKFNYIHLEP

SEQ ID NO: 58 - protein, 152 aa, Acremonium exiguum
RSIPIVARAYPGLDDVQSRNAEAALGEIRAEGINPARQACLAVISTALQESELYIYANPAVPESMNYPHDREGGDQDSVGMFQQRPEWYPDIAADMSASGSTRQFLAAMLQVPGWQTMEVSALDQEVQKAEAGNLYGQRIPLATQVCDAAGF

SEQ ID NO: 59 - protein, 217 aa, Chaetomium sp
YPITGDPLNCRSGPGTSYAIKKSYTKGADIKISCQTEGTSVNGNSIWDKTQDGCYVADFYVKTGVDGYVTKKCSGGGGDTGGGSGGIPGPVKDDYPYKSSCGGVDPWNYYKCQCTSFVAWRINKRLGIKFHNQYKGTNWGNANTWDNAAKKTGVKVNSTPKPGAIAQSNAGNYGHVAWVTAVNGNKVTIEEYNWGKREGYGKRTVDKSAFNYIHVKA

(19) United States
(12) Patent Application Publication
(10) Pub. No.: US 2021/0037856 A1
(43) Pub. Date: Feb. 11, 2021

(54) Ruminant Feed Composition Comprising A Muramidase

(71) Applicant: Novozymes A/S, Bagsvaerd (DK)

(72) Inventors: Adam Christian Storm, Bagsvaerd (DK); Lene Venke Kofod, Bagsvaerd (DK)

(73) Assignee: Novozymes A/S, Bagsvaerd (DK)

(21) Appl. No.: 16/978,162

(22) PCT Filed: Mar. 5, 2019
(86) PCT No.: PCT/EP2019/055458

§ 371 Date: Sep. 3, 2020

(30) Foreign Application Priority Data
Mar. 5, 2018 (EP) 18159964.8

(51) Int. Cl. A23K 20/189; A23K 50/10; A23K 20/195; C12N 9/36

(52) U.S. Cl. CPC A23K 20/189; A23K 50/10; C12Y 302/01017; C12N 9/2462; A23K 20/195

(57) ABSTRACT

The present invention relates to methods of improving digestibility in ruminants using ruminant feed comprising polypeptides having muramidase activity

### REFERENCE TO SEQUENCE LISTING

This application contains a Sequence Listing in computer readable form. The computer readable form is incorporated herein by reference.

### FIELD OF THE INVENTION

The present invention relates to methods of improving digestibility in ruminants using ruminant feed comprising polypeptides having muramidase activity.

### BACKGROUND OF THE INVENTION

Muramidase, is a lysozyme, also known as N-acetylmuramide glycanhydrolase, which is an Oglycosyl hydrolase produced as a defensive mechanism against bacteria by many organisms. The enzyme causes the hydrolysis of bacterial cell wall by cleaving the glycosidic bonds of peptidoglycan; an important structural molecule in bacteria. After having their cell wall weakened by muramidase action, bacterial cells lyse as a result of umbalanced osmotic pressure.

Muramidase naturally occurs in many organisms such as viruses, plants, insects, birds, reptiles and mammals. In mammals, Muramidase has been isolated from nasal secretions, saliva, tears, intestinal content, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide of many microorganisms.

Muramidase has been classified into seven different glycoside hydrolase (GH) families (CAZy, www.cazy.org): GH18, GH19, hen egg-white lysozyme (GH22), goose egg-white lysozyme (GH23), bacteriophage T4 muramidase (GH24), Sphingomonas flagellar protein (GH73) and Chalaropsis muramidases (GH25). Muramidases from the families GH23 and GH24 are primarily known from bacteriophages and have only recently been identified in fungi. The muramidase family GH25 has been found to be structurally unrelated to the other muramidase families. Furthermore, an additional class of polypeptides having muramidase activity has been identified in PCT/CN2017/084074, such muramidases are herein called novel MUR polypeptides having muramidase activity, where muramidase activity is defined in the definition section and representative muramidases are listed in the sequence listing.

Muramidase has traditionally been extracted from hen egg white and called hen egg white lysozyme due to its natural abundance. Until very recently hen egg white lysozyme was the only muramidase investigated for use in animal feed. Muramidase extracted from hen egg white is the primary product available on the commercial market, but does not cleave N,6-O-diacetylmuramic acid in e.g. Staphylococcus aureus cell walls and is thus unable to lyse this important human pathogen among others (Masschalck B, Deckers D, Michiels C W (2002), “Lytic and nonlytic mechanism of inactivation of gram-positive bacteria by muramidase under atmospheric and high hydrostatic pressure”, J Food Prot. 65(12):1916-23).

WO2000/21381 discloses a composition comprising at least two antimicrobial enzymes and a polyunsaturated fatty acid, wherein one of the antimicrobial enzymes was a GH22 muramidase from hen egg white. GB2379166 discloses a composition comprising a compound that disrupts the peptidoglycan layer of bacteria and a compound that disrupts the phospholipid layer of bacteria, wherein the peptidoglycan disrupting compound was a GH22 muramidase from hen egg white.

WO2004/026334 discloses an antimicrobial composition for suppressing the growth of enteric pathogens in the gut of livestock comprising (a) a cell wall lysing substance or its salt, (b) a antimicrobial substance, (c) a sequestering agent and (d) an antibiotic, wherein the cell wall lysing substance or its salt is a GH22 muramidase from hen egg white.

The demand for products from ruminants, such as dairy and meat, is increasing which is leading to an increased demand for feed for ruminants. It is an object of the invention to improve the efficiency of nutrient utilisation in feed to reduce the environmental impact of dairy and beef production.

### SUMMARY OF THE INVENTION

The invention provides ruminant feed compositions, such as a ruminant feed, ruminant feed supplements or ruminant feed additives, comprising one or more muramidases wherein the muramidase is in an amount sufficient for administration at a level of 1 to 200 mg enzyme protein per kg ruminant feed.

Further provided is a method for increasing dry matter digestibility of a ruminant feed, ruminant feed supplement or ruminant feed additive comprising the steps of: a) providing at least one muramidase; b) providing a ruminant feed, ruminant feed supplement or ruminant feed additive suitable for a ruminant animal; c) applying the muramidase to the ruminant feed, ruminant feed supplement or ruminant feed additive to form a ruminant feed composition; and d) administering the ruminant feed composition to the ruminant animal, whereby an increase in dry matter digestibility is effected.

In one embodiment of the invention, the production of volatile fatty acids (VFA) in the rumen is increased compared to the production of VFA in the rumen of a ruminant not fed with a muramidase. In a further embodiment, the production of propionate in the rumen is increased compared to the production of propionate in the rumen of a ruminant not fed with a muramidase and/or the production of acetate in the rumen is increased compared to the production of acetate in the rumen of a ruminant not fed with a muramidase.

The muramidase used in the present invention may be of microbial origin. In one embodiment, the muramidase comprises one or more domains from a glycoside hydrolase (GH) family selected from the list consisting of GH24, GH25 and novel MUR polypeptides having muramidase activity.

### BRIEF DESCRIPTION OF DRAWINGS

FIG. 1 shows the effect of 9 muramidases (A: SEQ ID NO: 3, B: SEQ ID NO: 6, C: SEQ ID NO: 9, D: SEQ ID NO: 12, E: SEQ ID NO: 15, F: SEQ ID NO: 18, G: SEQ ID NO: 21, H: SEQ ID NO: 24 and I: SEQ ID NO: 27) from 3 different glycoside hydrolase (GH) families (GH24, GH25 and novel MUR polypeptides having muramidase activity) and positive control (PC, with monensin) on the relative improvement of dry matter digestibility given in percent improvement over control following 48 h of fermentation in ruminal fluid and buffer solution.

FIG. 2 shows the relative difference in ruminal dry matter digestibility compared to negative control as an effect of increasing dosage of muramidase after 12 h of fermentation (NC is negative control, PC is positive control with monensin, A is SEQ ID NO: 28, B is SEQ ID NO: 21, C is SEQ ID NO: 12, D is SEQ ID NO: 18, E is SEQ ID NO: 9).

FIG. 3: shows the relative difference in ruminal acetate production compared to negative control as an effect of increasing dosage of muramidase after 12 h of fermentation (NC is negative control, PC is positive control with monensin, A is SEQ ID NO: 28, B is SEQ ID NO: 21, C is SEQ ID NO: 12, D is SEQ ID NO: 18, E is SEQ ID NO: 9).

FIG. 4: shows the relative difference in ruminal propionate production compared to negative control as an effect of increasing dosage of muramidase after 12 h of fermentation (PC is positive control with monensin, A is SEQ ID NO: 28, B is SEQ ID NO: 21, C is SEQ ID NO: 12, D is SEQ ID NO: 18, E is SEQ ID NO: 9).

FIG. 5: shows the relative difference in ruminal butyrate production compared to negative control as an effect of increasing dosage of muramidase after 12 h of fermentation (PC is positive control with monensin, A is SEQ ID NO: 28, B is SEQ ID NO: 21, C is SEQ ID NO: 12, D is SEQ ID NO: 18, E is SEQ ID NO: 9).

FIG. 6: shows the relative difference in total ruminal VFA production compared to negative control as an effect of increasing dosage of muramidase after 12 h of fermentation (PC is positive control with monensin, A is SEQ ID NO: 28, B is SEQ ID NO: 21, C is SEQ ID NO: 12, D is SEQ ID NO: 18, E is SEQ ID NO: 9).

FIG. 7: shows the relative difference in carbon in total ruminal VFA production compared to negative control as an effect of increasing dosage of muramidase after 12 h of fermentation (PC is positive control with monensin, A is SEQ ID NO: 28, B is SEQ ID NO: 21, C is SEQ ID NO: 12, D is SEQ ID NO: 18, E is SEQ ID NO: 9).

FIG. 8: shows the relative difference in ruminal dry matter digestibility compared to negative control after 12 h of fermentation, as an effect of muramidase and monensin supplementation (NC is negative control, PC is positive control with monensin, A is SEQ ID NO: 28, B is SEQ ID NO: 21, C is SEQ ID NO: 12, D is SEQ ID NO: 29, E is SEQ ID NO: 30, F is SEQ ID NO: 31, G is SEQ ID NO: 32, H is SEQ ID NO: 33, I is SEQ ID NO: 34, J is SEQ ID NO: 35, K is SEQ ID NO: 36, L is SEQ ID NO: 36, M is SEQ ID NO: 37).

FIG. 9: shows the relative difference in ruminal propionate concentration compared to negative control after 12 h of fermentation, as an effect of muramidase and monensin supplementation (NC is negative control, PC is positive control with monensin, A is SEQ ID NO: 28, B is SEQ ID NO: 21, C is SEQ ID NO: 12, D is SEQ ID NO: 29, E is SEQ ID NO: 30, F is SEQ ID NO: 31, G is SEQ ID NO: 32, H is SEQ ID NO: 33, I is SEQ ID NO: 34, J is SEQ ID NO: 35, K is SEQ ID NO: 36, L is SEQ ID NO: 36, M is SEQ ID NO: 37).

FIG. 10 shows the relative difference in ruminal butyrate concentration compared to negative control after 12 h of fermentation, as an effect of muramidase and monensin supplementation (NC is negative control, PC is positive control with monensin, A is SEQ ID NO: 28, B is SEQ ID NO: 21, C is SEQ ID NO: 12, D is SEQ ID NO: 29, E is SEQ ID NO: 30, F is SEQ ID NO: 31, G is SEQ ID NO: 32, H is SEQ ID NO: 33, I is SEQ ID NO: 34, J is SEQ ID NO: 35, K is SEQ ID NO: 36, L is SEQ ID NO: 36, M is SEQ ID NO: 37).

FIG. 11: shows the relative difference in total ruminal VFA production compared to negative control as an effect of muramidase supplementation after 12 h of fermentation (NC is negative control, PC is positive control with monensin, A is SEQ ID NO: 38, B is SEQ ID NO: 39, C is SEQ ID NO: 40, D is SEQ ID NO: 41, E is SEQ ID NO: 42, F is SEQ ID NO: 43, G is SEQ ID NO: 44, H is SEQ ID NO: 45, I is SEQ ID NO: 46, J is SEQ ID NO: 47, K is SEQ ID NO: 48, L is SEQ ID NO: 49, M is SEQ ID NO: 50, N is SEQ ID NO: 51, O is SEQ ID NO: 52, S is SEQ ID NO: 55, T is SEQ ID NO: 56, U is SEQ ID NO: 57, V is SEQ ID NO: 58, W is SEQ ID NO: 59, Y is SEQ ID NO: 53, Z is SEQ ID NO: 54).

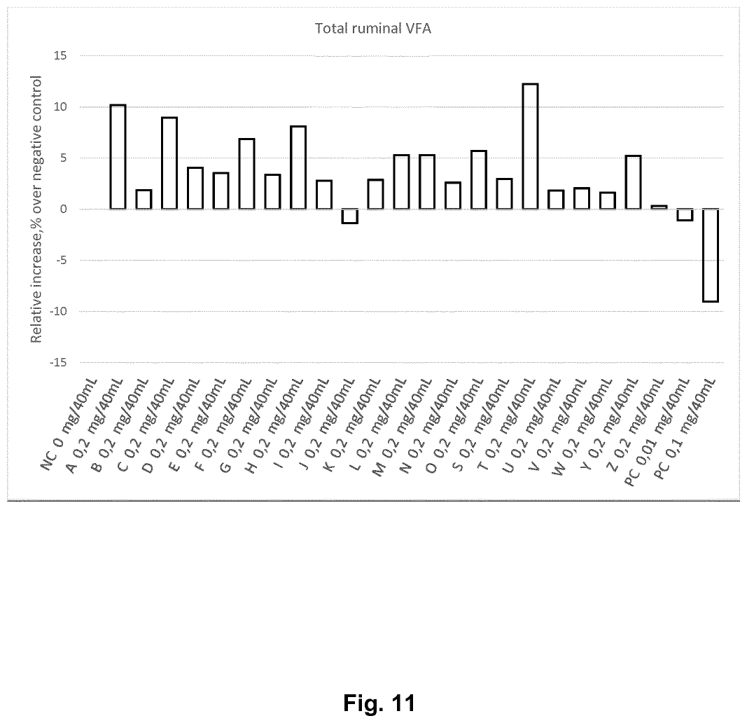

FIG. 12: shows the relative difference in carbon in ruminal VFA production compared to negative control as an effect of muramidase supplementation after 12 h of fermentation (NC is negative control, PC is positive control with monensin, A is SEQ ID NO: 38, B is SEQ ID NO: 39, C is SEQ ID NO: 40, D is SEQ ID NO: 41, E is SEQ ID NO: 42, F is SEQ ID NO: 43, G is SEQ ID NO: 44, H is SEQ ID NO: 45, I is SEQ ID NO: 46, J is SEQ ID NO: 47, K is SEQ ID NO: 48, L is SEQ ID NO: 49, M is SEQ ID NO: 50, N is SEQ ID NO: 51, O is SEQ ID NO: 52, S is SEQ ID NO: 55, T is SEQ ID NO: 56, U is SEQ ID NO: 57, V is SEQ ID NO: 58, W is SEQ ID NO: 59, Y is SEQ ID NO: 53, Z is SEQ ID NO: 54).

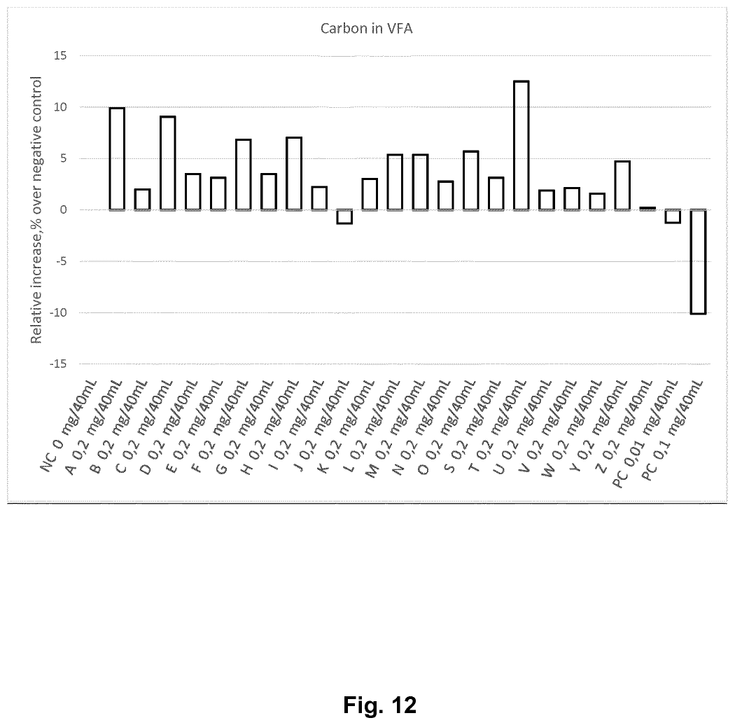

FIG. 13: shows the relative difference in ruminal acetate production compared to negative control as an effect of muramidase supplementation after 12 h of fermentation (NC is negative control, PC is positive control with monensin, A is SEQ ID NO: 38, B is SEQ ID NO: 39, C is SEQ ID NO: 40, D is SEQ ID NO: 41, E is SEQ ID NO: 42, F is SEQ ID NO: 43, G is SEQ ID NO: 44, H is SEQ ID NO: 45, I is SEQ ID NO: 46, J is SEQ ID NO: 47, K is SEQ ID NO: 48, L is SEQ ID NO: 49, M is SEQ ID NO: 50, N is SEQ ID NO: 51, O is SEQ ID NO: 52, S is SEQ ID NO: 55, T is SEQ ID NO: 56, U is SEQ ID NO: 57, V is SEQ ID NO: 58, W is SEQ ID NO: 59, Y is SEQ ID NO: 53, Z is SEQ ID NO: 54).

FIG. 14: shows the relative difference in ruminal propionate production compared to negative control as an effect of muramidase supplementation after 12 h of fermentation (NC is negative control, PC is positive control with monensin, A is SEQ ID NO: 38, B is SEQ ID NO: 39, C is SEQ ID NO: 40, D is SEQ ID NO: 41, E is SEQ ID NO: 42, F is SEQ ID NO: 43, G is SEQ ID NO: 44, H is SEQ ID NO: 45, I is SEQ ID NO: 46, J is SEQ ID NO: 47, K is SEQ ID NO: 48, L is SEQ ID NO: 49, M is SEQ ID NO: 50, N is SEQ ID NO: 51, O is SEQ ID NO: 52, S is SEQ ID NO: 55, T is SEQ ID NO: 56, U is SEQ ID NO: 57, V is SEQ ID NO: 58, W is SEQ ID NO: 59, Y is SEQ ID NO: 53, Z is SEQ ID NO: 54).

### BRIEF DESCRIPTION OF SEQUENCES

SEQ ID NO: 1 is the cDNA sequence of a muramidase polypeptide as isolated from Trichoderma koningiopsis.

SEQ ID NO: 2 is the amino acid sequence as deduced from SEQ ID NO: 1.

SEQ ID NO: 3 is the amino acid sequence of the mature muramidase polypeptide from Trichoderma koningiopsis.

SEQ ID NO: 4 is the cDNA sequence of a muramidase polypeptide as isolated from Thielavia terrestris.

SEQ ID NO: 5 is the amino acid sequence as deduced from SEQ ID NO: 4.

SEQ ID NO: 6 is the amino acid sequence of the mature muramidase polypeptide from Thielavia terrestris.

SEQ ID NO: 7 is the cDNA sequence of a muramidase polypeptide as isolated from Tilletia indica.

SEQ ID NO: 8 is the amino acid sequence as deduced from SEQ ID NO: 7.

SEQ ID NO: 9 is the amino acid sequence of the mature muramidase polypeptide from Tilletia indica.

SEQ ID NO: 10 is the cDNA sequence of a muramidase polypeptide as isolated from Acremonium alcalophilum.

SEQ ID NO: 11 is the amino acid sequence as deduced from SEQ ID NO: 10.

SEQ ID NO: 12 is the amino acid sequence of the mature muramidase polypeptide from Acremonium alcalophilum.

SEQ ID NO: 13 is the cDNA sequence of a muramidase polypeptide as isolated from Cladorrhinum bulbillosum

SEQ ID NO: 14 is the amino acid sequence as deduced from SEQ ID NO: 13.

SEQ ID NO: 15 is the amino acid sequence of the mature muramidase polypeptide from Cladorrhinum bulbillosum.

SEQ ID NO: 16 is the cDNA sequence of a muramidase polypeptide as isolated from Onygena equina.

SEQ ID NO: 17 is the amino acid sequence as deduced from SEQ ID NO: 16.

SEQ ID NO: 18 is the amino acid sequence of the mature muramidase polypeptide from Onygena equina.

SEQ ID NO: 19 is the cDNA sequence of a muramidase polypeptide as isolated from Trichophaea saccata.

SEQ ID NO: 20 is the amino acid sequence as deduced from SEQ ID NO: 19.

SEQ ID NO: 21 is the amino acid sequence of the mature muramidase polypeptide from Trichophaea saccata.

SEQ ID NO: 22 is the cDNA sequence of a muramidase polypeptide as isolated from Pleurotus ostreatus.

SEQ ID NO: 23 is the amino acid sequence as deduced from SEQ ID NO: 22.

SEQ ID NO: 24 is the amino acid sequence of the mature muramidase polypeptide from Pleurotus ostreatus.

SEQ ID NO: 25 is the cDNA sequence of a muramidase polypeptide as isolated from Cladosporium sp-9768.

SEQ ID NO: 26 is the amino acid sequence as deduced from SEQ ID NO: 25.

SEQ ID NO: 27 is the amino acid sequence of the mature muramidase polypeptide from Cladosporium sp-9768.

SEQ ID NO: 28 is the amino acid sequence of the mature muramidase polypeptide from Chaetomium thermophilum var. thermophilum.

SEQ ID NO: 29 is the amino acid sequence of the mature muramidase polypeptide from Acremonium alcalophilum.

SEQ ID NO: 30 is the amino acid sequence of the mature muramidase polypeptide from Coprinopsis cinerea okayama.

SEQ ID NO: 31 is the amino acid sequence of the mature muramidase polypeptide from Rasamsonia brevistipitata.

SEQ ID NO: 32 is the amino acid sequence of the mature muramidase polypeptide from Acremonium alcalophilum.

SEQ ID NO: 33 is the amino acid sequence of the mature muramidase polypeptide from Poronia punctata.

SEQ ID NO: 34 is the amino acid sequence of the mature muramidase polypeptide from Aspergillus deflectus.

SEQ ID NO: 35 is the amino acid sequence of the mature muramidase polypeptide from Poronia punctata.

SEQ ID NO: 36 is the amino acid sequence of the mature muramidase polypeptide from Paecilomyces sp.

SEQ ID NO: 37 is the amino acid sequence of the mature muramidase polypeptide from Hamigera sp.

SEQ ID NO: 38 is the amino acid sequence of the mature muramidase polypeptide from Penicillium citrinum.

SEQ ID NO: 39 is the amino acid sequence of the mature muramidase polypeptide from Pyronema domesticum.

SEQ ID NO: 40 is the amino acid sequence of the mature muramidase polypeptide from Thielavia sp.

SEQ ID NO: 41 is the amino acid sequence of the mature muramidase polypeptide from Chaetomium sp.

SEQ ID NO: 42 is the amino acid sequence of the mature muramidase polypeptide from Metarhizium iadini.

SEQ ID NO: 43 is the amino acid sequence of the mature muramidase polypeptide from Aspergillus deflectus.

SEQ ID NO: 44 is the amino acid sequence of the mature muramidase polypeptide from Sporormia fimetaria.

SEQ ID NO: 45 is the amino acid sequence of the mature muramidase polypeptide from Lecanicillium psaffiotae.

SEQ ID NO: 46 is the amino acid sequence of the mature muramidase polypeptide from Trichocladium asperum.

SEQ ID NO: 47 is the amino acid sequence of the mature muramidase polypeptide from Clavicipitaceae sp-70249.

SEQ ID NO: 48 is the amino acid sequence of the mature muramidase polypeptide from Thielavia terrestris.

SEQ ID NO: 49 is the amino acid sequence of the mature muramidase polypeptide from Westerdykella.

SEQ ID NO: 50 is the amino acid sequence of the mature muramidase polypeptide from Onygena equina.

SEQ ID NO: 51 is the amino acid sequence of the mature muramidase polypeptide from Ovatospora brasiliensis.

SEQ ID NO: 52 is the amino acid sequence of the mature muramidase polypeptide from Purpureocillium lilacinum.

SEQ ID NO: 53 is the amino acid sequence of the mature muramidase polypeptide from Ovatospora brasiliensis.

SEQ ID NO: 54 is the amino acid sequence of the mature muramidase polypeptide from Penicillium wellingtonense.

SEQ ID NO: 55 is the amino acid sequence of the mature muramidase polypeptide from Aspergffius sp.

SEQ ID NO: 56 is the amino acid sequence of the mature muramidase polypeptide from Chaetomium sp.

SEQ ID NO: 57 is the amino acid sequence of the mature muramidase polypeptide from Zopfiella Sp.

SEQ ID NO: 58 is the amino acid sequence of the mature muramidase polypeptide from Acremonium exiguum.

SEQ ID NO: 59 is the amino acid sequence of the mature muramidase polypeptide from Chaetomium sp.

### Definitions

Acetate: Acetate is herein used interchangeably with the term “acetic acid” and is one of the volatile fatty acids (VFA) produced in the rumen. It is a precursor for mammalian milk fat synthesis and is also used for muscle energy metabolism and body fat synthesis. The amount of acetate in the rumen is a measure of rumen fermentation of the ingested feed, an increase in ruminal acetate is thus an indication of increased energy supply for ruminants.

Antimicrobial activity: The term “antimicrobial activity” is defined herein as an activity that kills or inhibits the growth of microorganisms, such as, algae, archea, bacteria, fungi and/or protozoans. The antimicrobial activity can, for example, be bactericidal meaning the killing of bacteria or bacteriostatic meaning the prevention of bacterial growth. The antimicrobial activity can include catalysing the hydrolysis of 1,4-beta-linkages between N-acetylmuramic acid and N-acetyl-D-glucosamine residues in a peptidoglycan and between N-acetyl-D-glucosamine residues in chitodextrins. Antimicrobial activity can also include the muramidase binding to the surface of the microorganism and inhibiting its growth. The antimicrobial effect can also include the use of the muramidases of the present invention for activation of bacterial autolysins, as an immunostimulator, by inhibiting or reducing bacterial toxins and by an opsonin effect.

Beef production: The term “beef production” is defined herein as the production of beef from cattle raised for meat production. Beef production may e.g. be measured by feed intake, daily feed intake, body weight gain, average daily gain, carcass dressing present, carcass composition, and carcass scoring.

Butyrate: “Butyrate” is herein used interchangeably with the term “butyric acid” and is one of the volatile fatty acids (VFA) produced in the rumen. It is a precursor of β-OH-butyrate that is used for mammalian milk fat synthesis and is also used for muscle energy metabolism and body fat synthesis. The amount of butyrate in the rumen is a measure of rumen fermentation of the ingested feed, an increase in butyrate is thus an indication of increased energy supply for ruminants.

Concentrates: The term “concentrates” means feed with high and rapid Dry Matter digestibility (DMd). Typically concentrates are feed stuffs with relative high protein and/or energy concentrations and low in Nutrient Detergent Fibre (NDF) concentration, such as molasses, oligosaccharides, sorghum, seeds and grains (either whole or prepared by crushing, milling, etc. from e.g. corn, oats, rye, barley, wheat), oilseed press cake, oilseed press meal (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Dry Matter digestibility (DMd): Digestibility refers to the extent to which a feedstuff is degraded and absorbed into the body of an animal while passing through the digestive tract. The term “Dry Matter digestibility” means the disappearance of feed dry matter from the gastrointestinal (GI) tract by a given animal at a specified level of feed intake. DMd is measured as the percentage difference in dry matter (DM) proportion between ingested feed and excreted feces coming from the ingested feed. Ruminal DMd is thus the percentage difference in dry matter proportion between ingested feed and digesta passed to the distal compartments of the rumen, and describes the potential for the ruminal microbes to backdown and digest the feed DM.

Energy Corrected Milk (ECM): “Energy corrected milk” is a means of adjusting the milk yield for the amount of major components in milk that affect the energy concentration (lactose, fat and protein) in the milk, and determines the amount of milk produced adjusted to 3.5 percent fat and 3.2 percent protein. ECM is herein calculated as described by Sjaunja, L. O., Baevre, L., Junkkarinen, L., Pedersen, J., Setala, J. “A Nordic proposal for an energy corrected milk (ECM) formula” in: P. Gaillon, Y. Chabert (Eds.) “Performance Recording of Animals: State of the Art, 1990”: Proceedings of the 27th Biennial Session of the International Committee for Animal Recording. Wageningen Academic Publishers, Wageningen, the Netherlands; 1991:156-157.

Feed Conversion Ratio (FCR): FCR is a measure of an animal's (herein a ruminant) efficiency in converting feed mass into the desired output e.g. body mass. FCR is calculated as feed intake divided by weight gain of the animal, all over a specified period. By “lower feed conversion ratio” or “improved feed conversion ratio” it is meant that less feed is required to increase the weight of the animal and/or the milk production of the animal. A FCR improvement of 2% means that the FCR was reduced by 2%.

Feed efficiency: The term “feed efficiency” is the ratio of live-weight gain to dry matter intake (DMI), or the Energy Corrected Milk production per kg of dry matter intake (kg ECM/kg DMI). The higher the number the better.

Forage: The term “forage” as defined herein also includes roughage. Forage is NDF rich plant material such as hay and silage from forage plants, grass and other forage plants, seaweed, and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), whole crop products using corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes.

Fungal muramidase: The term “fungal muramidase” means a polypeptide having muramidase activity which is obtained or obtainable from a fungal source. Examples of fungal sources are fungi; i.e. the muramidase is obtained or obtainable from the kingdom Fungi, wherein the term kingdom is the taxonomic rank. In particular, the fungal muramidase is obtained or obtainable from the phylum Ascomycota, such as the sub-phylum Pezizomycotina, wherein the terms phylum and sub-phylum is the taxonomic ranks.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Ionophore: The term “ionophore” is herein used for antibiotics, e.g. macrolide antibiotics, and/or growth enhancing feed additives for animals such as ruminants, which catalyze ion transport across hydrophobic membranes such as lipid bilayers found in the living cells and exhibit high affinities for ions, such as e.g. Na⁺, H⁻, Ca²⁺, Mg²⁺ and/or K. Examples of ionophores include without limitations Monensin, which is e.g. used in the beef and dairy industries to prevent coccidiosis, increase the production of propionic acid and prevent bloat.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc.

Milk production: The term “milk production” is used for describing the entire production of milk from dairy cattle. Milk production is measured in total amount of milk produced, it can be expressed as daily milk production or milk production per lactation defined as the period from the day of calving to the day of dry off defined as the day the cow stops giving milk. The day of dry off is typically around 300 days after calving. Milk production is measured in Kg milk or Kg energy corrected milk (ECM) to compensate for the variation in milk solid.

Muramidase: The term “muramidase” is used for polypeptides having glycoside hydrolase activity and catalyze the hydrolysis of 1,4-beta-linkages between N-acetylmuramic acid and N-acetyl-D-glucosamine residues in peptidoglycan. This hydrolysis in turn compromises the integrity of bacterial cell walls causing lysis of the bacteria. Other terms for muramidase include “lysozyme” and “N-acetylmuramide glycanhydrolase”.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)).

Organic Matter digestibility (OMd): Digestibility of organic matter is defined as DMd but where the amount of organic matter is calculated as: OM=DM-ash, were the ash content is determined after total combustion of the feed DM.

Propionate: “Propionate” is herein used interchangeably with the term “propionic acid” and is one of the volatile fatty acids (VFA) produced in the rumen. Propionate is the main precursor for glucose synthesis by ruminants, glucose is used for lactose and energy metabolism. The amount of propionate in the rumen is a measure of rumen fermentation of the ingested feed, an increase in ruminal propionate is thus an indication of increased glucose supply for ruminants.

Ruminant: The term “ruminant” means a mammal that digests plant-based feed by initially fermenting/degrading it within the animal's first compartment of the forestomach complex, principally through bacterial actions, then retaining small particles and regurgitating long semi-degraded mass, now known as cud, and chewing it again. The process of re-chewing the cud to further break down plant matter and stimulate digestion is called “ruminating”. Examples of ruminants are cattle, cow, dairy cattle, beef cattle, buffalo, calf, goat, sheep, lamb, deer, reindeer, yak, camel and llama.

Ruminant feed: The term “ruminant feed” or “animal feed for ruminants” refers to any compound, preparation, or mixture suitable for, or intended for intake by a ruminant. Ruminant feed typically comprises forages (including fresh grass, roughage and silage) and may further comprise of concentrates as well as vitamins, minerals, enzymes, direct fed microbial (DFM), amino acid and/or other feed ingredients (such as in a premix). Ruminant feed can be fed as total mixed ration (TMR) where all feed components are mixed together before feeding and fed as one mixture, or as partly mixed ration (PMR) where most of the feed components are mixed and fed together but some of the concentrate is fed separately or it can be fed as separately fed feed, were all components are fed separately without mixing.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS:

The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

Silage: Silage is a type of forage that is produced from natural fermentation of wet plant material, such as fresh grass, and whole crops e.g. corn and barley. The fermentation process is performed to preserve the wet material so it can be used throughout the year.

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having muramidase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a muramidase variant according to the invention may comprise from 1 to 5; from 1 to 10; from 1 to 15; from 1 to 20; from 1 to 25; from 1 to 30; from 1 to 35; from 1 to 40; from 1 to 45; or from 1-50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59.

Volatile fatty acids (VFA)/short-chain fatty acids (SCFA): Volatile fatty acids (VFA), also referred to as short-chain fatty acids (SOFA), are fatty acids with less than six carbon atoms and e.g. include acetic acid, propionic acid, butyric acid, isobutyric acid, valeric acid and isovaleric acid. Volatile fatty acids (VFA) are produced from fermentation of carbohydrates in the rumen and provide the main energy source in ruminants. Increase in VFA can thus be used as an indication of increase in energy and nutrient supply for ruminants.

### DETAILED DESCRIPTION OF THE INVENTION

### Methods of Improving Ruminant Performance

The nutrient utilization of feed for ruminants is important for optimal production and animal health in modern production systems. It has surprisingly been found that supplementing a ruminant feed with a muramidase according to the invention results in increased ruminal dry matter digestibility compared to when supplementing ruminant feed without the muramidase (as control). By increasing the ruminal dry matter digestibility, the ruminants are provided with more nutrients for production.

The efficiency of nutrient utilization has thus been improved and conversion of organic matter, such as e.g. microbial protein into milk by the dairy cattle or to meat by the beef cattle, has increased compared to the conversion obtained by ruminant feed without muramidase.

It has furthermore surprisingly been found that the ruminal dry matter digestibility is improved compared to the ruminal dry matter digestibility obtained when providing ionophores commonly used in the market to the ruminant.

In one aspect of the invention, efficiency of nutrient utilization of the ruminant feed is increased. By increasing the nutrient utilisation of feed for ruminants, the same amount of milk and/or meat can be produced from less ruminant animals, reducing natural resource use and greenhouse gas (GHG) emissions per unit of milk and/or unit of meat produced. It also leads to decreased nitrogen and phosphate excretion per ruminant animal and therefore a total reduction in phosphate and nitrogen excretion per unit of production.

Determination of dry matter digestibility may e.g. be performed using an in vitro fermentation model adapted from Menke K H, Steingass H. 1988 (Estimation of the energetic feed value obtained from chemical analysis and in vitro gas production using rumen fluid. Anim Res Dev. (1988) 28:7-55) as described in example 1.

In one aspect, the production of volatile fatty acids (VFA) in the rumen are increased compared to the VFA produced in the rumen of a ruminant not fed with a muramidase.

In one aspect, the production of propionate in the rumen is increased compared to propionate produced in the rumen of a ruminant not fed with a muramidase.

In one aspect, the production of acetate in the rumen is increased compared to the production of acetate in the rumen of a ruminant not fed with a muramidase.

In one embodiment, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed DM, or any combination of these intervals.

In one embodiment, the ruminant is selected from the group consisting of: cattle, cow, dairy cattle, beef cattle, buffalo, young calf, goat, sheep, lamb, deer, yak, camel and llama. In a further embodiment, the ruminant is selected from the group consisting of cattle, dairy cattle and beef cattle.

The muramidase may be provided to the ruminant during any period of time from birth until slaughter. In a preferred embodiment the muramidase is provided to the ruminant on a daily basis. In a further embodiment, the muramidase is provided to the ruminant on a daily basis during the lifespan of the ruminant.

In one embodiment, the muramidase is provided to ruminants selected from the group consisting of: cattle, cow, dairy cattle, beef cattle, buffalo, calf, goat, sheep, lamb, deer, yak, camel and llama. In one embodiment, the muramidase is provided to growing ruminants. In one embodiment, the muramidase is provided to dairy cattle. In a further embodiment, the muramidase is provided to dairy cattle during lactation. In one embodiment, the muramidase is provided to beef cattle in the growing phase of beef cattle production. In one embodiment, the muramidase is provided to beef cattle in the finishing phase of beef cattle production.

The muramidase may be provided to the ruminant in any suitable way. In one embodiment, the muramidase is fed to the ruminant in a feed, a feed supplement or a feed additive. In another embodiment, the muramidase is provided to the ruminant in the drinking water. In yet another embodiment, the muramidase is provided to the ruminant as bolus administration. In still another embodiment, the muramidase is provided to the ruminant as a post feed spray application applied to the ruminant feed. In one embodiment, the muramidase is provided to the ruminant in liquid form as a drink. In another embodiment, the muramidase is provided to the ruminant in liquid form as a drench. In another embodiment, the muramidase is provided to the ruminant in milk or a milk replacer.

In one embodiment, the muramidase is of microbial origin. In a further embodiment, the muramidase is of fungal origin. In an embodiment, the muramidase is obtained or obtainable from the phylum Ascomycota, such as the sub-phylum Pezizomycotina.

In one embodiment, the muramidase comprises one or more domains from a glycoside hydrolase (GH) family selected from the list consisting of GH24, GH25 and novel MUR polypeptides having muramidase activity.

In one embodiment, the muramidase comprises one or more domains from the glycoside hydrolase (GH) family GH24.

In one embodiment, the muramidase comprises one or more domains from the glycoside hydrolase (GH) family GH25.

In one embodiment, the muramidase comprises one or more domains from the novel MUR polypeptides having muramidase activity.

In a preferred embodiment, the invention relates to a method of improving the Dry Matter digestibility (DMd) and/or volatile fatty acid (VFA) production and/or meat production and/or milk production of a ruminant comprising administering a ruminant feed, ruminant feed supplement or ruminant feed additive comprising one or more muramidases to the ruminant, wherein:

(a) the muramidase is a muramidase comprising one or more domains from a glycoside hydrolase (GH) family selected from the list consisting of GH24, GH25 and novel MUR polypeptides having muramidase activity, and is dosed at a level of 1 to 200 mg enzyme protein per kg ruminant feed DM;
(b) the ruminant is selected from the group consisting of: cattle, cow, dairy cattle, beef cattle, buffalo, calf, goat, sheep, lamb, deer, yak, camel and llama; and
(c) the Dry Matter digestibility (DMd) and/or volatile fatty acid (VFA) production and/or meat production and/or milk production is improved by at least 1% compared to control; and
(d) optionally the muramidase is provided to the ruminant on a daily basis for at least 30 days during the life span of the ruminant.

In one embodiment of the method, the energy corrected milk (ECM) production is improved by at least 1.25%, such as by at least 1.5%, at least 1.75% or at least 2.0% compared to the control. In another embodiment, the ECM production is improved by between 1% and 5%, such as between 1% and 4%, between 1% and 3%, between 1.25% and 2.5%, or between 1.5% and 2% compared to the control, or any combination of these intervals.

In one embodiment of the method, the ruminal dry matter digestibility (DMd) is improved by at least 1%, such as by at least 1.5%, at least 1.75% or at least 2.0% compared to the control. In another embodiment, the dry matter digestibility is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is selected from acetate, propionate, butyrate, isobutyrate, valerate, isovalerate and any combination thereof. In a further embodiment, the volatile fatty acid (VFA) is selected from acetate, propionate, butyrate and any combination thereof. In a yet further embodiment, the volatile fatty acid (VFA) is acetate and/or propionate.

In one embodiment of the method, the volatile fatty acid (VFA) is improved by at least 1%, such as by at least 1.5%, at least 1.75% or at least 2.0% compared to the control. In another embodiment, the volatile fatty acid is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is acetate. In a further embodiment, acetate is improved by at least 1%, such as by at least 1.5%, at least 1.75% or at least 2.0% compared to the control. In another embodiment, acetate is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is propionate. In a further embodiment, propionate is improved by at least 1%, such as by at least 1.5%, at least 1.75% or at least 2.0% compared to the control. In another embodiment, propionate is improved by between 1% and 15%, such as between 1% and 10%, between 1% and 7%, 1% and 5%, or 2% and 5% compared to the control, or any combination of these intervals.

In one embodiment of the method, the FCR is improved by at least 1%, such as by at least 1.25%, at least 1.5%, at least 1.75% or at least 2.0% compared to the control. In another embodiment, the FCR is improved by between 1% and 10%, such as between 1% and 9%, such as between 1% and 8%, such as between 1% and 7%, such as between 1% and 6%, such as between 1% and 5%, such as between 1% and 4%, between 1% and 3%, between 1.25% and 2.5%, or between 1.5% and 2% compared to the control, or any combination of these intervals. A 1% improvement in FCR is defined as 1/100 reduction in the FCR of the ruminant supplemented with muramidase compared to the FCR of the ruminant not supplemented with muramidase.

In one embodiment, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed, or any combination of these intervals.

In one embodiment, the muramidase is provided to the ruminant during any period of time from birth until slaughter. In a preferred embodiment the muramidase is provided to the ruminant on a daily basis. In a further embodiment, the muramidase is provided to the ruminant on a daily basis during life span of the ruminant.

In one embodiment, the muramidase is provided to growing ruminants. In one embodiment, the muramidase is provided to dairy cattle. In a further embodiment, the muramiase is provided to dairy cattle during lactation. In one embodiment, the muramidase is provided to beef cattle in the growing phase of beef cattle production. In one embodiment, the muramidase is provided to beef cattle in the finishing phase of beef cattle production. In a further embodiment, the muramidase is provided to calves in the milk. In one embodiment, the muramidase is provided to ruminants selected from the group consisting of: cattle, cow, dairy cattle, beef cattle, buffalo, calf, goat, sheep, lamb, deer, yak, camel and llama.

In one embodiment, the muramidase is of microbial origin. In a further embodiment, the muramidase is of fungal origin. In an embodiment, the muramidase is obtained or obtainable from the phylum Ascomycota, such as the sub-phylum Pezizomycotina.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3.

In one embodiment, the muramidase comprises or consists of the amino acid sequence of SEQ ID NO: 3 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 3 or an allelic variant thereof and a N-terminal and/or C-terminal His-tag and/or HQ-tag. In another aspect, the polypeptide comprises or consists of amino acids 1 to 213 of SEQ ID NO: 3.

In another embodiment, the muramidase is a variant of SEQ ID NO: 3 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 3 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 3 is not more than 10, e.g., 1, 2, 3, 4, 5, 6, 7, 8, 9 or 10. In another embodiment, the number of substitutions, deletions, and/or insertions in SEQ ID NO: 3 is not more than 10, e.g., 1, 2, 3, 4, 5, 6, 7, 8, 9 or 10. In a further embodiment, the number of substitutions, preferably conservative substitutions, in SEQ ID NO: 3 is not more than 10, e.g., 1, 2, 3, 4, 5, 6, 7, 8, 9 or 10. In a further embodiment, the number of conservative substitutions in SEQ ID NO: 3 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6.

In one embodiment, the muramidase comprises or consists of the amino acid sequence of SEQ ID NO: 6 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 6 or an allelic variant thereof and a N-terminal and/or C-terminal His-tag and/or HQ-tag. In another aspect, the polypeptide comprises or consists of amino acids 1 to 213 of SEQ ID NO: 6.

In another embodiment, the muramidase is a variant of SEQ ID NO: 6 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 6 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 6 is not more than 10, e.g., 1, 2, 3, 4, 5, 6, 7, 8, 9 or 10. In another embodiment, the number of substitutions, deletions, and/or insertions in SEQ ID NO: 6 is not more than 10, e.g., 1, 2, 3, 4, 5, 6, 7, 8, 9 or 10. In a further embodiment, the number of substitutions, preferably conservative substitutions, in SEQ ID NO: 6 is not more than 10, e.g., 1, 2, 3, 4, 5, 6, 7, 8, 9 or 10. In a further embodiment, the number of conservative substitutions in SEQ ID NO: 6 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 9.

In one embodiment, the muramidase comprises or consists of the amino acid sequence of SEQ ID NO: 9 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 9 or an allelic variant thereof and a N-terminal and/or C-terminal His-tag and/or HQ-tag. In another aspect, the polypeptide comprises or consists of amino acids 1 to 213 of SEQ ID NO: 9.

In another embodiment, the muramidase is a variant of SEQ ID NO: 9 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 9 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 9 is not more than 10, e.g., 1, 2, 3, 4, 5, 6, 7, 8, 9 or 10. In another embodiment, the number of substitutions, deletions, and/or insertions in SEQ ID NO: 9 is not more than 10, e.g., 1, 2, 3, 4, 5, 6, 7, 8, 9 or 10. In a further embodiment, the number of substitutions, preferably conservative substitutions, in SEQ ID NO: 9 is not more than 10, e.g., 1, 2, 3, 4, 5, 6, 7, 8, 9 or 10. In a further embodiment, the number of conservative substitutions in SEQ ID NO: 9 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12.

In one embodiment, the muramidase comprises or consists of the amino acid sequence of SEQ ID NO: 12 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 12 or an allelic variant thereof and a N-terminal and/or C-terminal His-tag and/or HQ-tag. In another aspect, the polypeptide comprises or consists of amino acids 1 to 213 of SEQ ID NO: 12.

In another embodiment, the muramidase is a variant of SEQ ID NO: 12 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 12 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 12 is not more than 10, e.g., 1, 2, 3, 4, 5, 6, 7, 8, 9 or 10. In another embodiment, the number of substitutions, deletions, and/or insertions in SEQ ID NO: 12 is not more than 10, e.g., 1, 2, 3, 4, 5, 6, 7, 8, 9 or 10. In a further embodiment, the number of substitutions, preferably conservative substitutions, in SEQ ID NO: 12 is not more than 10, e.g., 1, 2, 3, 4, 5, 6, 7, 8, 9 or 10. In a further embodiment, the number of conservative substitutions in SEQ ID NO: 12 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15.

In one embodiment, the muramidase comprises or consists of the amino acid sequence of SEQ ID NO: 15 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 15 or an allelic variant thereof and a N-terminal and/or C-terminal His-tag and/or HQ-tag. In another aspect, the polypeptide comprises or consists of amino acids 1 to 213 of SEQ ID NO: 15.

In another embodiment, the muramidase is a variant of SEQ ID NO: 15 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 15 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 15 is not more than 10, e.g., 1, 2, 3, 4, 5, 6, 7, 8, 9 or 10. In another embodiment, the number of substitutions, deletions, and/or insertions in SEQ ID NO: 15 is not more than 10, e.g., 1, 2, 3, 4, 5, 6, 7, 8, 9 or 10. In a further embodiment, the number of substitutions, preferably conservative substitutions, in SEQ ID NO: 15 is not more than 10, e.g., 1, 2, 3, 4, 5, 6, 7, 8, 9 or 10. In a further embodiment, the number of conservative substitutions in SEQ ID NO: 15 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 18.

In one embodiment, the muramidase comprises or consists of the amino acid sequence of SEQ ID NO: 18 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 18 or an allelic variant thereof and a N-terminal and/or C-terminal His-tag and/or HQ-tag. In another aspect, the polypeptide comprises or consists of amino acids 1 to 213 of SEQ ID NO: 18.

In another embodiment, the muramidase is a variant of SEQ ID NO: 18 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 18 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 18 is not more than 10, e.g., 1, 2, 3, 4, 5, 6, 7, 8, 9 or 10. In another embodiment, the number of substitutions, deletions, and/or insertions in SEQ ID NO: 18 is not more than 10, e.g., 1, 2, 3, 4, 5, 6, 7, 8, 9 or 10. In a further embodiment, the number of substitutions, preferably conservative substitutions, in SEQ ID NO: 18 is not more than 10, e.g., 1, 2, 3, 4, 5, 6, 7, 8, 9 or 10. In a further embodiment, the number of conservative substitutions in SEQ ID NO: 18 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21.

In one embodiment, the muramidase comprises or consists of the amino acid sequence of SEQ ID NO: 21 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 21 or an allelic variant thereof and a N-terminal and/or C-terminal His-tag and/or HQ-tag. In another aspect, the polypeptide comprises or consists of amino acids 1 to 213 of SEQ ID NO: 21.

In another embodiment, the muramidase is a variant of SEQ ID NO: 21 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 21 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 21 is not more than 10, e.g., 1, 2, 3, 4, 5, 6, 7, 8, 9 or 10. In another embodiment, the number of substitutions, deletions, and/or insertions in SEQ ID NO: 21 is not more than 10, e.g., 1, 2, 3, 4, 5, 6, 7, 8, 9 or 10. In a further embodiment, the number of substitutions, preferably conservative substitutions, in SEQ ID NO: 21 is not more than 10, e.g., 1, 2, 3, 4, 5, 6, 7, 8, 9 or 10. In a further embodiment, the number of conservative substitutions in SEQ ID NO: 21 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24.

In one embodiment, the muramidase comprises or consists of the amino acid sequence of SEQ ID NO: 24 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 24 or an allelic variant thereof and a N-terminal and/or C-terminal His-tag and/or HQ-tag. In another aspect, the polypeptide comprises or consists of amino acids 1 to 213 of SEQ ID NO: 24.

In another embodiment, the muramidase is a variant of SEQ ID NO: 24 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 24 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 24 is not more than 10, e.g., 1, 2, 3, 4, 5, 6, 7, 8, 9 or 10. In another embodiment, the number of substitutions, deletions, and/or insertions in SEQ ID NO: 24 is not more than 10, e.g., 1, 2, 3, 4, 5, 6, 7, 8, 9 or 10. In a further embodiment, the number of substitutions, preferably conservative substitutions, in SEQ ID NO: 24 is not more than 10, e.g., 1, 2, 3, 4, 5, 6, 7, 8, 9 or 10. In a further embodiment, the number of conservative substitutions in SEQ ID NO: 24 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 27.

In one embodiment, the muramidase comprises or consists of the amino acid sequence of SEQ ID NO: 27 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 27 or an allelic variant thereof and a N-terminal and/or C-terminal His-tag and/or HQ-tag. In another aspect, the polypeptide comprises or consists of amino acids 1 to 213 of SEQ ID NO: 27.

In another embodiment, the muramidase is a variant of SEQ ID NO: 27 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 27 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 27 is not more than 10, e.g., 1, 2, 3, 4, 5, 6, 7, 8, 9 or 10. In another embodiment, the number of substitutions, deletions, and/or insertions in SEQ ID NO: 27 is not more than 10, e.g., 1, 2, 3, 4, 5, 6, 7, 8, 9 or 10. In a further embodiment, the number of substitutions, preferably conservative substitutions, in SEQ ID NO: 27 is not more than 10, e.g., 1, 2, 3, 4, 5, 6, 7, 8, 9 or 10. In a further embodiment, the number of conservative substitutions in SEQ ID NO: 27 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 28.

In one embodiment, the muramidase comprises or consists of the amino acid sequence of SEQ ID NO: 28 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 28 or an allelic variant thereof and a N-terminal and/or C-terminal His-tag and/or HQ-tag. In another aspect, the polypeptide comprises or consists of amino acids 1 to 213 of SEQ ID NO: 28.

In another embodiment, the muramidase is a variant of SEQ ID NO: 28 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 28 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 28 is not more than 10, e.g., 1, 2, 3, 4, 5, 6, 7, 8, 9 or 10. In another embodiment, the number of substitutions, deletions, and/or insertions in SEQ ID NO: 28 is not more than 10, e.g., 1, 2, 3, 4, 5, 6, 7, 8, 9 or 10. In a further embodiment, the number of substitutions, preferably conservative substitutions, in SEQ ID NO: 28 is not more than 10, e.g., 1, 2, 3, 4, 5, 6, 7, 8, 9 or 10. In a further embodiment, the number of conservative substitutions in SEQ ID NO: 28 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 29.

In one embodiment, the muramidase comprises or consists of the amino acid sequence of SEQ ID NO: 29 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 29 or an allelic variant thereof and a N-terminal and/or C-terminal His-tag and/or HQ-tag. In another aspect, the polypeptide comprises or consists of amino acids 1 to 213 of SEQ ID NO: 29.

In another embodiment, the muramidase is a variant of SEQ ID NO: 29 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 29 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 29 is not more than 10, e.g., 1, 2, 3, 4, 5, 6, 7, 8, 9 or 10. In another embodiment, the number of substitutions, deletions, and/or insertions in SEQ ID NO: 29 is not more than 10, e.g., 1, 2, 3, 4, 5, 6, 7, 8, 9 or 10. In a further embodiment, the number of substitutions, preferably conservative substitutions, in SEQ ID NO: 29 is not more than 10, e.g., 1, 2, 3, 4, 5, 6, 7, 8, 9 or 10. In a further embodiment, the number of conservative substitutions in SEQ ID NO: 29 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 30.

In one embodiment, the muramidase comprises or consists of the amino acid sequence of SEQ ID NO: 30 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 30 or an allelic variant thereof and a N-terminal and/or C-terminal His-tag and/or HQ-tag. In another aspect, the polypeptide comprises or consists of amino acids 1 to 213 of SEQ ID NO: 30.

In another embodiment, the muramidase is a variant of SEQ ID NO: 30 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 30 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 30 is not more than 10, e.g., 1, 2, 3, 4, 5, 6, 7, 8, 9 or 10. In another embodiment, the number of substitutions, deletions, and/or insertions in SEQ ID NO: 30 is not more than 10, e.g., 1, 2, 3, 4, 5, 6, 7, 8, 9 or 10. In a further embodiment, the number of substitutions, preferably conservative substitutions, in SEQ ID NO: 30 is not more than 10, e.g., 1, 2, 3, 4, 5, 6, 7, 8, 9 or 10. In a further embodiment, the number of conservative substitutions in SEQ ID NO: 30 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 31.

In one embodiment, the muramidase comprises or consists of the amino acid sequence of SEQ ID NO: 31 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 31 or an allelic variant thereof and a N-terminal and/or C-terminal His-tag and/or HQ-tag. In another aspect, the polypeptide comprises or consists of amino acids 1 to 213 of SEQ ID NO: 31.

In another embodiment, the muramidase is a variant of SEQ ID NO: 31 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 31 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 31 is not more than 10, e.g., 1, 2, 3, 4, 5, 6, 7, 8, 9 or 10. In another embodiment, the number of substitutions, deletions, and/or insertions in SEQ ID NO: 31 is not more than 10, e.g., 1, 2, 3, 4, 5, 6, 7, 8, 9 or 10. In a further embodiment, the number of substitutions, preferably conservative substitutions, in SEQ ID NO: 31 is not more than 10, e.g., 1, 2, 3, 4, 5, 6, 7, 8, 9 or 10. In a further embodiment, the number of conservative substitutions in SEQ ID NO: 31 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 32.

In one embodiment, the muramidase comprises or consists of the amino acid sequence of SEQ ID NO: 32 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 32 or an allelic variant thereof and a N-terminal and/or C-terminal His-tag and/or HQ-tag. In another aspect, the polypeptide comprises or consists of amino acids 1 to 213 of SEQ ID NO: 32.

In another embodiment, the muramidase is a variant of SEQ ID NO: 32 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 32 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 32 is not more than 10, e.g., 1, 2, 3, 4, 5, 6, 7, 8, 9 or 10. In another embodiment, the number of substitutions, deletions, and/or insertions in SEQ ID NO: 32 is not more than 10, e.g., 1, 2, 3, 4, 5, 6, 7, 8, 9 or 10. In a further embodiment, the number of substitutions, preferably conservative substitutions, in SEQ ID NO: 32 is not more than 10, e.g., 1, 2, 3, 4, 5, 6, 7, 8, 9 or 10. In a further embodiment, the number of conservative substitutions in SEQ ID NO: 32 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 33.

In one embodiment, the muramidase comprises or consists of the amino acid sequence of SEQ ID NO: 33 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 33 or an allelic variant thereof and a N-terminal and/or C-terminal His-tag and/or HQ-tag. In another aspect, the polypeptide comprises or consists of amino acids 1 to 213 of SEQ ID NO: 33.

In another embodiment, the muramidase is a variant of SEQ ID NO: 33 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 33 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 33 is not more than 10, e.g., 1, 2, 3, 4, 5, 6, 7, 8, 9 or 10. In another embodiment, the number of substitutions, deletions, and/or insertions in SEQ ID NO: 33 is not more than 10, e.g., 1, 2, 3, 4, 5, 6, 7, 8, 9 or 10. In a further embodiment, the number of substitutions, preferably conservative substitutions, in SEQ ID NO: 33 is not more than 10, e.g., 1, 2, 3, 4, 5, 6, 7, 8, 9 or 10. In a further embodiment, the number of conservative substitutions in SEQ ID NO: 33 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 34.

In one embodiment, the muramidase comprises or consists of the amino acid sequence of SEQ ID NO: 34 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 34 or an allelic variant thereof and a N-terminal and/or C-terminal His-tag and/or HQ-tag. In another aspect, the polypeptide comprises or consists of amino acids 1 to 213 of SEQ ID NO: 34.

In another embodiment, the muramidase is a variant of SEQ ID NO: 34 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 34 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 34 is not more than 10, e.g., 1, 2, 3, 4, 5, 6, 7, 8, 9 or 10. In another embodiment, the number of substitutions, deletions, and/or insertions in SEQ ID NO: 34 is not more than 10, e.g., 1, 2, 3, 4, 5, 6, 7, 8, 9 or 10. In a further embodiment, the number of substitutions, preferably conservative substitutions, in SEQ ID NO: 34 is not more than 10, e.g., 1, 2, 3, 4, 5, 6, 7, 8, 9 or 10. In a further embodiment, the number of conservative substitutions in SEQ ID NO: 34 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 35.

In one embodiment, the muramidase comprises or consists of the amino acid sequence of SEQ ID NO: 35 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 35 or an allelic variant thereof and a N-terminal and/or C-terminal His-tag and/or HQ-tag. In another aspect, the polypeptide comprises or consists of amino acids 1 to 213 of SEQ ID NO: 35.

In another embodiment, the muramidase is a variant of SEQ ID NO: 35 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 35 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 35 is not more than 10, e.g., 1, 2, 3, 4, 5, 6, 7, 8, 9 or 10. In another embodiment, the number of substitutions, deletions, and/or insertions in SEQ ID NO: 35 is not more than 10, e.g., 1, 2, 3, 4, 5, 6, 7, 8, 9 or 10. In a further embodiment, the number of substitutions, preferably conservative substitutions, in SEQ ID NO: 35 is not more than 10, e.g., 1, 2, 3, 4, 5, 6, 7, 8, 9 or 10. In a further embodiment, the number of conservative substitutions in SEQ ID NO: 35 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 36.

In one embodiment, the muramidase comprises or consists of the amino acid sequence of SEQ ID NO: 36 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 36 or an allelic variant thereof and a N-terminal and/or C-terminal His-tag and/or HQ-tag. In another aspect, the polypeptide comprises or consists of amino acids 1 to 213 of SEQ ID NO: 36.

In another embodiment, the muramidase is a variant of SEQ ID NO: 36 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 36 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 36 is not more than 10, e.g., 1, 2, 3, 4, 5, 6, 7, 8, 9 or 10. In another embodiment, the number of substitutions, deletions, and/or insertions in SEQ ID NO: 36 is not more than 10, e.g., 1, 2, 3, 4, 5, 6, 7, 8, 9 or 10. In a further embodiment, the number of substitutions, preferably conservative substitutions, in SEQ ID NO: 36 is not more than 10, e.g., 1, 2, 3, 4, 5, 6, 7, 8, 9 or 10. In a further embodiment, the number of conservative substitutions in SEQ ID NO: 36 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 37.

In one embodiment, the muramidase comprises or consists of the amino acid sequence of SEQ ID NO: 37 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 37 or an allelic variant thereof and a N-terminal and/or C-terminal His-tag and/or HQ-tag. In another aspect, the polypeptide comprises or consists of amino acids 1 to 213 of SEQ ID NO: 37.

In another embodiment, the muramidase is a variant of SEQ ID NO: 37 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 37 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 37 is not more than 10, e.g., 1, 2, 3, 4, 5, 6, 7, 8, 9 or 10. In another embodiment, the number of substitutions, deletions, and/or insertions in SEQ ID NO: 37 is not more than 10, e.g., 1, 2, 3, 4, 5, 6, 7, 8, 9 or 10. In a further embodiment, the number of substitutions, preferably conservative substitutions, in SEQ ID NO: 37 is not more than 10, e.g., 1, 2, 3, 4, 5, 6, 7, 8, 9 or 10. In a further embodiment, the number of conservative substitutions in SEQ ID NO: 37 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 38.

In one embodiment, the muramidase comprises or consists of the amino acid sequence of SEQ ID NO: 38 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 38 or an allelic variant thereof and a N-terminal and/or C-terminal His-tag and/or HQ-tag. In another aspect, the polypeptide comprises or consists of amino acids 1 to 213 of SEQ ID NO: 38.

In another embodiment, the muramidase is a variant of SEQ ID NO: 38 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 38 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 38 is not more than 10, e.g., 1, 2, 3, 4, 5, 6, 7, 8, 9 or 10. In another embodiment, the number of substitutions, deletions, and/or insertions in SEQ ID NO: 38 is not more than 10, e.g., 1, 2, 3, 4, 5, 6, 7, 8, 9 or 10. In a further embodiment, the number of substitutions, preferably conservative substitutions, in SEQ ID NO: 38 is not more than 10, e.g., 1, 2, 3, 4, 5, 6, 7, 8, 9 or 10. In a further embodiment, the number of conservative substitutions in SEQ ID NO: 38 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 39.

In one embodiment, the muramidase comprises or consists of the amino acid sequence of SEQ ID NO: 39 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 39 or an allelic variant thereof and a N-terminal and/or C-terminal His-tag and/or HQ-tag. In another aspect, the polypeptide comprises or consists of amino acids 1 to 213 of SEQ ID NO: 39.

In another embodiment, the muramidase is a variant of SEQ ID NO: 39 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 39 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 39 is not more than 10, e.g., 1, 2, 3, 4, 5, 6, 7, 8, 9 or 10. In another embodiment, the number of substitutions, deletions, and/or insertions in SEQ ID NO: 39 is not more than 10, e.g., 1, 2, 3, 4, 5, 6, 7, 8, 9 or 10. In a further embodiment, the number of substitutions, preferably conservative substitutions, in SEQ ID NO: 39 is not more than 10, e.g., 1, 2, 3, 4, 5, 6, 7, 8, 9 or 10. In a further embodiment, the number of conservative substitutions in SEQ ID NO: 39 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 40.

In one embodiment, the muramidase comprises or consists of the amino acid sequence of SEQ ID NO: 40 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 40 or an allelic variant thereof and a N-terminal and/or C-terminal His-tag and/or HQ-tag. In another aspect, the polypeptide comprises or consists of amino acids 1 to 213 of SEQ ID NO: 40.

In another embodiment, the muramidase is a variant of SEQ ID NO: 40 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 40 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 40 is not more than 10, e.g., 1, 2, 3, 4, 5, 6, 7, 8, 9 or 10. In another embodiment, the number of substitutions, deletions, and/or insertions in SEQ ID NO: 40 is not more than 10, e.g., 1, 2, 3, 4, 5, 6, 7, 8, 9 or 10. In a further embodiment, the number of substitutions, preferably conservative substitutions, in SEQ ID NO: 40 is not more than 10, e.g., 1, 2, 3, 4, 5, 6, 7, 8, 9 or 10. In a further embodiment, the number of conservative substitutions in SEQ ID NO: 40 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41.

In one embodiment, the muramidase comprises or consists of the amino acid sequence of SEQ ID NO: 41 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 41 or an allelic variant thereof and a N-terminal and/or C-terminal His-tag and/or HQ-tag. In another aspect, the polypeptide comprises or consists of amino acids 1 to 213 of SEQ ID NO: 41.

In another embodiment, the muramidase is a variant of SEQ ID NO: 41 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 41 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 41 is not more than 10, e.g., 1, 2, 3, 4, 5, 6, 7, 8, 9 or 10. In another embodiment, the number of substitutions, deletions, and/or insertions in SEQ ID NO: 41 is not more than 10, e.g., 1, 2, 3, 4, 5, 6, 7, 8, 9 or 10. In a further embodiment, the number of substitutions, preferably conservative substitutions, in SEQ ID NO: 41 is not more than 10, e.g., 1, 2, 3, 4, 5, 6, 7, 8, 9 or 10. In a further embodiment, the number of conservative substitutions in SEQ ID NO: 41 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 42.

In one embodiment, the muramidase comprises or consists of the amino acid sequence of SEQ ID NO: 42 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 42 or an allelic variant thereof and a N-terminal and/or C-terminal His-tag and/or HQ-tag. In another aspect, the polypeptide comprises or consists of amino acids 1 to 213 of SEQ ID NO: 42.

In another embodiment, the muramidase is a variant of SEQ ID NO: 42 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 42 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 42 is not more than 10, e.g., 1, 2, 3, 4, 5, 6, 7, 8, 9 or 10. In another embodiment, the number of substitutions, deletions, and/or insertions in SEQ ID NO: 42 is not more than 10, e.g., 1, 2, 3, 4, 5, 6, 7, 8, 9 or 10. In a further embodiment, the number of substitutions, preferably conservative substitutions, in SEQ ID NO: 42 is not more than 10, e.g., 1, 2, 3, 4, 5, 6, 7, 8, 9 or 10. In a further embodiment, the number of conservative substitutions in SEQ ID NO: 42 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 43.

In one embodiment, the muramidase comprises or consists of the amino acid sequence of SEQ ID NO: 43 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 43 or an allelic variant thereof and a N-terminal and/or C-terminal His-tag and/or HQ-tag. In another aspect, the polypeptide comprises or consists of amino acids 1 to 213 of SEQ ID NO: 43.

In another embodiment, the muramidase is a variant of SEQ ID NO: 43 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 43 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 43 is not more than 10, e.g., 1, 2, 3, 4, 5, 6, 7, 8, 9 or 10. In another embodiment, the number of substitutions, deletions, and/or insertions in SEQ ID NO: 43 is not more than 10, e.g., 1, 2, 3, 4, 5, 6, 7, 8, 9 or 10. In a further embodiment, the number of substitutions, preferably conservative substitutions, in SEQ ID NO: 43 is not more than 10, e.g., 1, 2, 3, 4, 5, 6, 7, 8, 9 or 10. In a further embodiment, the number of conservative substitutions in SEQ ID NO: 43 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44.

In one embodiment, the muramidase comprises or consists of the amino acid sequence of SEQ ID NO: 44 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 44 or an allelic variant thereof and a N-terminal and/or C-terminal His-tag and/or HQ-tag. In another aspect, the polypeptide comprises or consists of amino acids 1 to 213 of SEQ ID NO: 44.

In another embodiment, the muramidase is a variant of SEQ ID NO: 44 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 44 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 44 is not more than 10, e.g., 1, 2, 3, 4, 5, 6, 7, 8, 9 or 10. In another embodiment, the number of substitutions, deletions, and/or insertions in SEQ ID NO: 44 is not more than 10, e.g., 1, 2, 3, 4, 5, 6, 7, 8, 9 or 10. In a further embodiment, the number of substitutions, preferably conservative substitutions, in SEQ ID NO: 44 is not more than 10, e.g., 1, 2, 3, 4, 5, 6, 7, 8, 9 or 10. In a further embodiment, the number of conservative substitutions in SEQ ID NO: 44 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45.

In one embodiment, the muramidase comprises or consists of the amino acid sequence of SEQ ID NO: 45 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 45 or an allelic variant thereof and a N-terminal and/or C-terminal His-tag and/or HQ-tag. In another aspect, the polypeptide comprises or consists of amino acids 1 to 213 of SEQ ID NO: 45.

In another embodiment, the muramidase is a variant of SEQ ID NO: 45 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 45 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 45 is not more than 10, e.g., 1, 2, 3, 4, 5, 6, 7, 8, 9 or 10. In another embodiment, the number of substitutions, deletions, and/or insertions in SEQ ID NO: 45 is not more than 10, e.g., 1, 2, 3, 4, 5, 6, 7, 8, 9 or 10. In a further embodiment, the number of substitutions, preferably conservative substitutions, in SEQ ID NO: 45 is not more than 10, e.g., 1, 2, 3, 4, 5, 6, 7, 8, 9 or 10. In a further embodiment, the number of conservative substitutions in SEQ ID NO: 45 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 46.

In one embodiment, the muramidase comprises or consists of the amino acid sequence of SEQ ID NO: 46 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 46 or an allelic variant thereof and a N-terminal and/or C-terminal His-tag and/or HQ-tag. In another aspect, the polypeptide comprises or consists of amino acids 1 to 213 of SEQ ID NO: 46.

In another embodiment, the muramidase is a variant of SEQ ID NO: 46 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 46 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 46 is not more than 10, e.g., 1, 2, 3, 4, 5, 6, 7, 8, 9 or 10. In another embodiment, the number of substitutions, deletions, and/or insertions in SEQ ID NO: 46 is not more than 10, e.g., 1, 2, 3, 4, 5, 6, 7, 8, 9 or 10. In a further embodiment, the number of substitutions, preferably conservative substitutions, in SEQ ID NO: 46 is not more than 10, e.g., 1, 2, 3, 4, 5, 6, 7, 8, 9 or 10. In a further embodiment, the number of conservative substitutions in SEQ ID NO: 46 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47.

In one embodiment, the muramidase comprises or consists of the amino acid sequence of SEQ ID NO: 47 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 47 or an allelic variant thereof and a N-terminal and/or C-terminal His-tag and/or HQ-tag. In another aspect, the polypeptide comprises or consists of amino acids 1 to 213 of SEQ ID NO: 47.

In another embodiment, the muramidase is a variant of SEQ ID NO: 47 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 47 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 47 is not more than 10, e.g., 1, 2, 3, 4, 5, 6, 7, 8, 9 or 10. In another embodiment, the number of substitutions, deletions, and/or insertions in SEQ ID NO: 47 is not more than 10, e.g., 1, 2, 3, 4, 5, 6, 7, 8, 9 or 10. In a further embodiment, the number of substitutions, preferably conservative substitutions, in SEQ ID NO: 47 is not more than 10, e.g., 1, 2, 3, 4, 5, 6, 7, 8, 9 or 10. In a further embodiment, the number of conservative substitutions in SEQ ID NO: 47 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48.

In one embodiment, the muramidase comprises or consists of the amino acid sequence of SEQ ID NO: 48 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 48 or an allelic variant thereof and a N-terminal and/or C-terminal His-tag and/or HQ-tag. In another aspect, the polypeptide comprises or consists of amino acids 1 to 213 of SEQ ID NO: 48.

In another embodiment, the muramidase is a variant of SEQ ID NO: 48 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 48 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 48 is not more than 10, e.g., 1, 2, 3, 4, 5, 6, 7, 8, 9 or 10. In another embodiment, the number of substitutions, deletions, and/or insertions in SEQ ID NO: 48 is not more than 10, e.g., 1, 2, 3, 4, 5, 6, 7, 8, 9 or 10. In a further embodiment, the number of substitutions, preferably conservative substitutions, in SEQ ID NO: 48 is not more than 10, e.g., 1, 2, 3, 4, 5, 6, 7, 8, 9 or 10. In a further embodiment, the number of conservative substitutions in SEQ ID NO: 48 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 49.

In one embodiment, the muramidase comprises or consists of the amino acid sequence of SEQ ID NO: 49 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 49 or an allelic variant thereof and a N-terminal and/or C-terminal His-tag and/or HQ-tag. In another aspect, the polypeptide comprises or consists of amino acids 1 to 213 of SEQ ID NO: 49.

In another embodiment, the muramidase is a variant of SEQ ID NO: 49 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 49 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 49 is not more than 10, e.g., 1, 2, 3, 4, 5, 6, 7, 8, 9 or 10. In another embodiment, the number of substitutions, deletions, and/or insertions in SEQ ID NO: 49 is not more than 10, e.g., 1, 2, 3, 4, 5, 6, 7, 8, 9 or 10. In a further embodiment, the number of substitutions, preferably conservative substitutions, in SEQ ID NO: 49 is not more than 10, e.g., 1, 2, 3, 4, 5, 6, 7, 8, 9 or 10. In a further embodiment, the number of conservative substitutions in SEQ ID NO: 49 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 50.

In one embodiment, the muramidase comprises or consists of the amino acid sequence of SEQ ID NO: 50 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 50 or an allelic variant thereof and a N-terminal and/or C-terminal His-tag and/or HQ-tag. In another aspect, the polypeptide comprises or consists of amino acids 1 to 213 of SEQ ID NO: 50.

In another embodiment, the muramidase is a variant of SEQ ID NO: 50 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 50 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 50 is not more than 10, e.g., 1, 2, 3, 4, 5, 6, 7, 8, 9 or 10. In another embodiment, the number of substitutions, deletions, and/or insertions in SEQ ID NO: 50 is not more than 10, e.g., 1, 2, 3, 4, 5, 6, 7, 8, 9 or 10. In a further embodiment, the number of substitutions, preferably conservative substitutions, in SEQ ID NO: 50 is not more than 10, e.g., 1, 2, 3, 4, 5, 6, 7, 8, 9 or 10. In a further embodiment, the number of conservative substitutions in SEQ ID NO: 50 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 51.

In one embodiment, the muramidase comprises or consists of the amino acid sequence of SEQ ID NO: 51 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 51 or an allelic variant thereof and a N-terminal and/or C-terminal His-tag and/or HQ-tag. In another aspect, the polypeptide comprises or consists of amino acids 1 to 213 of SEQ ID NO: 51.

In another embodiment, the muramidase is a variant of SEQ ID NO: 51 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 51 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 51 is not more than 10, e.g., 1, 2, 3, 4, 5, 6, 7, 8, 9 or 10. In another embodiment, the number of substitutions, deletions, and/or insertions in SEQ ID NO: 51 is not more than 10, e.g., 1, 2, 3, 4, 5, 6, 7, 8, 9 or 10. In a further embodiment, the number of substitutions, preferably conservative substitutions, in SEQ ID NO: 51 is not more than 10, e.g., 1, 2, 3, 4, 5, 6, 7, 8, 9 or 10. In a further embodiment, the number of conservative substitutions in SEQ ID NO: 51 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 52.

In one embodiment, the muramidase comprises or consists of the amino acid sequence of SEQ ID NO: 52 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 52 or an allelic variant thereof and a N-terminal and/or C-terminal His-tag and/or HQ-tag. In another aspect, the polypeptide comprises or consists of amino acids 1 to 213 of SEQ ID NO: 52.

In another embodiment, the muramidase is a variant of SEQ ID NO: 52 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 52 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 52 is not more than 10, e.g., 1, 2, 3, 4, 5, 6, 7, 8, 9 or 10. In another embodiment, the number of substitutions, deletions, and/or insertions in SEQ ID NO: 52 is not more than 10, e.g., 1, 2, 3, 4, 5, 6, 7, 8, 9 or 10. In a further embodiment, the number of substitutions, preferably conservative substitutions, in SEQ ID NO: 52 is not more than 10, e.g., 1, 2, 3, 4, 5, 6, 7, 8, 9 or 10. In a further embodiment, the number of conservative substitutions in SEQ ID NO: 52 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 53.

In one embodiment, the muramidase comprises or consists of the amino acid sequence of SEQ ID NO: 53 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 53 or an allelic variant thereof and a N-terminal and/or C-terminal His-tag and/or HQ-tag. In another aspect, the polypeptide comprises or consists of amino acids 1 to 213 of SEQ ID NO: 53.

In another embodiment, the muramidase is a variant of SEQ ID NO: 53 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 53 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 53 is not more than 10, e.g., 1, 2, 3, 4, 5, 6, 7, 8, 9 or 10. In another embodiment, the number of substitutions, deletions, and/or insertions in SEQ ID NO: 53 is not more than 10, e.g., 1, 2, 3, 4, 5, 6, 7, 8, 9 or 10. In a further embodiment, the number of substitutions, preferably conservative substitutions, in SEQ ID NO: 53 is not more than 10, e.g., 1, 2, 3, 4, 5, 6, 7, 8, 9 or 10. In a further embodiment, the number of conservative substitutions in SEQ ID NO: 53 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 54.

In one embodiment, the muramidase comprises or consists of the amino acid sequence of SEQ ID NO: 54 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 54 or an allelic variant thereof and a N-terminal and/or C-terminal His-tag and/or HQ-tag. In another aspect, the polypeptide comprises or consists of amino acids 1 to 213 of SEQ ID NO: 54.

In another embodiment, the muramidase is a variant of SEQ ID NO: 54 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 54 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 54 is not more than 10, e.g., 1, 2, 3, 4, 5, 6, 7, 8, 9 or 10. In another embodiment, the number of substitutions, deletions, and/or insertions in SEQ ID NO: 54 is not more than 10, e.g., 1, 2, 3, 4, 5, 6, 7, 8, 9 or 10. In a further embodiment, the number of substitutions, preferably conservative substitutions, in SEQ ID NO: 54 is not more than 10, e.g., 1, 2, 3, 4, 5, 6, 7, 8, 9 or 10. In a further embodiment, the number of conservative substitutions in SEQ ID NO: 54 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 55.

In one embodiment, the muramidase comprises or consists of the amino acid sequence of SEQ ID NO: 55 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 55 or an allelic variant thereof and a N-terminal and/or C-terminal His-tag and/or HQ-tag. In another aspect, the polypeptide comprises or consists of amino acids 1 to 213 of SEQ ID NO: 55.

In another embodiment, the muramidase is a variant of SEQ ID NO: 55 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 55 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 55 is not more than 10, e.g., 1, 2, 3, 4, 5, 6, 7, 8, 9 or 10. In another embodiment, the number of substitutions, deletions, and/or insertions in SEQ ID NO: 55 is not more than 10, e.g., 1, 2, 3, 4, 5, 6, 7, 8, 9 or 10. In a further embodiment, the number of substitutions, preferably conservative substitutions, in SEQ ID NO: 55 is not more than 10, e.g., 1, 2, 3, 4, 5, 6, 7, 8, 9 or 10. In a further embodiment, the number of conservative substitutions in SEQ ID NO: 55 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56.

In one embodiment, the muramidase comprises or consists of the amino acid sequence of SEQ ID NO: 56 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 56 or an allelic variant thereof and a N-terminal and/or C-terminal His-tag and/or HQ-tag. In another aspect, the polypeptide comprises or consists of amino acids 1 to 213 of SEQ ID NO: 56.

In another embodiment, the muramidase is a variant of SEQ ID NO: 56 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 56 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 56 is not more than 10, e.g., 1, 2, 3, 4, 5, 6, 7, 8, 9 or 10. In another embodiment, the number of substitutions, deletions, and/or insertions in SEQ ID NO: 56 is not more than 10, e.g., 1, 2, 3, 4, 5, 6, 7, 8, 9 or 10. In a further embodiment, the number of substitutions, preferably conservative substitutions, in SEQ ID NO: 56 is not more than 10, e.g., 1, 2, 3, 4, 5, 6, 7, 8, 9 or 10. In a further embodiment, the number of conservative substitutions in SEQ ID NO: 56 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 57.

In one embodiment, the muramidase comprises or consists of the amino acid sequence of SEQ ID NO: 57 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 57 or an allelic variant thereof and a N-terminal and/or C-terminal His-tag and/or HQ-tag. In another aspect, the polypeptide comprises or consists of amino acids 1 to 213 of SEQ ID NO: 57.

In another embodiment, the muramidase is a variant of SEQ ID NO: 57 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 57 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 57 is not more than 10, e.g., 1, 2, 3, 4, 5, 6, 7, 8, 9 or 10. In another embodiment, the number of substitutions, deletions, and/or insertions in SEQ ID NO: 57 is not more than 10, e.g., 1, 2, 3, 4, 5, 6, 7, 8, 9 or 10. In a further embodiment, the number of substitutions, preferably conservative substitutions, in SEQ ID NO: 57 is not more than 10, e.g., 1, 2, 3, 4, 5, 6, 7, 8, 9 or 10. In a further embodiment, the number of conservative substitutions in SEQ ID NO: 57 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 58.

In one embodiment, the muramidase comprises or consists of the amino acid sequence of SEQ ID NO: 58 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 58 or an allelic variant thereof and a N-terminal and/or C-terminal His-tag and/or HQ-tag. In another aspect, the polypeptide comprises or consists of amino acids 1 to 213 of SEQ ID NO: 58.

In another embodiment, the muramidase is a variant of SEQ ID NO: 58 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 58 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 58 is not more than 10, e.g., 1, 2, 3, 4, 5, 6, 7, 8, 9 or 10. In another embodiment, the number of substitutions, deletions, and/or insertions in SEQ ID NO: 58 is not more than 10, e.g., 1, 2, 3, 4, 5, 6, 7, 8, 9 or 10. In a further embodiment, the number of substitutions, preferably conservative substitutions, in SEQ ID NO: 58 is not more than 10, e.g., 1, 2, 3, 4, 5, 6, 7, 8, 9 or 10. In a further embodiment, the number of conservative substitutions in SEQ ID NO: 58 is not more than 10, e.g., 1, 2, 3, 4, 5, 6, 7, 8, 9 or 10.

In one embodiment, the muramidase has at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59.

In one embodiment, the muramidase comprises or consists of the amino acid sequence of SEQ ID NO: 59 or an allelic variant thereof; or is a fragment thereof having muramidase activity, wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids. In another embodiment, the muramidase comprises or consists of the amino acid sequence of SEQ ID NO: 59 or an allelic variant thereof and a N-terminal and/or C-terminal His-tag and/or HQ-tag. In another aspect, the polypeptide comprises or consists of amino acids 1 to 213 of SEQ ID NO: 59.

In another embodiment, the muramidase is a variant of SEQ ID NO: 59 wherein the variant has muramidase activity and comprises one or more substitutions, and/or one or more deletions, and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions. In another embodiment, the number of positions comprising one or more amino acid substitutions, and/or one or more amino acid deletions, and/or one or more amino acid insertions or any combination thereof in SEQ ID NO: 59 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 59 is not more than 10, e.g., 1, 2, 3, 4, 5, 6, 7, 8, 9 or 10. In another embodiment, the number of substitutions, deletions, and/or insertions in SEQ ID NO: 59 is not more than 10, e.g., 1, 2, 3, 4, 5, 6, 7, 8, 9 or 10. In a further embodiment, the number of substitutions, preferably conservative substitutions, in SEQ ID NO: 59 is not more than 10, e.g., 1, 2, 3, 4, 5, 6, 7, 8, 9 or 10. In a further embodiment, the number of conservative substitutions in SEQ

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an aminoterminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, AlaNal, Ser/Gly, Tyr/Phe, Ala/Pro, Lys/Arg, Asp/Asn, Leu/Ile, LeuNal, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The crystal structure of the Acremonium alcalophilum CBS114.92 muramidase was solved at a resolution of 1.3 Å as disclosed in WO 2013/076253. These abullic coordinates can be used to generate a three dimensional model depicting the structure of the Acremonium alcalophilum CBS114.92 muramidase or homologous structures (such as the variants of the present invention). Using the x/ray structure, amino acid residues D95 and E97 were identified as catalytic residues.

In a preferred embodiment, the invention relates to a method of improving the Dry Matter digestibility (DMd) and/or volatile fatty acid (VFA) production and/or meat production and/or milk production of a ruminant comprising administering a ruminant feed, ruminant feed supplement or ruminant feed additive comprising one or more muramidases to the ruminant, wherein:

(a) the muramidase is obtained or obtainable from the phylum Ascomycota, and is dosed at a level of 1 to 200 mg enzyme protein per kg ruminant feed Dry Matter (DM);
(b) the ruminant is a selected from the group consisting of: cattle, cow, dairy cattle, beef cattle, buffalo, calf, goat, sheep, lamb, deer, yak, camel and llama;
(c) the Dry Matter digestibility (DMd) and/or volatile fatty acid (VFA) production and/or meat production and/or milk production is improved by at least 1% compared to control; and
(d) optionally the muramidase is provided to the ruminant on a daily basis for at least 30 days during the life span of the ruminant.

In one embodiment, the method is provided to growing ruminants. In one embodiment, the method is provided to dairy cattle. In a further embodiment, the method is provided to dairy cattle during lactation. In one embodiment, the method is provided to beef cattle in the growing phase of beef cattle production. In one embodiment, the method is provided to beef cattle in the finishing phase of beef cattle production.

In one embodiment, the energy corrected milk (ECM) production is improved by at least 1.25%, such as by at least 1.5%, at least 1.75% or at least 2.0% compared to the control. In another embodiment, the ECM production is improved by between 1% and 5%, such as between 1% and 4%, between 1% and 3%, between 1.25% and 2.5%, or between 1.5% and 2% compared to the control, or any combination of these intervals.

In one embodiment, the ruminal dry matter digestibility (DMd) is improved by at least 1%, such as by at least 1.5%, at least 1.75% or at least 2.0% compared to the control. In another embodiment, the dry matter digestibility is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is selected from acetate, propionate, butyrate, isobutyrate, valerate, isovalerate and any combination thereof. In a further embodiment, the volatile fatty acid (VFA) is selected from acetate, propionate, butyrate and any combination thereof. In a yet further embodiment, the volatile fatty acid (VFA) is acetate and/or propionate.

In one embodiment of the method, the volatile fatty acid (VFA) is improved by at least 1%, such as by at least 1.5%, at least 1.75% or at least 2.0% compared to the control. In another embodiment, the volatile fatty acid is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is acetate. In a further embodiment, acetate is improved by at least 1%, such as by at least 1.5%, at least 1.75% or at least 2.0% compared to the control. In another embodiment, acetate is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is propionate. In a further embodiment, propionate is improved by at least 1%, such as by at least 1.5%, at least 1.75% or at least 2.0% compared to the control. In another embodiment, propionate is improved by between 1% and 15%, such as between 1% and 10%, between 1% and 7%, 1% and 5%, or 2% and 5% compared to the control, or any combination of these intervals.

In one embodiment of the method, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed DM, or any combination of these intervals.

In one embodiment, the muramidase is provided to the ruminant using one of the regimes as disclosed herein.

In another preferred embodiment, the invention relates to a method of improving the Dry Matter digestibility (DMd) and/or volatile fatty acid (VFA) production and/or meat production and/or milk production of a ruminant comprising administering a ruminant feed, ruminant feed supplement or ruminant feed additive comprising one or more muramidases to the ruminant, wherein:

(a) the muramidase is a GH24 muramidase obtained or obtainable from the phylum Ascomycota, is dosed at a level of 1 to 200 mg enzyme protein per kg ruminant feed DM;
(b) the ruminant is a selected from the group consisting of: cattle, cow, dairy cattle, beef cattle, buffalo, calf, goat, sheep, lamb, deer, yak, camel and llama;
(c) the Dry Matter digestibility (DMd) and/or volatile fatty acid (VFA) production and/or meat production and/or milk production is improved by at least 1% compared to control; and
(d) optionally the muramidase is provided to the ruminant on a daily basis for at least 30 days during the life span of the ruminant.

In one embodiment, the method is provided to growing ruminants. In one embodiment, the method is provided to dairy cattle. In a further embodiment, the method is provided to dairy cattle during lactation. In one embodiment, the method is provided to beef cattle in the growing phase of beef cattle production. In one embodiment, the method is provided to beef cattle in the finishing phase of beef cattle production.

In one embodiment of the method, the energy corrected milk (ECM) production is improved by at least 1.25%, such as by at least 1.5%, at least 1.75% or at least 2.0% compared to the control. In another embodiment, the ECM production is improved by between 1% and 5%, such as between 1% and 4%, between 1% and 3%, between 1.25% and 2.5%, or between 1.5% and 2% compared to the control, or any combination of these intervals.

In one embodiment of the method, the ruminal dry matter digestibility (DMd) is improved by at least 1%, such as by at least 1.5%, at least 1.75% or at least 2.0% compared to the control. In another embodiment, the dry matter digestibility is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is selected from acetate, propionate, butyrate, isobutyrate, valerate, isovalerate and any combination thereof. In a further embodiment, the volatile fatty acid (VFA) is selected from acetate, propionate, butyrate and any combination thereof. In a yet further embodiment, the volatile fatty acid (VFA) is acetate and/or propionate.

In one embodiment of the method, the volatile fatty acid (VFA) is improved by at least 1%, such as by at least 1.5%, at least 1.75% or at least 2.0% compared to the control. In another embodiment, the volatile fatty acid is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is acetate. In a further embodiment, acetate is improved by at least 1%, such as by at least 1.5%, at least 1.75% or at least 2.0% compared to the control. In another embodiment, acetate is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is propionate. In a further embodiment, propionate is improved by at least 1%, such as by at least 1.5%, at least 1.75% or at least 2.0% compared to the control. In another embodiment, propionate is improved by between 1% and 15%, such as between 1% and 10%, between 1% and 7%, 1% and 5%, or 2% and 5% compared to the control, or any combination of these intervals.

In one embodiment of the method, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed DM, or any combination of these intervals.

In one embodiment of the method, the muramidase is provided to the ruminant using one of the regimes as disclosed herein.

In another preferred embodiment, the invention relates to a method of improving the Dry Matter digestibility (DMd) and/or volatile fatty acid (VFA) production and/or meat production and/or milk production of a ruminant comprising administering a ruminant feed, ruminant feed supplement or ruminant feed additive comprising one or more muramidases to the ruminant, wherein:

(a) the muramidase is a GH25 muramidase obtained or obtainable from the phylum Ascomycota, is dosed at a level of 1 to 200 mg enzyme protein per kg ruminant feed DM;
(b) the ruminant is a selected from the group consisting of: cattle, cow, dairy cattle, beef cattle, buffalo, calf, goat, sheep, lamb, deer, yak, camel and llama;
(c) the Dry Matter digestibility (DMd) and/or volatile fatty acid (VFA) production and/or meat production and/or milk production is improved by at least 1% compared to control; and
(d) optionally the muramidase is provided to the ruminant on a daily basis for at least 30 days during the life span of the ruminant.

In one embodiment, the method is provided to growing ruminants. In one embodiment, the method is provided to dairy cattle. In a further embodiment, the method is provided to dairy cattle during lactation. In one embodiment, the method is provided to beef cattle in the growing phase of beef cattle production. In one embodiment, the method is provided to beef cattle in the finishing phase of beef cattle production.

In one embodiment of the method, the energy corrected milk (ECM) production is improved by at least 1.25%, such as by at least 1.5%, at least 1.75% or at least 2.0% compared to the control. In another embodiment, the ECM production is improved by between 1% and 5%, such as between 1% and 4%, between 1% and 3%, between 1.25% and 2.5%, or between 1.5% and 2% compared to the control, or any combination of these intervals.

In one embodiment of the method, the ruminal dry matter digestibility (DMd) is improved by at least 1%, such as by at least 1.5%, at least 1.75% or at least 2.0% compared to the control. In another embodiment, the dry matter digestibility is improved by between 1% and 15%, such as between 1% and 10%, between 1% and 7%, 1% and 5%, or 2% and 5% compared to the control, or any combination of these intervals.

In one embodiment of the method, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed, or any combination of these intervals.

In one embodiment of the method, the volatile fatty acid (VFA) is selected from acetate, propionate, butyrate, isobutyrate, valerate, isovalerate and any combination thereof. In a further embodiment, the volatile fatty acid (VFA) is selected from acetate, propionate, butyrate and any combination thereof. In a yet further embodiment, the volatile fatty acid (VFA) is acetate and/or propionate.

In one embodiment of the method, the volatile fatty acid (VFA) is improved by at least 1%, such as by at least 1.5%, at least 1.75% or at least 2.0% compared to the control. In another embodiment, the volatile fatty acid is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is acetate. In a further embodiment, acetate is improved by at least 1%, such as by at least 1.5%, at least 1.75% or at least 2.0% compared to the control. In another embodiment, acetate is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is propionate. In a further embodinvent, propionate is improved by at least 1%, such as by at least 1.5%, at least 1.75% or at least 2.0% compared to the control. In another embodiment, propionate is improved by between 1% and 15%, such as between 1% and 10%, between 1% and 7%, 1% and 5%, or 2% and 5% compared to the control, or any combination of these intervals.

In one embodiment of the method, the muramidase is provided to the ruminant using one of the regimes as disclosed herein.

In another preferred embodiment, the invention relates to a method of improving the Dry Matter digestibility (DMd) and/or volatile fatty acid (VFA) production and/or meat production and/or milk production of a ruminant comprising administering a ruminant feed, ruminant feed supplement or ruminant feed additive comprising one or more muramidases to the ruminant, wherein:

(a) the muramidase is a novel MUR polypeptide having muramidase activity muramidase obtained or obtainable from the phylum Ascomycota, is dosed at a level of 1 to 200 mg enzyme protein per kg ruminant feed DM;
(b) the ruminant is a selected from the group consisting of: cattle, cow, dairy cattle, beef cattle, buffalo, calf, goat, sheep, lamb, deer, yak, camel and llama;
(c) the Dry Matter digestibility (DMd) and/or volatile fatty acid (VFA) production and/or meat production and/or milk production is improved by at least 1% compared to control; and
(d) optionally the muramidase is provided to the ruminant on a daily basis for at least 30 days during the life span of the ruminant.

In one embodiment, the method is provided to growing ruminants. In one embodiment, the method is provided to dairy cattle. In a further embodiment, the method is provided to dairy cattle during lactation. In one embodiment, the method is provided to beef cattle in the growing phase of beef cattle production. In one embodiment, the method is provided to beef cattle in the finishing phase of beef cattle production.

In one embodiment of the method, the energy corrected milk (ECM) production is improved by at least 1.25%, such as by at least 1.5%, at least 1.75% or at least 2.0% compared to the control. In another embodiment, the ECM production is improved by between 1% and 5%, such as between 1% and 4%, between 1% and 3%, between 1.25% and 2.5%, or between 1.5% and 2% compared to the control, or any combination of these intervals.

In one embodiment of the method, the ruminal dry matter digestibility (DMd) is improved by at least 1%, such as by at least 1.5%, at least 1.75% or at least 2.0% compared to the control. In another embodiment, the dry matter digestibility is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is selected from acetate, propionate, butyrate, isobutyrate, valerate, isovalerate and any combination thereof. In a further embodiment, the volatile fatty acid (VFA) is selected from acetate, propionate, butyrate and any combination thereof. In a yet further embodiment, the volatile fatty acid (VFA) is acetate and/or propionate.

In one embodiment of the method, the volatile fatty acid (VFA) is improved by at least 1%, such as by at least 1.5%, at least 1.75% or at least 2.0% compared to the control. In another embodiment, the volatile fatty acid is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is acetate. In a further embodiment, acetate is improved by at least 1%, such as by at least 1.5%, at least 1.75% or at least 2.0% compared to the control. In another embodiment, acetate is improved by between 1% and 15%, such as between 1% and 10%, between 1% and 7%, 1% and 5%, or 2% and 5% compared to the control, or any combination of these intervals.

In one embodiment of the method, the volatile fatty acid (VFA) is propionate. In a further embodinvent, propionate is improved by at least 1%, such as by at least 1.5%, at least 1.75% or at least 2.0% compared to the control. In another embodiment, propionate is improved by between 1% and 15%, such as between 1% and 10%, between 1% and 7%, 1% and 5%, or 2% and 5% compared to the control, or any combination of these intervals.

In one embodiment of the method, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed DM, or any combination of these intervals.

In one embodiment of the method, the muramidase is provided to the ruminant using one of the regimes as disclosed herein.

### Formulating Agent

The enzyme of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the ruminants.

For a solid formulation, the formulation may be for example as a granule, spray dried powder or agglomerate. The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In an embodiment, the solid composition is in granulated form. The granule may have a matrix structure where the components are mixed homogeneously. However, the granule typically comprises a core particle and one or more coatings, which typically are salt and/or wax coatings. Examples of waxes are polyethylene glycols; polypropylenes; Carnauba wax; Candelilla wax; bees wax; hydrogenated plant oil or ruminant tallow such as hydrogenated ox tallow, hydrogenated palm oil, hydrogenated cotton seeds and/or hydrogenated soy bean oil; fatty acid alcohols;

mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil. The core particle can either be a homogeneous blend of muramidase of the invention optionally combined with one or more additional enzymes and optionally together with one or more salts or an inert particle with the muramidase of the invention optionally combined with one or more additional enzymes applied onto it.

In an embodiment, the material of the core particles are selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In a preferred embodiment, the core comprises a clay mineral such as kaolinite or kaolin.

The salt coating is typically at least 1 μm thick and can either be one particular salt or a mixture of salts, such as Na₂SO₄, K₂SO₄, MgSO₄ and/or sodium citrate. Other examples are those described in e.g. WO 2008/017659, WO 2006/034710, WO 1997/05245, WO 1998/54980, WO 1998/55599, WO 2000/70034 or polymer coating such as described in WO 2001/00042.

In another embodiment, the composition is a solid composition comprising the muramidase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate and calcium carbonate. In a preferred embodiment, the solid composition is in granulated form. In an embodiment, the solid composition is in granulated form and comprises a core particle, an enzyme layer comprising the muramidase of the invention and a salt coating.

In a further embodiment, the formulating agent is selected from one or more of the following compounds: glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose. In a preferred embodiment, the formulating agent is selected from one or more of the following compounds: 1, 2-propylene glycol, 1, 3-propylene glycol, sodium sulfate, dextrin, cellulose, sodium thiosulfate, kaolin and calcium carbonate.

Ruminant Feed, Ruminant Feed Supplement and Ruminant Feed Additives A ruminant feed composition or component according to the invention has a crude protein content of between 50 and 800 g/kg, and furthermore comprises one or more polypeptides having muramidase activity as described herein.

Furthermore, or in the alternative (to the crude protein content indicated above), the ruminant feed composition of the invention has a content of metabolisable energy of 5-30 MJ/kg.

In particular embodiments, the content of metabolisable energy, crude protein, calcium and/or phosphorus is within any one of ranges 2, 3, 4 or 5 in Table B of WO 2001/058275 (R. 2-5).

In particular embodiments, the ruminant feed comprises non-protein nitrogen obtained from e.g. urea.

The nitrogen content is determined by the Kjeldahl method (A.O.A.C., 1984, Official Methods of Analysis 14th ed., Association of Official Analytical Chemists, Washington D.C.) and crude protein is calculated as nitrogen (N) multiplied by a factor 6.25 (i.e. Crude protein (g/kg)=N (g/kg)×6.25).

Metabolisable energy can be calculated on the basis of the NRC publication Nutrient requirements in ruminant, seventh revised edition 2001, subcommittee on ruminant nutrition, committee on ruminant nutrition, board of agriculture, national research council. National Academy Press, Washington, D.C., pp. 2-6.

In a particular embodiment, the ruminant feed composition of the invention contains at least one vegetable protein as defined above.

The ruminant feed composition of the invention may also comprise Dried Distillers Grains with Solubles (DDGS), typically in amounts of 0-30%.

In still further particular embodiments, the ruminant feed composition of the invention contains 0-80% maize; and/or 0-80% sorghum; and/or 0-70% wheat; and/or 0-70% Barley; and/or 0-30% oats; and/or 0-40% soybean meal; and/or 0-20% whey.

The ruminant feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g.

beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

In a particular embodiment forage plants such as corn (maize), legumes, and grasses that have been chopped and anaerobically stored and fermented for preservation. This is known as silage or ensilage and can compile up to 90% of cattle diets.

In a particular embodiment non-protein nitrogen (NPN) sources can make part of the diet. An example is urea making up to 25% of the total Crude Protein of cattle diets.

Ruminant concentrate comprising many feedstuffs can e.g. be manufactured as mash feed (nonpelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) muramidase/enzyme preparation may also be added before or during the feed ingredient step. Typically, a liquid enzyme preparation comprises the muramidase of the invention optionally with a polyol, such as glycerol, ethylene glycol or propylene glycol, and is added after the pelleting step, such as by spraying the liquid formulation onto the pellets. The muramidase may also be incorporated in a feed supplement, a feed additive or a premix.

Alternatively, the muramidase can be prepared by freezing a mixture of liquid enzyme solution with a bulking agent such as ground soybean meal, and then lyophilizing the mixture.

In an embodiment, the composition comprises one or more additional enzymes. In an embodiment, the composition comprises one or more microbes. In an embodiment, the composition comprises one or more vitamins. In an embodiment, the composition comprises one or more minerals.

In an embodiment, the composition comprises one or more amino acids. In an embodiment, the composition comprises one or more other feed ingredients.

In another embodiment, the composition comprises one or more of the polypeptides of the invention, one or more formulating agents and one or more additional enzymes. In an embodiment, the composition comprises one or more of the polypeptides of the invention, one or more formulating agents and one or more microbes. In an embodiment, the composition comprises one or more of the polypeptides of the invention, one or more formulating agents and one or more vitamins. In an embodiment, the composition comprises one or more of the polypeptides of the invention and one or more minerals. In an embodiment, the composition comprises the polypeptide of the invention, one or more formulating agents and one or more amino acids. In an embodiment, the composition comprises one or more of the polypeptides of the invention, one or more formulating agents and one or more other feed ingredients.

In a further embodiment, the composition comprises one or more of the polypeptides of the invention, one or more formulating agents and one or more components selected from the list consisting of: one or more additional enzymes; one or more microbes; one or more vitamins; one or more minerals; one or more amino acids; and one or more other feed ingredients.

The final muramidase concentration in the diet is within the range of 0.01 to 200 mg enzyme protein per kg ruminant feed DM, such as 0.1 to 150 mg, 0.5 to 100 mg, 1 to 75 mg, 2 to 50 mg, 3 to 25 mg, 2 to 80 mg, 5 to 60 mg, 8 to 40 mg or 10 to 30 mg enzyme protein per kg ruminant feed DM, or any combination of these intervals.

It is at present contemplated that the muramidase is administered in one or more of the following amounts (dosage ranges): 0.01-200; 0.01-100; 0.5-100; 1-50; 5-100; 5-50; 10-100; 0.05-50; 5-25; or 0.10-10—all these ranges being in mg muramidase per kg feed DM (ppm).

For determining mg muramidase protein per kg feed DM, the muramidase is purified from the feed composition, and the specific activity of the purified muramidase is determined using a relevant assay (see under muramidase activity). The muramidase activity of the feed composition as such is also determined using the same assay, and on the basis of these two determinations, the dosage in mg muramidase protein per kg feed is calculated.

In a particular embodiment, the ruminant feed additive of the invention is intended for being included (or prescribed as having to be included) in ruminant diets or feed at levels of 0.01 to 10.0%; more particularly 0.05 to 5.0%; or 0.2 to 1.0% (% meaning g additive per 100 g feed). This is so in particular for premixes.

The same principles apply for determining mg muramidase protein in feed supplement and feed additives. Of course, if a sample is available of the muramidase used for preparing the feed additive or the feed, the specific activity is determined from this sample (no need to purify the muramidase from the feed composition, feed supplement or the feed additive).

### Additional Enzymes

In another embodiment, the compositions described herein optionally include one or more enzymes. Enzymes can be classified on the basis of the handbook Enzyme Nomenclature from NCIUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, muramidase and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition of the invention may also comprise at least one other enzyme selected from the group comprising of xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any combination thereof.

In a particular embodiment, the composition of the invention comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Finase® and Quantum® Blue (AB Enzymes), OptiPhos® (Huvepharma) Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention comprises a xylanase (EC 3.2.1.8). Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (Xylanase/beta-glucanase, DuPont).

In a particular embodiment, the composition of the invention comprises a protease (EC 3.4). Examples of commercially available proteases include Ronozyme® ProAct (DSM Nutritional Products).

In a particular embodiment, the composition of the invention comprises an alpha amylase (EC 3.2.1.1). Examples of commercially available alpha-amylases include Ronozyme® Rumistar (DSM Nutritional Products).

### Microbes

In an embodiment, the ruminant feed composition further comprises one or more additional microbes. In a particular embodiment, the ruminant feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, ruminant feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, the composition, ruminant feed supplement, ruminant feed additive or ruminant feed further comprises a bacterium selected from one or more of the following strains: Enterococcus faecium strain 8G-1 (NRRL B-50173), Enterococcus faecium strain 8G-73 (NRRL B-50172), Bacillus pumilus strain 8G-134 (NRRL B-50174), M. elsdenii strain NCIMB 41125, Propionibacterium strain P169 (PTA-5271), Propionibacterium strain P170 (PTA-5272) strains, Propionibacterium strain P179 (NRRL B-50133), Propionibacterium strain P195 (NRRL B-50132), Propionibacterium strain P261 (NRRL B-50131), Propionibacteria jensenii strain P63 (DSM22192), Propionibacterium strain P5 (ATCC 55467), Propionibacterium strain P54 (NRRL B-50494), Propionibacterium strain P25 (NRRL B-50497), Propionibacterium strain P49 (NRRL B-50496), Propionibacterium strain P104 (NRRL B-50495), B. licheniformis strain 3-12a (NRRL B-50504), B. subtilis strain 4-7d (NRRL B-50505), B. licheniformis strain 4-2a (NRRL B-50506), B. subtilis strain 3-5h (NRRL B-50507), Bacillus 747 (NRRL B-67257) or a strain having all of the identifying characteristics of Bacillus 747 (NRRL B-67257), Bacillus strain 1104 (NRRL B-67258), Bacillus strain 1781 (NRRL B-67259), Bacillus strain 1541 (NRRL B-67260), Bacillus strain 2018 (NRRL B-67261), and Bacillus strain 1999 (NRRL B-67318).

In a more preferred embodiment, the composition, ruminant feed supplement, ruminant feed additive or ruminant feed further comprises a bacterium from one or more of the following strains of Bacillus subtilis: 3A-P4 (PTA-6506), 15A-P4 (PTA-6507), 22C-P1 (PTA-6508), 2084 (NRRL B500130), LSSA01 (NRRL-B-50104), BS27 (NRRL B-501 05), BS 18 (NRRL B-50633), BS 278 (NRRL B-50634), DSM 29870, DSM 29871, NRRL B-50136, NRRL B-50605, NRRL B-50606, NRRL B-50622 and PTA-7547.

In a more preferred embodiment, the composition, ruminant feed supplement, ruminant feed additive or ruminant feed further comprises a bacterium from one or more of the following strains of Bacillus pumilus: NRRL B-50016, ATCC 700385, NRRL B-50885 or NRRL B-50886.

In a more preferred embodiment, the composition, ruminant feed supplement, ruminant feed additive or ruminant feed further comprises a bacterium from one or more of the following strains of Bacillus lichenformis: NRRL B 50015, NRRL B-50621 or NRRL B-50623.

In a more preferred embodiment, the composition, ruminant feed supplement, ruminant feed additive or ruminant feed further comprises a bacterium from one or more of the following strains of Bacillus amyloliquefaciens: DSM 29869, DSM 29872, NRRL B 50607, PTA-7543, PTA-7549, NRRL B-50349, NRRL B-50606, NRRL B-50013, NRRL B-50151, NRRL B-50141, NRRL B50147 or NRRL B-50888.

The bacterial count of each of the bacterial strains in the ruminant feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the ruminant feed composition is between 1×10⁸ and 1×10¹⁰ CFU/kg of dry matter.

The bacterial count of each of the bacterial strains in the ruminant feed composition is between 1×10⁵ and 1×10¹⁵ CFU/ruminant/day, preferably between 1×10⁷ and 1×10¹³ CFU/ruminant/day, and more preferably between 1×10⁸ and 1×10¹² CFU/ruminant/day. In a more preferred embodiment the bacterial count of each of the bacterial strains in the ruminant feed composition is between 1×10⁹ and 1×10¹¹ CFU/ruminant/day.

In another embodiment, the one or more bacterial strains are present in the form of a stable spore.

### Premix

In an embodiment, the ruminant feed may include a premix, comprising e.g. vitamins, minerals, enzymes, amino acids, preservatives, antibiotics, other feed ingredients or any combination thereof which are mixed into the ruminant feed.

### Amino Acids

The composition of the invention may further comprise one or more amino acids. Examples of

amino acids which are used in ruminant feed are lysine, alanine, beta-alanine, threonine, methionine and tryptophan.

### Vitamins and Minerals

In another embodiment, the ruminant feed may include one or more vitamins, such as one or more fat-soluble vitamins and/or one or more water-soluble vitamins. In another embodiment, the ruminant feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

In the alternative, the ruminant feed supplement or ruminant feed additive of the invention comprises at least one of the individual components specified in “Nutrient requirements in ruminant”, seventh revised edition 2001, subcommittee on ruminant nutrition, committee on ruminant nutrition, board of agriculture, national research council. National Academy Press, Washington, D.C. Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

### Other Feed Ingredients

The composition of the invention may further comprise natural or synthetic colouring agents, gut flora stabilisers, pH stabilisers/pH modulators, digestibility enhancers, growth improving additives, aroma compounds/flavourings, polyunsaturated fatty acids (PUFAs); essential oils, reactive oxygen generating species, anti-fungal peptides, anti-fungal polypeptides, antimicrobial peptides, fungal fermentation extracts and cultures, Immunomodulating additives, anti-oxidative additives, metabolic enhancers, rumen fermentation modifiers, electron receptors and rumen catalysts, other zoo/technological additives, such as binders, anti-caking agents and coagulants, ammonia control agents, botanical antimicrobials, anti-methanogens, and/or ionophores.

Examples of colouring agents include, but are not limited to, carotenoids such as beta-carotene, astaxanthin and lutein.

Examples of gut flora stabilizers and/or pH stabilisers include, but are not limited to, live yeast or yeast cultures such as Saccharomyces cerevisiae.

Examples of digestibility enhancers include, but is not limited to, enzymes e.g. alpha-amylase.

Examples of aroma compounds/flavourings include, but are not limited to, creosol, anethol, deca, undeca- and/or dodeca-lactones, ionones, irone, gingerol, piperidine, propylidene phatalide, butylidene phatalide, capsaicin or tannin.

Examples of polyunsaturated fatty acids include, but are not limited to, C18, C20 and C22 polyunsaturated fatty acids, such as arachidonic acid, docosohexaenoic acid, eicosapentaenoic acid and gamma-linoleic acid.

Examples of essential oils include, but are not limited to, anise, cade, capsicum, cinnamon, clove, dill, garlic, eugenol, or cinnamaldehyde and their active ingredients

Examples of reactive oxygen generating species include, but are not limited to, chemicals such as perborate, persulphate, or percarbonate; and enzymes such as an oxidase, an oxygenase or a syntethase. achidonic acid, docosohexaenoic acid, eicosapentaenoic acid and gamma-linoleic acid.

Examples of antifungal polypeptides (AFP's) include, but are not limited to, the Aspergillus giganteus, and Aspergillus niger peptides, as well as variants and fragments thereof which retain anti-fungal activity, as disclosed in WO 94/01459 and WO 02/090384.

Examples of stabilizing agents such as e.g. buffers and/or acidifiers include, but are not limited to, Live yeast, Sodium Bicarbonate, Calcareous Marine algae and Lecithins

Examples of antimicrobial peptides (AMP's) include, but are not limited to, CAP18, Leucocin A, Tritrpticin, Protegrin-1, Thanatin, Defensin, Lactoferrin, Lactoferricin, and Ovispirin such as Novispirin (Robert Lehrer, 2000), Plectasins, Statins, including the compounds and polypeptides disclosed in WO 03/044049 and WO 03/048148, as well as variants or fragments of the above that retain antimicrobial activity.

Examples of Immunomodulating agents include, but are not limited to, B-glucans, Saccharomyces cerevisiae.

Example of anti-oxidative agents include, but are not limited to, vitamins A, E, and other natural antioxidants; e.g., lecithin.

Examples of electron receptor agents include, but are not limited to, nitrate and its organic compounds.

Examples of fungal fermentation extracts and cultures include, but are not limited to, Aspergillus oryzae sold as Amaferm Vitaferm (Biozyme Enterprises Inc.).

Examples of anticaking agents and binders include, but are not limited to, synthetic calcium aluminates.

Examples of zootechnlogical additives include for example ammonia control.

Examples of ionophores include monensin such as e.g. Rumensin® from Elanco.

The composition of the invention may further comprise at least one amino acid. Examples of amino acids which are used in ruminant feed include, but are not limited to, lysine, alanine, beta-alanine, threonine, methionine and tryptophan.

### Use of Muramidase to Improve Ruminant Performance

In another aspect, the invention relates to the use of a ruminant feed supplement, a ruminant feed additive or a ruminant feed for improving the Feed Conversion Ratio (FCR) in a ruminant wherein the ruminant feed, ruminant feed supplement or ruminant feed additive comprises one or more muramidases, wherein the muramidase is administered at a level of 1 to 200 mg enzyme protein per kg ruminant feed DM.

In a preferred embodiment, the improvement is compared to a ruminant feed, ruminant feed supplement or ruminant feed additive wherein the muramidase is not present (herein referred to as the negative control).

In one embodiment, the FCR is improved by at least 1%, such as by at least 1.25%, at least 1.5%, at least 1.75% or at least 2.0% compared to the control. In another embodiment, the FCR is improved by between 1% and 5%, such as between 1% and 4%, between 1% and 3%, 1.25% and 2.5%, 1.5% and 2% compared to the control, or any combination of these intervals.

In one embodiment, the muramidase is dosed at a level of 1 to 200 mg enzyme protein per kg ruminant feed DM, such as 5 to 150 mg, 5 to 125 mg, 5 to 100 mg, 5 to 75 mg, 5 to 50 mg, 5 to 40 mg, 10 to 50 or 5 to 25 mg enzyme protein per kg ruminant feed DM, or any combination of these intervals.

In one embodiment, the ruminant is selected from the group consisting of: cattle, cow, dairy cattle, beef cattle, buffalo, calf, goat, sheep, lamb, deer, yak, camel and llama. In a preferred embodiment, the ruminant is selected from the group consisting of: cattle, cow, dairy cattle, beef cattle, buffalo and calf. In a more preferred embodiment, the ruminant is selected from the group consisting of: cattle, dairy cattle and beef cattle.

In one embodiment, the muramidase is provided to the ruminant during any period of time from birth until slaughter. In a preferred embodiment the muramidase is provided to the ruminant on a daily basis. In a further embodiment, the muramidase is provided to the ruminant on a daily basis during the life span of the ruminant.

In one embodiment, the muramidase is provided to growing ruminants. In one embodiment, the muramidase is provided to dairy cattles. In one embodiment, the muramidase is provided to beef cattle in the growing phase of beef cattle production. In one embodiment, the muramidase is provided to beef cattle in the finishing phase of beef cattle production. In a further embodiment, the muramidase is provided to calves in the milk.

In one embodiment, the muramidase is of microbial origin. In a further embodiment, the muramidase is of fungal origin. In an embodiment, the muramidase is obtained or obtainable from the phylum Ascomycota, such as the sub-phylum Pezizomycotina.

In one embodiment, the muramidase comprises one or more domains from a glycoside hydrolase (GH) family selected from the list consisting of GH24, GH25 and novel MUR polypeptides having muramidase activity.

### Preferred Embodiments

The following is a list of preferred embodiments comprised by the invention:

1. A ruminant feed composition, such as a ruminant feed, ruminant feed supplement or ruminant feed additive comprising one or more muramidases, wherein the muramidase is in an amount sufficient for administration at a level of 1 to 200 mg enzyme protein per kg ruminant feed.
2. The ruminant feed composition of embodiment 1, wherein the muramidase is dosed at a level from 1 to 200 mg enzyme protein per kg ruminant feed dry matter.
3. The ruminant feed composition of embodiment 1 or 2, wherein the muramidase is dosed at a level from 5 to 150 mg, from 5 to 125 mg, from 5 to 100 mg, from 5 to 75 mg, from 5 to 50 mg, from 5 to 40 mg, from 10 to 50, from 5 to 25 mg enzyme protein per kg ruminant feed dry matter, or any combination of these intervals.
4. The ruminant feed composition of any one of embodiments 1 to 3, wherein the Energy Corrected Milk (ECM) production of ruminants upon administration is improved by at least 1.0%, preferably at least 1.5% more preferably at least 2.0% compared to control.
5. The ruminant feed composition of any one of embodiments 1 to 4, wherein the ruminant is selected from the group consisting of: cattle, cow, dairy cattle, beef cattle, buffalo, young calf, goat, sheep, lamb, deer, yak, camel and llama.
6. The ruminant feed composition of any one of embodiments 1 to 5, wherein the ruminant is selected from the group consisting of: cattle, dairy cattle and beef cattle.
7. The ruminant feed composition of any one of embodiments 1 to 6, wherein the muramidase is provided to the ruminant during the life span of the ruminant.
8. The ruminant feed composition of any one of embodiments 1 to 7, wherein the muramidase is of microbial origin.
9. The ruminant feed composition of embodiment 8, wherein the muramidase is of fungal origin.
10. The ruminant feed composition of any one of embodiments 1 to 9, wherein the muramidase is obtained or obtainable from the phylum Ascomycota.
11. The ruminant feed composition of any one of embodiments 1 to 10, wherein the muramidase is obtained or obtainable from the subphylum Pezizomycotina.
12. The ruminant feed composition of any one of embodiments 1 to 11, wherein the muramidase comprises one or more domains from a glycoside hydrolase (GH) family selected from the list consisting of GH24, GH25 and novel MUR polypeptides having muramidase activity.
13. The ruminant feed composition of any one of embodiments 1 to 12, wherein the muramidase is selected from the group consisting of:
(a) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59;
(b) a variant of an amino acid sequence selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(c) a fragment of the polypeptide of (a) or (b) that has muramidase activity wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids.
   14. The ruminant feed composition of any one of embodiments 1 to 13, wherein the ruminant feed composition further comprises one or more components selected from the list consisting of: one or more carriers;
   one or more additional enzymes;
   one or more microbes;
   one or more vitamins;
   one or more minerals;
   one or more amino acids;
   one of more organic acids; and
   one or more other feed ingredients.
      15. The ruminant feed composition of any one of embodiments 1 to 14, wherein the muramidase is in granulate form.
      16. The ruminant feed composition of embodiment 15, wherein the granulate is coated.
      17. The ruminant feed composition of embodiment 16 wherein the coating comprises a salt and/or wax and/or a flour.
      18. The ruminant feed composition of any one of embodiments 1 to 17, wherein the muramidase is in a liquid formulation.
      19. The ruminant feed composition of embodiment 18, wherein the liquid formulation is sprayed onto the feed after it has been pelleted.
      20. The ruminant feed composition of any one of embodiments 4 to 19, wherein the control is a ruminant feed composition which does not comprise muramidase.
      21. The ruminant feed composition of any one of embodiments 4 to 20, wherein the control is a ruminant feed composition which does not comprise GH24 muramidase, GH25 muramidase or novel MUR polypeptides having muramidase activity.
      22. The ruminant feed composition of any one of embodiments 4 to 21, wherein the control is a ruminant feed composition comprising Hen Egg White Lysozyme (HEWL).
      23. The ruminant feed composition of any one of embodiments 4 to 21, wherein the control is monensin.
      24. The ruminant feed composition of any one of embodiments 1 to 23, wherein the ruminant feed composition comprising muramidase is administered to a ruminant selected from the group consisting of: A growing ruminant, a dairy cattle, a beef cattle in the growing phase of beef cattle production, a beef cattle in the finishing phase of beef cattle production, and a calf.
      25. The ruminant feed composition of any one of embodiments 1 to 24, wherein the ruminant feed composition comprising muramidase is administered to a growing ruminant.
      26. The ruminant feed composition of any one of embodiments 1 to 24, wherein the ruminant feed composition comprising muramidase is administered to a dairy cattle.
      27. The ruminant feed composition of any one of embodiments 1 to 24, wherein the ruminant feed composition comprising muramidase is administered to a beef cattle in the growing phase of beef cattle production.
      28. The ruminant feed composition of any one of embodiments 1 to 24, wherein the ruminant feed composition comprising muramidase is administered to a beef cattle in the finishing phase of beef cattle production.
      29. The ruminant feed composition of any one of embodiments 1 to 24, wherein the ruminant feed composition comprising muramidase is administered to a calf.
      30. A method of improving the Energy Corrected Milk (ECM) production of a ruminant comprising administering to the ruminant a ruminant feed composition according to any one of embodiments 1 to 29.
      31. A method for increasing dry matter digestibility (DMd) of a ruminant feed, ruminant feed supplement or ruminant feed additive comprising the steps of: a) providing at least one muramidase; b) providing a ruminant feed, ruminant feed supplement or ruminant feed additive suitable for a ruminant animal; c) applying the muramidase to the ruminant feed, ruminant feed supplement or ruminant feed additive to form a ruminant feed composition; and d) feeding the ruminant feed composition to the ruminant animal, whereby an increase in dry matter digestibility is effected.
      32. The method of embodiment 31, wherein DMd is measured according to example 2.
      33. The method of any one of embodiments 31 to 32, wherein DMd is increased compared to DMd in feed prepared as in embodiment 31 but without muramidase added in step c).
      34. The method of any one of embodiments 31 to 33, wherein the production of volatile fatty acids (VFA) in the rumen is increased compared to the production of VFA in the rumen of a ruminant not fed with a muramidase.
      35. The method of any one of embodiments 31 to 34, wherein the production of propionate in the rumen is increased compared to the production of propionate in the rumen of a ruminant not fed with a muramidase.
      36. The method of any one of embodiments 31 to 35, wherein the production of acetate in the rumen is increased compared to the production of acetate in the rumen of a ruminant not fed with a muramidase.
      37. The method of any one of embodiments 31 to 36, wherein the muramidase is dosed at a level from 1 to 310 mg enzyme protein per kg ruminant feed dry matter.
      38. The method of any one of embodiments 31 to 37, wherein the muramidase is dosed at a level from 5 to 150 mg, from 5 to 125 mg, from 5 to 100 mg, from 5 to 75 mg, from 5 to 50 mg, from 5 to 40 mg, from 10 to 50, from 5 to 25 mg enzyme protein per kg ruminant feed dry matter, or any combination of these intervals.
      39. The method of any one of embodiments 31 to 38, wherein the Energy Corrected Milk (ECM) production of ruminants upon administration is improved by at least 1.0%, preferably at least 1.5% more preferably at least 2.0% compared to control.
      40. The method of any one of embodiments 31 to 39, wherein the ruminant is selected from the group consisting of: cattle, cow, dairy cattle, beef cattle, buffalo, young calf, goat, sheep, lamb, deer, yak, camel and llama.
      41. The method of any one of embodiments 31 to 40, wherein the ruminant is selected from the group consisting of: cattle, dairy cattle and beef cattle.
      42. The method of any one of embodiments 31 to 41, wherein the muramidase is provided to the ruminant during the life span of the ruminant.
      43. The method of any one of embodiments 31 to 42, wherein the muramidase is of microbial origin.
      44. The method of any one of embodiments 31 to 42, wherein the muramidase is of fungal origin.
      45. The method of any one of embodiments 31 to 44, wherein the muramidase is obtained or obtainable from the phylum Ascomycota.
      46. The method of any one of embodiments 31 to 45, wherein the muramidase is obtained or obtainable from the subphylum Pezizomycotina.
      47. The method of any one of embodiments 31 to 46, wherein the muramidase comprises one or more domains from a glycoside hydrolase (GH) family selected from the list consisting of GH24, GH25 and novel MUR polypeptides having muramidase activity.
      48. The method of any one of embodiments 31 to 47, wherein the muramidase is selected from the group consisting of:
(a) a polypeptide having at least 50%, e.g., at least 60%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59;
(b) a variant of an amino acid sequence selected from the group consisting of: SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59 wherein the variant has muramidase activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 positions;
(c) a fragment of the polypeptide of (a) or (b) that has muramidase activity wherein the fragment comprises at least 170 amino acids, such as at least 175 amino acids, at least 177 amino acids, at least 180 amino acids, at least 185 amino acids, at least 190 amino acids, at least 195 amino acids or at least 200 amino acids.
   49. The method of any one of embodiments 31 to 48, wherein the muramidase is in granulate form.
   50. The method of embodiment 49, wherein the granulate is coated.
   51. The method of embodiment 50 wherein the coating comprises a salt and/or wax and/or a flour.
   52. The method of any one of embodiments 31 to 51, wherein the muramidase is in a liquid formulation.
   53. The method of embodiment 52, wherein the liquid formulation is sprayed onto the feed after it has been pelleted.
   54. The method of any one of embodiments 39 to 53, wherein the control is a ruminant feed composition which does not comprise muramidase.
   55. The method of any one of embodiments 39 to 53, wherein the control is a ruminant feed composition which does not comprise GH24 muramidase, GH25 muramidase or novel MUR polypeptides having muramidase activity.
   56. The method of any one of embodiments 39 to 55, wherein the control is a ruminant feed composition comprising Hen Egg White Lysozyme (HEWL).
   57. The method of any one of embodiments 39 to 55, wherein the control is monensin.
   58. The method of any one of embodiments 31 to 57, wherein the ruminant feed composition comprising muramidase is administered to a ruminant selected from the group consisting of: A growing ruminant, a dairy cattle, a beef cattle in the growing phase of beef cattle production, a beef cattle in the finishing phase of beef cattle production, and a calf.
   59. The method of any one of embodiments 31 to 58, wherein the ruminant feed composition comprising muramidase is administered to a growing ruminant.
   60. The method of any one of embodiments 31 to 58, wherein the ruminant feed composition comprising muramidase is administered to a dairy cattle.
   61. The method of any one of embodiments 31 to 58, wherein the ruminant feed composition comprising muramidase is administered to a beef cattle in the growing phase of beef cattle production.
   62. The method of any one of embodiments 31 to 58, wherein the ruminant feed composition comprising muramidase is administered to a beef cattle in the finishing phase of beef cattle production.
   63. The method of any one of embodiments 31 to 58, wherein the ruminant feed composition comprising muramidase is administered to a calf.

### EXAMPLES

The muramidases were cloned, expressed, characterised and tested for muramidase activity as described in WO 2013/076253.

### Example 1—Effect of Muramidase on Ruminal Fermentation Gas Production and Feed Dry Matter Digestibility

### Material and Methods:

The in vitro fermentation model was adapted from Menke and Steingass, (Menke KH, Steingass H. (Estimation of the energetic feed value obtained from chemical analysis and in vitro gas production using rumen fluid. Anim Res Dev. (1988) 28:7-55). The experimental design included treatments of negative control (NC, just feed and ruminal buffer solution), positive control (PC, feed and ruminal buffer solution added a commercial ruminal modifier (Monensin)), muramidase (SEQ ID NO: 3, 6, 9, 12, 15, 18, 21, 24 and 27, negative control with muramidases), and blanks (only ruminal buffer solution), Table 1. The trial was repeated 2 times (trial 1, and trial 2) to increase the power of the overall study.

TABLE 1

| | | | | | Dosage |
|---|---|---|---|---|---|
| | N | | Feed | | mg EP/kg |
| Treatment | Trial 1 | Trial 2 | g | Enzyme class | feed |
| Blank | 3 | 3 | — | | |
| NC | 4 | 6 | 0.5 | | |
| PC (Monensin) | 3 | 4 | 0.5 | Non-enzyme product | 22 |
| SEQ ID NO: 3 | 3 | 3 | 0.5 | novel MUR polypeptides | 50 |
| | | | | having muramidase activity | |
| SEQ ID NO: 6 | 3 | 3 | 0.5 | novel MUR polypeptides | 50 |
| | | | | having muramidase activity | |
| SEQ ID NO: 9 | 3 | 3 | 0.5 | novel MUR polypeptides | 50 |
| | | | | having muramidase activity | |
| SEQ ID NO: 12 | 3 | 3 | 0.5 | GH25 | 50 |
| SEQ ID NO: 15 | 3 | 3 | 0.5 | GH25 | 50 |
| SEQ ID NO: 18 | 3 | 3 | 0.5 | GH25 | 50 |
| SEQ ID NO: 21 | 3 | 3 | 0.5 | GH24 | 25 |
| SEQ ID NO: 24 | 3 | 3 | 0.5 | GH24 | 25 |
| SEQ ID NO: 27 | 3 | 3 | 0.5 | novel MUR polypeptides | 50 |
| | | | | having muramidase activity | |

The method was based on incubation of ruminant feed in a buffer and ruminal fluid solution for 48 h period while obtaining the cumulative gas production and digestibility of the feed DM. The buffer mineral solution used was prepared according to Menke and Steingass (1988), and was heated in a water bath at 39° C. and purged continuously with CO₂ for 60 minutes. Sodium sulphite was used as reducing agent in the buffer solution (0.33 g/I solution). Rumen fluid was collected from 2 Jersey heifers (housed at the experimental farm of Copenhagen University, Denmark). Rumen fluid was collected through the ruminal cannula and poured into two thermal flasks preheated to 39.0±0.5° C. and immediately transferred to the laboratory. The rumen fluid was filtered through 3 layers of cheesecloth to eliminate feed particles and mixed with the buffer mineral solution in a ratio 1 to 2 (Menke and Steingass, 1988).

The ruminal fluid and buffer solution was dispensed 90 mL into Duran flask fitted with a lid equipped with wireless pressure transducer and gas valve (Ankom RF Gas Production System, Ankom Technology®, Macedon, N.Y., USA). Following filling with 0.5000±0.0010 g of feed sample (corn silage, soy bean meal in 2:1 ratio on a dry matter basses) and preheated overnight at 39° C. The Duram flasks were dosed with 1.00 mL of treatment solution (either water or murimidase solution, or positive control solution) were administrated and mixed before closing the flasks. The enzyme solutions (A to I) were all produces by Novozymes and were dosed according to mg enzyme protein per kg feed. Table 1. As PC was used Monensin (Monensin sodium salt hydrate, lot #BCBR9717V, Sigma-Aldrich, Buchs Switzerland) at the concentration 22 mg/kg feed, Table 1. All procedures were performed under continuous purge with CO₂. The headspace of each flask after filling was 41 mL. Fermentation gas was automatically released from the flasks by the gas valve whenever gas pressure increased 0.75 PSI. The pressure difference related to opening the gas valve were used calculate the cumulated (ΔP) gas production using the ideal gas law: GP=(ΔP/Po)·Vo(1) where: ΔP is the cumulated pressure change (kPa) in the bottle headspace; Vo is the bottle headspace volume (41 mL), Po is the atmospheric pressure. Data of gas production were expressed as ml/g DM incubated under standard pressure and was not included in the current example but was uses to ensure sufficient fermentation activity. Dry matter following the fermentation was measured and related to initial dry matter in the flasks to determine dry matter digestibility as (dry matter before−dry matter after)/dry matter before). Data was expressed as the relative improvement of DMd compared to control when taking the background fermentation of the blanks into account. Data was statistically analyzed by the ANOVA procedure resting within JMP 12.1.0 (SAS Institute Inc.) and presented as LS-means, the initial model included treatment, trial and interactions, and was after model evaluation reduced to the main effect of treatment.

### Results

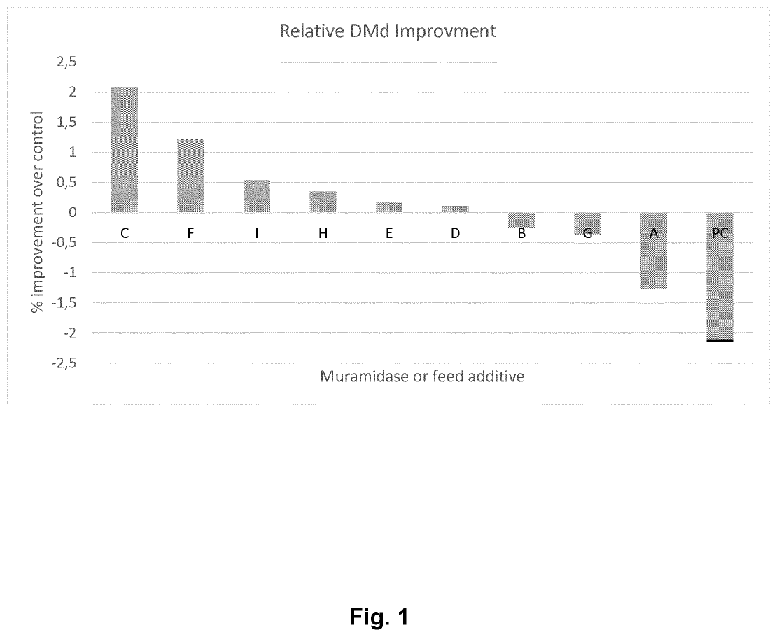

The result of relative DMd is presented in FIG. 1, and shows that the addition of muramidase increases the 48 h in vitro ruminal digestion of feed dry matter compared to the positive control (Monensin). Data also shows that addition of some muramidases can increase the DMd of ruminant feed compared to NC.

### Conclusions

In conclusion, across two replications of the same study a range of muramidases from 3 different classes showed an improvement in ruminal dry matter digestibility over the commonly used ruminal additive Monensin. Data also showed that most muramidases improves the ruminal feed dry matter digestibility over NC.

### Example 2—Dosage Response Effect of 5 Muramidase on Ruminal Fermentation and Feed Dry Matter Digestibility

### Summary:

In vitro ruminal fermentation using ruminal fluid and buffer showed that muramidase can increase the dry matter digestibility and increase the production of ruminal acetate, propionate, total VFA and increase the pool of total carbon in ruminal VFA.

### Material and Methods:

The trial was performed by Alimetrics (Alimetrics Ltd, Koskelontie 19B, FIN-02920 Espoo, Finland) using 120 mL serum bottles as fermentation vessels. The fermentation preparation and procedure is presented in Kettunen et al., (Kettunen H., J. Vuorenmaa, D. Gaffney and J. Apajalahti (2016): Yeast hydrolysate product enhances ruminal fermentation in vitro. J. App. Anim. Nutr., Vol. 4; e1; page 1 of 7). In short, the method is as follows: All bottles were initially filled with 1 g of feed DM composed from corn silage, barley meal, soybean meal, at the amounts of 0.5, 0.4, and 0.1 g DM, respectively. Subsequently, the bottles were flushed with CO₂ passed through a hot copper catalyst for 02 scavenging and sealed with thick butyl rubber stopper. Anaerobic, reduced and temperature adjusted (+38° C.) artificial saliva buffer solution (Modified from Agriculture Handbook No. 379, (USDA, Washington, D.C., 1975), volume of 37.65 ml), freshly strained rumen fluid (5% inoculum; volume of 2 ml), and test compounds (volume of 0.35 ml) was introduced into the fermentation vessels under the oxygen-free CO₂ flow. Following rumen fluid collection from a ruminal cannulated cow. Rumen fluid was pumped directly from the rumen into a preheated thermos and immediately closed, and transported to Alimetrics laboratory. The rumen fluid was used for inoculation within 2 hours of harvest. To ensure that both liquid and particle associated ruminal microbes were present in the inoculum both fractions were obtained. Liquid and solid fractions were strained through a metal mesh (grid size 3×3 mm) under anaerobic conditions prior to buffer dilution.

Following inoculation with rumen fluid, buffer and test solution the vessels were sealed with the butyl rubber septum's. All fermentation vessels were inoculated in random order to prevent the possible block effects. Inoculation time for each fermentation vessel was registered and was taken into account op on each sampling to ensure that duration of the fermentation was the same for each sample. The fermentation was continued for 12 h in a gyratory shaker at +38° C. Fermentation gas produced was measured and relived by collection of the fermentation gas in syringes at time 3, 6, 9, and 12 h. Feed residues were measured after 12 h fermentation.

The trial design was a dosage response, complete randomized design with 28 treatments using 150 fermentation vessels according to Table 2. The 25 muramidase solutions were diluted to the concentration needed in enzyme buffer (BSA, Tween 20, acetate-buffer, Calcium, NaOH, adjusted to pH 6). Negative control was supplemented with the same buffer solution that was used for diluting the enzymes to reach the same liquid volume as the supplemented fermentation vessels. The ionophore Monensin (Monensin sodium salt, 90-95% TLC (Sigma Aldrich; Product code: M5273-1G)) was chosen as a positive control because it is a ruminants feed additive that affects volatile fatty acids (VFA) production.

TABLE 2
treatments, dosage and study design

| | | | | 1 | 2 | 3 | 4 | 5 |
|---|---|---|---|---|---|---|---|---|
| Dosage | N/dosage | SEQ ID | Enzyme class | mg/40 ml | | | | |
| NC | 15 | | | | | | | |
| PC (Monensin) | 5 | | Non-enzyme product | 0.05 | 0.25 | | | |
| A | 5 | SEQ ID NO: 28 | GH24 | 0.05 | 0.1 | 0.2 | 0.4 | 0.8 |
| B | 5 | SEQ ID NO: 21 | GH24 | 0.05 | 0.1 | 0.2 | 0.4 | 0.8 |
| C | 5 | SEQ ID NO: 12 | GH25 | 0.05 | 0.1 | 0.2 | 0.4 | 0.8 |
| D | 5 | SEQ ID NO: 18 | GH25 | 0.05 | 0.1 | 0.2 | 0.4 | 0.8 |
| E | 5 | THX3038 | novel MUR polypeptides | 0.05 | 0.1 | 0.2 | 0.4 | 0.8 |
| | | SEQ ID NO: 9 | having muramidase activity | | | | | |

### Sample Collection

Liquid samples obtained at 12 h were analyzed for the concentration of VFA (acetate, propionate, butyrate, and valerate) by GC-FID using a glass column packed with 80/120 carbopack B-DA/4% Carbowax stationary and helium as a carrier gas (Kettunen H., J. Vuorenmaa, T. Rinttilä, H. Grönberg, E. Valkonen and J. Apajalahti (2015): Natural resin acid-enriched composition as a modulator of intestinal microbiota and performance enhancer in broiler chicken. J. App. Anim. Nutr., Vol. 3; e2; page 1 of 9).

Digestibility of dry matter was quantified by determination of dry matter in the original feed matrix and in all fermentation vessels after 12 h of fermentation. Feed residues from the fermentation vessels were obtained by filtration tared sintered glass filters, washed with water and dried at 105° C. for 12 hours. Finally, the glass filters are weighed for the residual feed dry matter.

### Calculations and Statistic

Dry matter digestibility was calculated at the ratio between feed residues after end fermentation and the dry matter fed. The sum of VFA were calculated as the sum of acetate, propionate, butyrate and valerate, and the summed carbon in VFA was calculated by assigning the respective number of carbon atoms in acetate, propionate, butyrate, and valerate, 2, 3, 4, and 5, respectively. The relative improvement compared to NC was calculated for DMd, and the concentration of acetate, propionate, butyrate, total VFA, and total carbon in VFA.

Data was analyzed for linear and quadratic effects using the algorithm for linear regression resting in the proc mixed procedure of SAS (SAS institute). Data presented are linear regression estimates and standard error, as well as least square means and standard error, unless otherwise stated.

### Results:

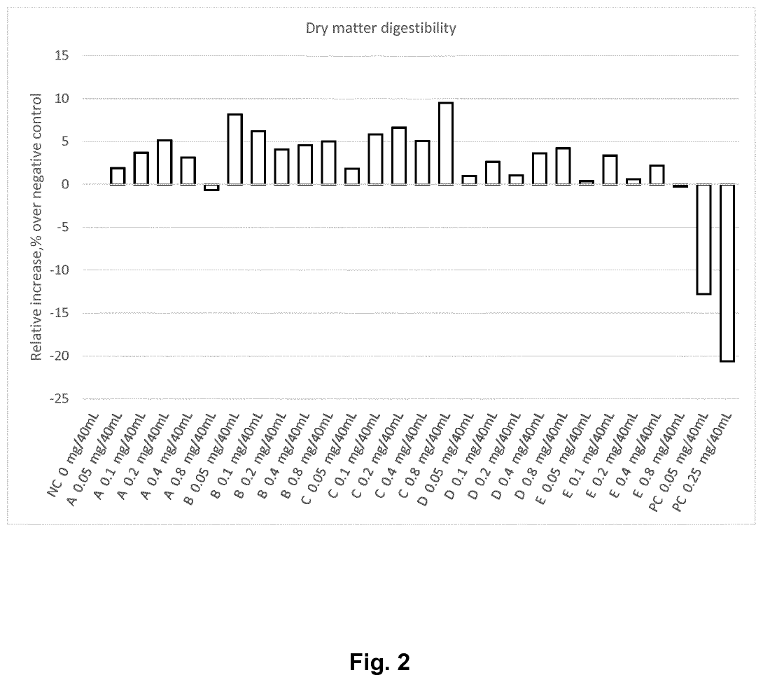

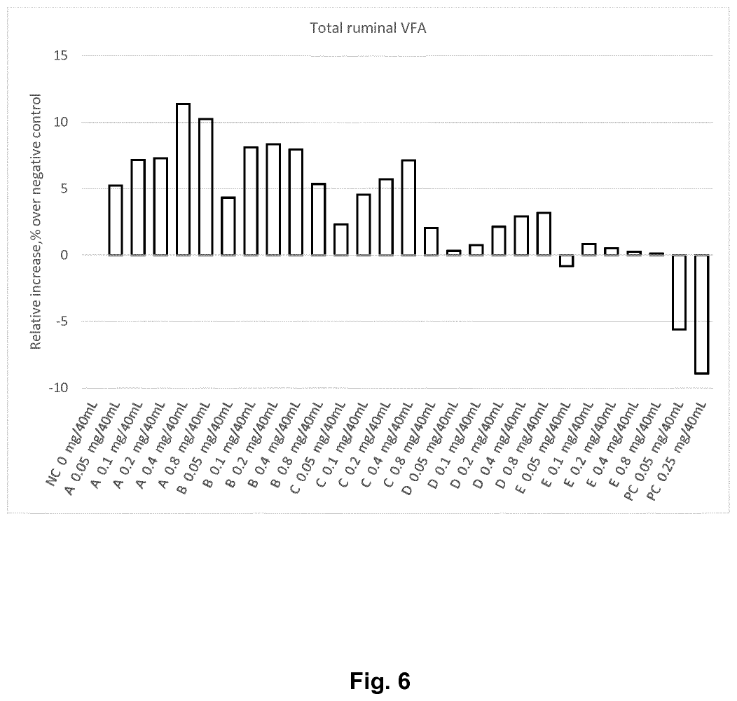

Data shows that muramidases can affect ruminal dry matter digestibility and VFA production in vitro FIG. 2-7. All muramidase treatments increased the DMd when compared to control. The greatest improvement was for treatment C (9.5%) when the fermentation was supplemented with 0.8 mg/40 mL.

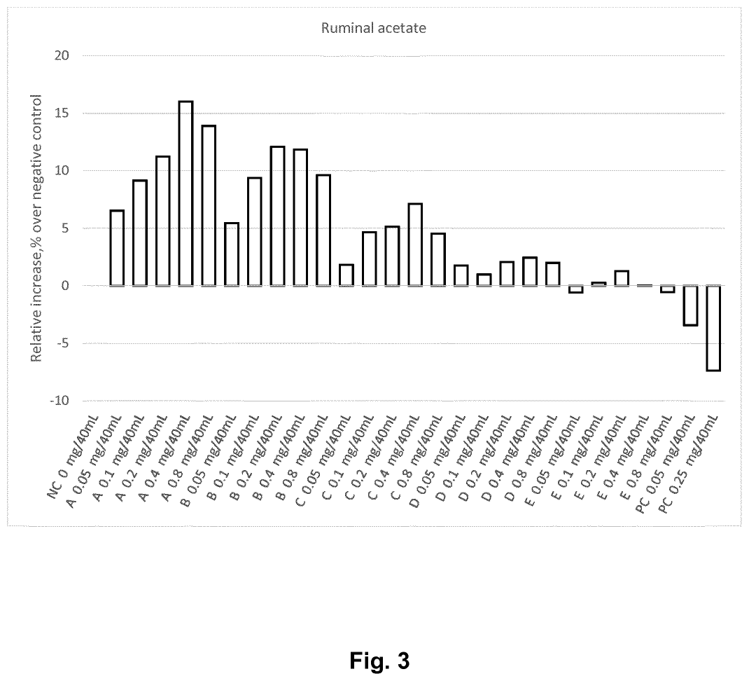

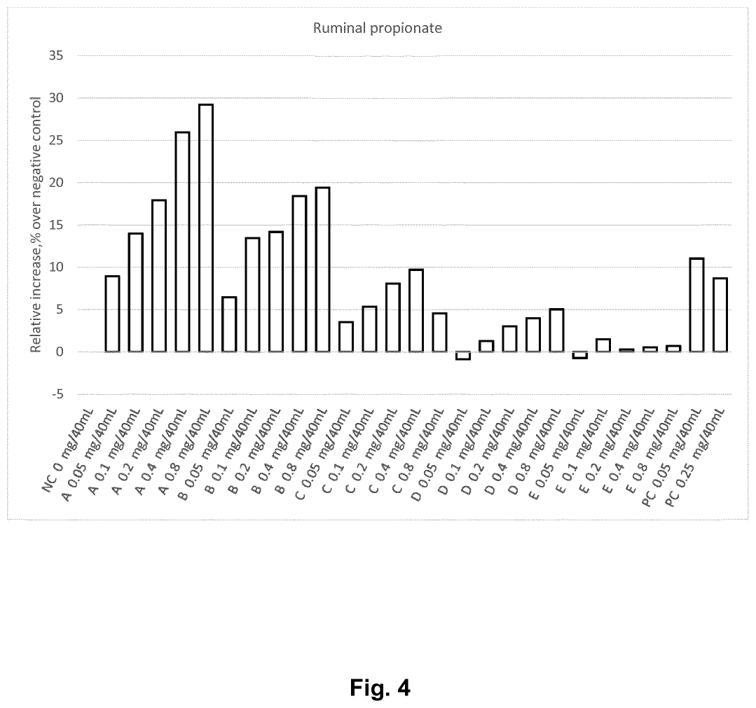

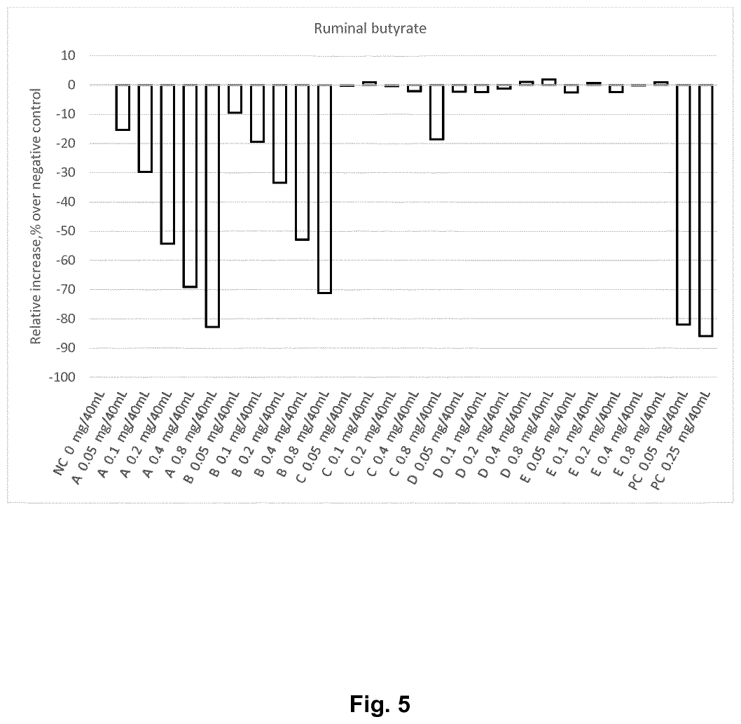

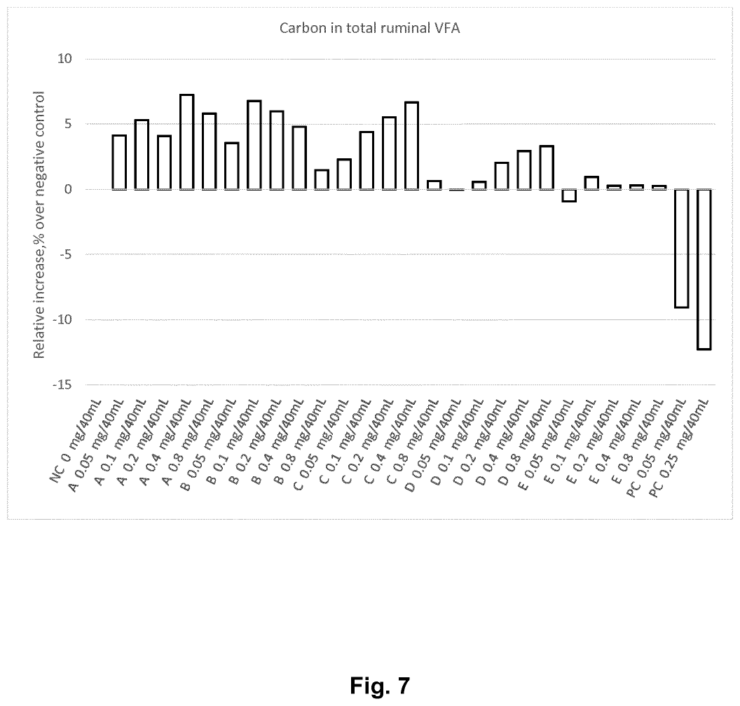

All muramidase treatments increased the ruminal acetate and propionate production numerically compared to control. Acetate was increased 16.0% when treatment A was supplemented at 0.4 mg/40 mL, at the same dosage propionate was increased 24%, resulting in an 11% increase in total VFA production FIGS. 3 and 4 respectively. Treatment A and B also decreased ruminal butyrate production compared to control FIG. 5. However, carbon in VFA was still affected positive despite the reduction in butyrate, the improvement relative to NC was up to 7.3%, FIG. 7.

Linear regression analysis identified treatments A, B and C as the most potent in increasing the ruminal acetate and propionate production. Linear regression analysis also identified treatment A, B and C as the most potent in reducing ruminal butyrate production. The effect on acetate and propionate by the dosage of treatment A, B, and C were quadratic. Total ruminal VFA production and total carbon in ruminal VFA were affected quadratic by dosage of muramidase for treatment C.

### Conclusion:

In conclusion data shows that muramidase can increase the dry matter digestibility and increase the production of ruminal acetate, propionate, total VFA and increase the pool of total carbon in ruminal VFA in vitro.

### Example 3—Effect of 13 Muramidases on Ruminal Fermentation and Feed Dry Matter Digestibility In Vitro

### Summary:

A fermentation study was performed using 13 muramidases from glycoside hydrolase (GH) families GH24, GH25, and novel MUR polypeptides having muramidase activity, the number of enzymes tested was 5, 4, and 4, respectively. The fermentation was performed in vitro using ruminal fluid and artificial saliva solution in 120 mL fermentors. The hypothesis was that muramidases can increase the production of ruminal fermentation products.

In conclusion data shows that muramidases from glycoside hydrolase (GH) families GH24, GH25, and novel MUR polypeptides having muramidase activity can affect ruminal dry matter digestibility, and ruminal fermentation, by increasing the production of propionate, total volatile fatty acids and total carbon in volatile fatty acids.

### Material and Methods:

The trial was performed by Alimetrics (Alimetrics Ltd, Koskelontie 19B, FIN-02920 Espoo, Finland) using 120 mL serum bottles as fermentation vessels. The fermentation preparation and procedure is presented in Kettunen et al., (Kettunen H., J. Vuorenmaa, D. Gaffney and J. Apajalahti (2016): Yeast hydrolysate product enhances ruminal fermentation in vitro. J. App. Anim. Nutr., Vol. 4; e1; page 1 of 7). In short, the method is as follows: All bottles were initially filled with 1 g of feed DM composed from corn silage, barley meal, soybean meal, at the amounts of 0.5, 0.4, and 0.1 g DM, respectively. Subsequently, the bottles were flushed with CO₂ passed through a hot copper catalyst for O₂ scavenging and sealed with thick butyl rubber stopper. Anaerobic, reduced and temperature adjusted (+38° C.) artificial saliva buffer solution (Modified from Agriculture Handbook No. 379, (USDA, Washington, D.C., 1975), volume of 37.65 ml), freshly strained rumen fluid (5% inoculum; volume of 2 ml), and test compounds (volume of 0.35 ml) was introduced into the fermentation vessels under the oxygen-free CO₂ flow. Following rumen fluid collection from a ruminal cannulated cow. Rumen fluid was pumped directly from the rumen into a preheated thermos and immediately closed, and transported to Alimetrics laboratory. The rumen fluid was used for inoculation within 2 hours of harvest. To ensure that both liquid and particle associated ruminal microbes were present in the inoculum both fractions were obtained. Liquid and solid fractions were strained through a metal mesh (grid size 3×3 mm) under anaerobic conditions prior to buffer dilution.

Following inoculation with rumen fluid, buffer and test solution the vessels were sealed with the butyl rubber septum's. All fermentation vessels were inoculated in random order to prevent the possible block effects. Inoculation time for each fermentation vessel was registered and was taken into account op on each sampling to ensure that duration of the fermentation was the same for each sample. The fermentation was continued for 12 h in a gyratory shaker at +38° C. Fermentation gas produced was measured and relived by collection of the fermentation gas in syringes at time 3, 6, 9, and 12 h. Feed residues were measured after 12 h fermentation.

The trial design was a dosage response, complete randomized design with 28 treatments using 150 fermentation vessels according to table 3. The 13 muramidase solutions were diluted to the concentration needed in enzyme buffer (BSA, Tween 20, acetate-buffer, Calcium, NaOH, adjusted to pH 6). Negative control was supplemented with the same buffer solution that was used for diluting the enzymes to reach the same liquid volume as the supplemented fermentation vessels. The ionophore Monensin (Monensin sodium salt, 90-95% TLC (Sigma Aldrich; Product code: M5273-1G)) was chosen as a positive control because it is a ruminants feed additive that affects volatile fatty acids (VFA) production.

TABLE 3
treatments, dosage and study design

| | | | | 1 | 2 | 3 | 4 | 5 |
|---|---|---|---|---|---|---|---|---|
| Dosage | N/dosage | SEQ ID | Enzyme class | mg/40 ml | | | | |
| NC | 15 | | | | | | | |
| PC (Monensin) | 5 | | Non-enzyme product | 0.01 | | 0.1 | | |
| A | 5/25 | SEQ ID NO: 28 | GH24 | 0.025 | 0.05 | 0.1 | 0.2 | 0.4 |
| B | 5/25 | SEQ ID NO: 21 | GH24 | 0.025 | 0.05 | 0.1 | 0.2 | 0.4 |
| C | 5/25 | SEQ ID NO: 12 | GH25 | 0.025 | 0.05 | 0.1 | 0.2 | 0.4 |
| D | 5 | SEQ ID NO: 29 | GH24 | | | | 0.2 | |
| E | 5 | SEQ ID NO: 30 | GH24 | | | | 0.2 | |
| F | 5 | SEQ ID NO: 31 | GH24 | | | | 0.2 | |
| G | 5 | SEQ ID NO: 32 | GH25 | | | | 0.2 | |
| H | 5 | SEQ ID NO: 33 | GH25 | | | | 0.2 | |
| I | 5 | SEQ ID NO: 34 | GH25 | | | | 0.2 | |
| J | 5 | SEQ ID NO: 35 | novel MUR polypeptides | | | | 0.2 | |
| | | | having muramidase activity | | | | | |
| K | 5 | SEQ ID NO: 36 | novel MUR polypeptides | | | | 0.2 | |
| | | | having muramidase activity | | | | | |
| L | 5 | SEQ ID NO: 36 | novel MUR polypeptides | | | | 0.2 | |
| | | | having muramidase activity | | | | | |
| M | 5 | SEQ ID NO: 37 | novel MUR polypeptides | | | | 0.2 | |
| | | | having muramidase activity | | | | | |

### Sample Collection

Liquid samples obtained at 12 h were analyzed for the concentration of VFA (acetate, propionate, butyrate, and valerate) by GC-FID using a glass column packed with 80/120 carbopack B-DA/4% Carbowax stationary and helium as a carrier gas (Kettunen H., J. Vuorenmaa, T. Rinttilä, H. Grönberg, E. Valkonen and J. Apajalahti (2015): Natural resin acid-enriched composition as a modulator of intestinal microbiota and performance enhancer in broiler chicken. J. App. Anim. Nutr., Vol. 3; e2; page 1 of 9).

Digestibility of dry matter was quantified by determination of dry matter in the original feed matrix and in all fermentation vessels after 12 h of fermentation. Feed residues from the fermentation vessels were obtained by filtration tared sintered glass filters, washed with water and dried at 105° C. for 12 hours. Finally, the glass filters are weighed for the residual feed dry matter.

### Calculations and Statistic

Dry matter digestibility (DMd) was calculated at the ratio between feed residues after end fermentation and the dry matter fed. The sum of VFA were calculated as the sum of acetate, propionate, butyrate and valerate, and the summed carbon in VFA was calculated by assigning the respective number of carbon atoms in acetate, propionate, butyrate, and valerate 2, 3, 4 and 5, respectively. Data was calculated as the relative improvement compared to NC for DM digestibility, and the concentration of acetate, propionate, butyrate, summed VFA, and total carbon in VFA.

Data was split in two data sets. Data set one, included data from treatment NC and all muramidase treatments at dosage 0.20 mg/40 ml, and PC at dosage 0.01 mg/40 mL, for analysis using the mixed procedure of SAS (SAS institute) and treatment as main effect. Data set two, included data from treatment A, B and C, for all 5 dosages, for analyzed of linear and quadratic effects using the algorithm for linear regression resting in the proc mixed procedure of SAS (SAS institute). Data presented are linear regression estimates and standard error, as well as least square means and standard error, unless otherwise stated.

### Results:

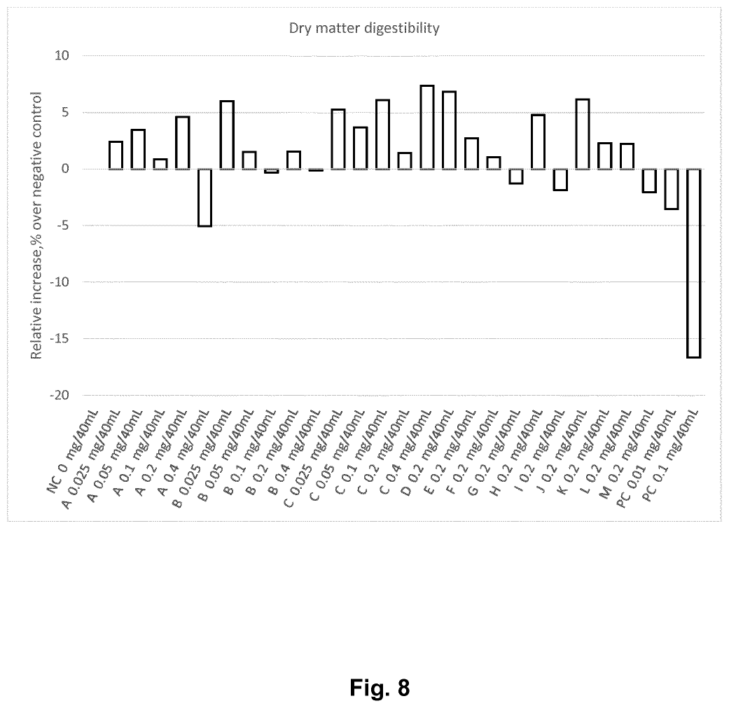

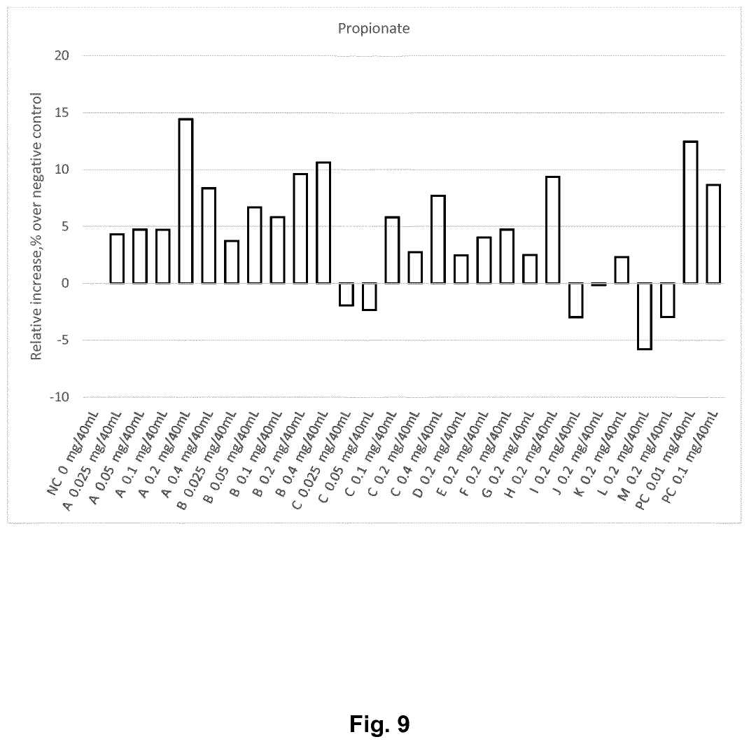

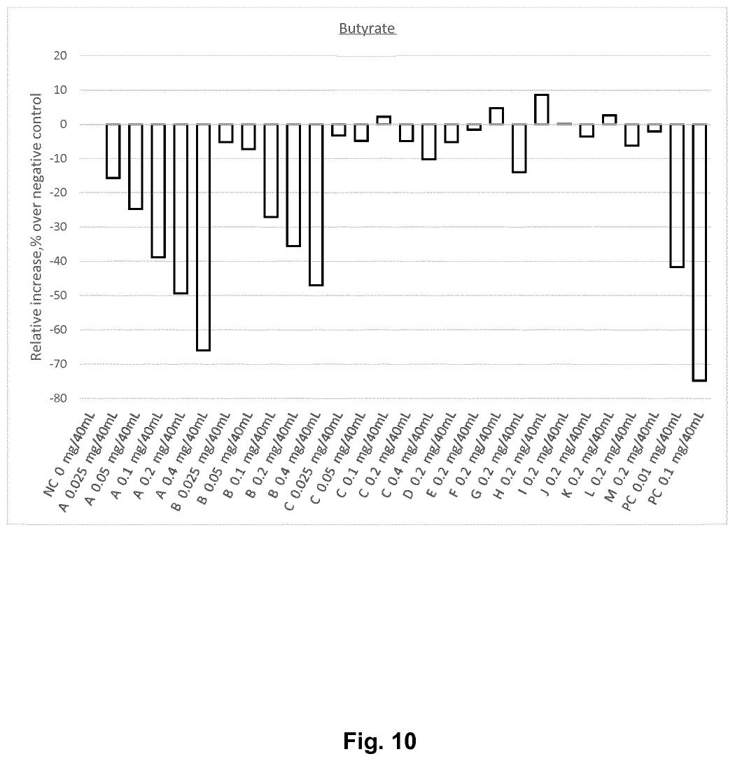

Data of the current study shows that muramidases can affect ruminal DMd and VFA production in vitro, table 4 and FIG. 8-10. The difference in DMd between the unsupplemented NC and the muramidase treatments were positive for 10 out of 13 muramidases, when evaluated at the same dosage (0.2 mg/40 mL). The maximal improvement in DMd 8.6% FIG. 8. The increasing response on DMd was evenly distributed on the 3 glycoside hydrolase (GH) families (GH24, GH 25, and novel MUR polypeptides having muramidase activity defined herein, with 5/5, 2/4, and 3/4, respectively). Data also shows a clear effect of muramidase supplementation on propionate production. The production of propionate increased compared to NC for 9 out of 13 muramidases when evaluated at the same dosage (0.2 mg/40 mL). The increase in propionate production was up to 14.4% FIG. 9. The increasing response on propionate production was divided on the glycoside hydrolase (GH) families GH24, GH25 and novel MUR polypeptides having muramidase activity, in that order, (5/5, 3/4, and 1/4, respectively). The muramidase treatments decreased the butyrate production compared to control for 9 out of 13 muramidases when evaluated at the same dosage (0.2 mg/40 mL). The decrease in butyrate production was up to 49.3% FIG. 10. The decreasing response on butyrate production was divided on the glycoside hydrolase (GH) families GH24, GH25 and novel MUR polypeptides having muramidase activity, (4/5, 2/4, and 3/4, respectively). The effect of supplementing 0.2 mg muramidase/40 mL increased the overall production of VFA and total carbon in VFA for 8 out of 13 treatments when compared to control table 4. The increase total VFA and total carbon in VFA was divided on the glycoside hydrolase (GH) families GH24, GH25 and novel MUR polypeptides having muramidase activity, in that order, (4/5, 2/4, and 2/4, respectively).

For the three muramidases A, B, and C, regression analysis was performed. The regression analysis showed that propionate production increased with increasing dosage of muramidase, and that butyrate production decreased with increasing muramidase dosage, also observed from FIG. 9 and 10, respectively.

TABLE 4
Effect of 13 muramidases (dosage 0.2 mg/40 ml) on
dry matter digestibility, and VFA production (mmol/L)
after 12 h of ruminal fermentation in vitro

| | | | | | Total | Carbon |
|---|---|---|---|---|---|---|
| | DMd | Acetate | Propionate | Butyrate | VFA | in VFA |
| NC | 0.57 | 48.5 | 41.2 | 6.62 | 97 | 250 |
| A | 0.59 | 49.8 | 47.2 | 3.35 | 101 | 256 |
| B | 0.58 | 49.5 | 45.2 | 4.27 | 99 | 254 |
| C | 0.58 | 48.8 | 42.4 | 6.3 | 98 | 252 |
| D | 0.61 | 48.1 | 42.2 | 6.27 | 97 | 250 |
| E | 0.58 | 48.7 | 42.9 | 6.52 | 99 | 255 |
| F | 0.57 | 49.3 | 43.2 | 6.93 | 100 | 258 |
| G | 0.56 | 48.6 | 42.3 | 5.70 | 97 | 249 |
| H | 0.59 | 51.8 | 45.1 | 7.19 | 105 | 270 |
| I | 0.56 | 47.1 | 40 | 6.62 | 94 | 243 |
| J | 0.6 | 47.9 | 41.2 | 6.38 | 96 | 247 |
| K | 0.58 | 49.3 | 42.2 | 6.79 | 99 | 255 |
| L | 0.58 | 46.1 | 38.8 | 6.21 | 92 | 236 |
| M | 0.56 | 48.3 | 40 | 6.48 | 95 | 245 |
| PC | 0.55 | 47.5 | 46.4 | 3.86 | 98 | 251 |
| SEM | 0.016 | 1.38 | 1.47 | 0.21 | 3 | 7.8 |

### Conclusion:

In conclusion data shows that muramidases from the glycoside hydrolase (GH) families GH24, GH25, and novel MUR polypeptides having muramidase activity can affect ruminal dry matter digestibility, and ruminal fermentation, by increasing the production of propionate, total VFA and total carbon in VFA.

### Example 4—Effect of 22 Muramidases on Ruminal Fermentation and Feed Dry Matter Digestibility In Vitro

### Summery:

A fermentation study was performed using muramidases from glycoside hydrolase (GH) families GH24, GH25, and novel MUR polypeptides having muramidase activity, the number of enzymes tested was 9, 8, and 7, respectively. The fermentation was performed in vitro using ruminal fluid and artificial saliva solution in 120 mL fermentores. The hypothesis was that muramidases can increase the production of ruminal fermentation products.

In conclusion ruminal fermentation improved by supplementing muramidases from the three glycoside hydrolase (GH) families GH24, GH25 and novel MUR polypeptides having muramidase activity. The improvement was observed as an increased production of total volatile fatty acids, acetate and propionate.

### Material and Methods:

The trial was performed by Alimetrics (Alimetrics Ltd, Koskelontie 19B, FIN-02920 Espoo, Finland) using 120 mL serum bottles as fermentation vessels. The fermentation preparation and procedure is presented in Kettunen et al., (Kettunen H., J. Vuorenmaa, D. Gaffney and J. Apajalahti (2016): Yeast hydrolysate product enhances ruminal fermentation in vitro. J. App. Anim. Nutr., Vol. 4; e1; page 1 of 7). In short, the method is as follows: All bottles were initially filled with 1 g of feed DM composed from corn silage, barley meal, soybean meal, at the amounts of 0.5, 0.4, and 0.1 g DM, respectively. Subsequently, the bottles were flushed with CO₂ passed through a hot copper catalyst for O₂ scavenging and sealed with thick butyl rubber stopper. Anaerobic, reduced and temperature adjusted (+38° C.) artificial saliva buffer solution (Modified from Agriculture Handbook No. 379, (USDA, Washington, D.C., 1975), volume of 37.65 ml), freshly strained rumen fluid (5% inoculum; volume of 2 ml), and test compounds (volume of 0.35 ml) was introduced into the fermentation vessels under the oxygen-free CO₂ flow. Following rumen fluid was collected from a ruminal cannulated cow. Rumen fluid was pumped directly from the rumen into a preheated thermos and immediately closed, and transported to Alimetrics laboratory. The rumen fluid was used for inoculation within 2 hours of harvest. To ensure that both liquid and particle associated ruminal microbes were present in the inoculum both fractions were obtained. Liquid and solid fractions were strained through a metal mesh (grid size 3×3 mm) under anaerobic conditions prior to buffer dilution.

Following inoculation with rumen fluid, buffer and test solution, the vessels were sealed with the butyl rubber septum's. All fermentation vessels were inoculated in random order to prevent the possible block effects. Inoculation time for each fermentation vessel was registered and was taken into account op on each sampling to ensure that duration of the fermentation was the same for each sample. The fermentation was continued for 12 h in a gyratory shaker at +38° C. Fermentation gas produced was measured and relived by collection of the fermentation gas in syringes at time 3, 6, 9, and 12 h. Feed residues were measured after 12 h fermentation.

The trial design was a dosage response, complete randomized design with 27 treatments using 145 fermentation vessels according to table 5. The 24 muramidase solutions were diluted to the concentration needed in enzyme buffer (BSA, Tween 20, acetate-buffer, Calcium, NaOH, adjusted to pH 6). Negative control was supplemented with the same buffer solution that was used for diluting the enzymes to reach the same liquid volume as the supplemented fermentation vessels. The ionophore Monensin (Monensin sodium salt, 90-95% TLC (Sigma Aldrich; Product code: M5273-1G)) was chosen as a positive control because it is a ruminants feed additive that affects volatile fatty acids (VFA) production.

TABLE 5
Treatments, dosage and study design

| | | | | 1 | 2 |
|---|---|---|---|---|---|
| Dosage | N/dosage | SEQ ID | Enzyme class | mg/40 ml | |
| NC | 15 | | | | |
| PC (Monensin) | 5 | | Non-enzyme product | 0.01 | 0.10 |
| A | 5 | SEQ ID NO: 38 | GH24 | 0.20 | |
| B | 5 | SEQ ID NO: 39 | GH24 | 0.20 | |
| C | 5 | SEQ ID NO: 40 | GH24 | 0.20 | |
| D | 5 | SEQ ID NO: 41 | GH24 | 0.20 | |
| E | 5 | SEQ ID NO: 42 | GH24 | 0.20 | |
| F | 5 | SEQ ID NO: 43 | GH24 | 0.20 | |
| G | 5 | SEQ ID NO: 44 | GH24 | 0.20 | |
| H | 5 | SEQ ID NO: 45 | GH24 | 0.20 | |
| I | 5 | SEQ ID NO: 46 | GH24 | 0.20 | |
| J | 5 | SEQ ID NO: 47 | GH25 | 0.20 | |
| K | 5 | SEQ ID NO: 48 | GH25 | 0.20 | |
| L | 5 | SEQ ID NO: 49 | GH25 | 0.20 | |
| M | 5 | SEQ ID NO: 50 | GH25 | 0.20 | |
| N | 5 | SEQ ID NO: 51 | GH25 | 0.20 | |
| O | 5 | SEQ ID NO: 52 | GH25 | 0.20 | |
| S | 5 | SEQ ID NO: 55 | novel MUR polypeptides | 0.20 | |
| | | | having muramidase activity | | |
| T | 5 | SEQ ID NO: 56 | novel MUR polypeptides | 0.20 | |
| | | | having muramidase activity | | |
| U | 5 | SEQ ID NO: 57 | novel MUR polypeptides | 0.20 | |
| | | | having muramidase activity | | |
| V | 5 | SEQ ID NO: 58 | novel MUR polypeptides | 0.20 | |
| | | | having muramidase activity | | |
| W | 5 | SEQ ID NO: 59 | novel MUR polypeptides | 0.20 | |
| | | | having muramidase activity | | |
| Y | 5 | SEQ ID NO: 53 | novel MUR polypeptides | 0.20 | |
| | | | having muramidase activity | | |
| Z | 5 | SEQ ID NO: 54 | novel MUR polypeptides | 0.20 | |
| | | | having muramidase activity | | |

### Sample Collection

Liquid Samples Obtained at 12 h were Analyzed for the Concentration of VFA (Acetate, Propionate, butyrate, and valerate) by GC-FID using a glass column packed with 80/120 carbopack B-DA/4% Carbowax stationary and helium as a carrier gas (Kettunen H., J. Vuorenmaa, T. Rinttilä, H. Grönberg, E. Valkonen and J. Apajalahti (2015): Natural resin acid-enriched composition as a modulator of intestinal microbiota and performance enhancer in broiler chicken. J. App. Anim. Nutr., Vol. 3; e2; page 1 of 9).

Digestibility of dry matter was quantified by determination of dry matter in the original feed matrix and in all fermentation vessels after 12 h of fermentation. Feed residues from the fermentation vessels were obtained by filtration tared sintered glass filters, washed with water and dried at 105° C. for 12 hours. Finally, the glass filters are weighed for the residual feed dry matter.

### Calculations and Statistic

Dry matter digestibility (DMd) was calculated at the ratio between feed residues after end fermentation and the dry matter fed. The sum of VFA were calculated as the sum of acetate, propionate, butyrate and valerate, and the summed carbon in VFA was calculated by assigning the respective number of carbon atoms in acetate, propionate, butyrate, 2, 3, 4 and 5, respectively. Data was calculated as the relative improvement compared to NC for DMd, and the concentration of acetate, propionate, summed VFA, and total carbon in VFA.

One treatment was taken out of the data set, because the amount of enzyme used was not known. Thus, data from this treatment cannot be compared to the rest of the treatments in the current study.

Data was analysis using the mixed procedure of SAS (SAS institute) including the main effect of treatment.

Data presented are least square means and standard error, unless otherwise stated.

### Results:

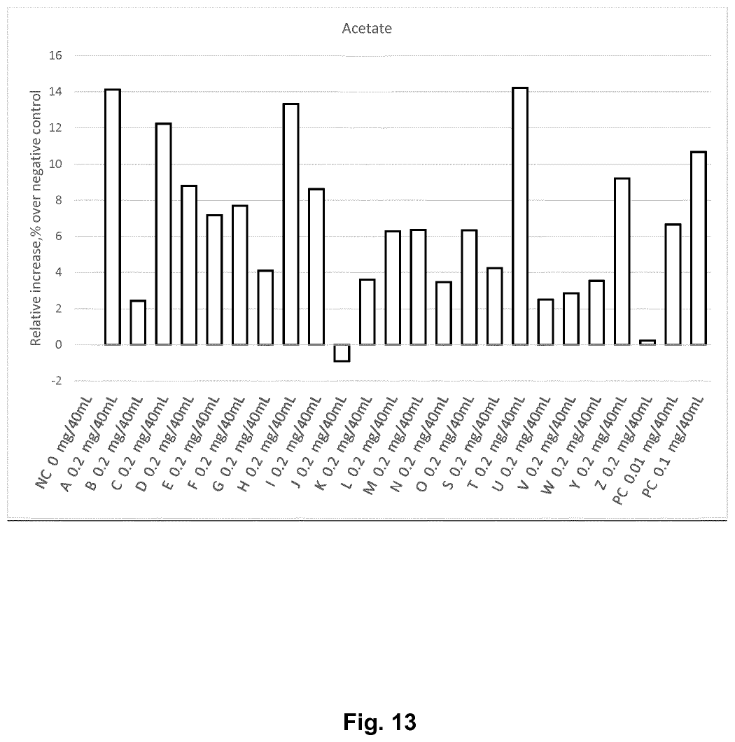

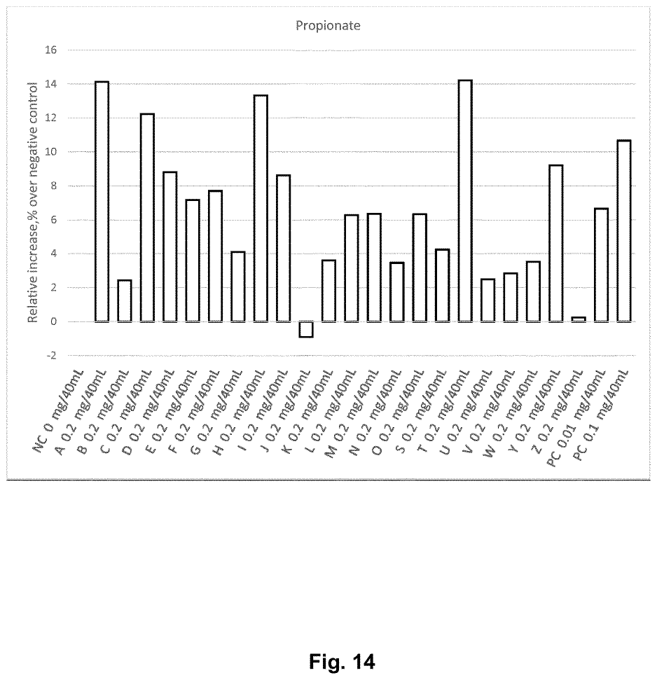

Data shows that ruminal fermentation improved from muramidases supplementation in vitro. This was showed from the increase in total ruminal VFA and total carbon in VFA for 23 out of 24 muramidases treatments table 6. The positive response was divided evenly on the three enzyme glycoside hydrolase (GH) families (GH24, GH25 and novel MUR polypeptides having muramidase activity). Total VFA increased up to 12.2±1.99% and total carbon in VFA increased up to 12.5±2.02% FIGS. 13 and 14, respectively. The increase in total VFA and total carbon in VFA came from an increase in ruminal acetate and propionate Table 6. The acetate fermentation increased with up to 10.7±1.99% and the propionate fermentation increased with up to 14.2±2.21%, FIGS. 13 and 14, respectively.

TABLE 6
Effect of 24 muramidases on dry matter digestibility
(DMd), and volatile fatty acids (VFA) production (mmol/L)
after 12 h of ruminal fermentation in vitro

| | | | | | Total | Carbon in |
|---|---|---|---|---|---|---|
| Item | DMd | Acetate | Propionate | Butyrate | VFA | VFA |
| NC | 0.58 | 48.4 | 39.5 | 7.1 | 96 | 246 |
| PC | 0.51 | 44.2 | 42.8 | 3.7 | 91 | 234 |
| A | 0.56 | 52.9 | 45.1 | 6.7 | 105 | 271 |
| B | 0.60 | 49.1 | 40.4 | 7.2 | 97 | 251 |
| C | 0.58 | 52.0 | 44.3 | 7.2 | 104 | 269 |
| D | 0.57 | 50.1 | 43.0 | 5.9 | 99 | 255 |
| E | 0.57 | 49.9 | 42.3 | 6.2 | 99 | 254 |
| F | 0.58 | 51.6 | 42.5 | 7.2 | 102 | 263 |
| G | 0.56 | 49.7 | 41.1 | 7.3 | 99 | 255 |
| H | 0.59 | 52.6 | 44.7 | 5.3 | 103 | 264 |
| I | 0.56 | 49.3 | 42.9 | 5.4 | 98 | 252 |
| J | 0.56 | 47.6 | 39.1 | 6.9 | 94 | 243 |
| K | 0.57 | 49.5 | 40.9 | 7.2 | 98 | 254 |
| L | 0.56 | 50.7 | 42.0 | 7.3 | 101 | 259 |
| M | 0.60 | 50.7 | 42.0 | 7.3 | 101 | 259 |
| N | 0.59 | 49.3 | 40.8 | 7.2 | 98 | 253 |
| O | 0.59 | 51.1 | 42.0 | 7.3 | 101 | 260 |
| S | 0.58 | 49.4 | 41.2 | 7.1 | 98 | 254 |
| T | 0.57 | 53.6 | 45.1 | 7.8 | 107 | 277 |
| U | 0.60 | 49.1 | 40.5 | 7.1 | 97 | 251 |
| V | 0.59 | 49.2 | 40.6 | 7.1 | 97 | 252 |
| W | 0.59 | 48.9 | 40.9 | 6.7 | 97 | 250 |
| Y | 0.57 | 50.8 | 43.1 | 6.1 | 100 | 258 |
| Z | 0.56 | 48.7 | 39.6 | 6.9 | 96 | 247 |
| SEM | 0.02 | 0.96 | 0.87 | 0.29 | 1.90 | 4.96 |

### Conclusion:

In conclusion ruminal fermentation improved by supplementing muramidases from the three glycoside hydrolase (GH) families GH24, GH25 and novel MUR polypeptides having muramidase activity. The improvement was seen as and increased production of total volatile fatty acids, acetate and propionate.

1. A ruminant feed composition, such as a ruminant feed, ruminant feed supplement or ruminant feed additive comprising one or more muramidases, wherein the muramidase is in an amount sufficient for administration at a level of 1 to 200 mg enzyme protein per kg ruminant feed.

2. The ruminant feed composition of claim 1, wherein the ruminant is selected from the group consisting of: cattle, dairy cattle and beef cattle.

3. The ruminant feed composition of claim 1, wherein the muramidase comprises one or more domains from a glycoside hydrolase (GH) family selected from the list consisting of GH24, GH25 and novel MUR polypeptides having muramidase activity.

4. A method of improving the Energy Corrected Milk (ECM) production of a ruminant comprising administering to the ruminant a ruminant feed composition according to claim 1.

5. A method for increasing dry matter digestibility (DMd) of a ruminant feed, ruminant feed supplement or ruminant feed additive comprising the steps of: a) providing at least one muramidase; b) providing a ruminant feed, ruminant feed supplement or ruminant feed additive suitable for a ruminant animal; c) applying the muramidase to the ruminant feed, ruminant feed supplement or ruminant feed additive to form a ruminant feed composition; and d) feeding the ruminant feed composition to the ruminant animal, whereby an increase in dry matter digestibility is effected.

6. The method of claim 5, wherein the production of volatile fatty acids (VFA) in the rumen is increased compared to the production of VFA in the rumen of a ruminant not fed with a muramidase.

7. The method of claim 5, wherein the production of propionate in the rumen is increased compared to the production of propionate in the rumen of a ruminant not fed with a muramidase.

8. The method of claim 5, wherein the production of acetate in the rumen is increased compared to the production of acetate in the rumen of a ruminant not fed with a muramidase.

9. The method of claim 5, wherein the muramidase is dosed at a level from 1 to 200 mg enzyme protein per kg ruminant feed dry matter.

10. The method of any ono of claims 5 to 9 claim 5, wherein the Energy Corrected Milk (ECM) production of ruminants upon administration is improved by at least 1.0%, preferably at least 1.5% more preferably at least 2.0% compared to control.

11. The method of claim 5, wherein the muramidase is of microbial origin.

12. The method of claim 5, wherein the muramidase comprises one or more domains from a glycoside hydrolase (GH) family selected from the list consisting of GH24, GH25 and novel MUR polypeptides having muramidase activity.

13. The method of claim 10, wherein the control is a ruminant feed composition which does not comprise muramidase.

14. The method of claim 13, wherein the control is a ruminant feed composition which does not comprise GH24 muramidase, GH25 muramidase or novel MUR polypeptides having muramidase activity.

15. The method of claim 10, wherein the control is monensin.
